SEQUENCE LISTING (91 sequences)

SEQ ID NO: 1 - RNA, 1527 nt, Streptomyces sp.
  source 1..1527: mol_type = genomic RNA
acggagagtttgatcctggctcaggacgaacgctggcggcgtgcttaacacatgcaagtcgaacgatgaacttccttcgggaggggattagtggcgaacgggtgagtaacacgtgggcaatctgccctgcactctgggacaagccctggaaacggggtctaataccggatacgacacgggatcgcatgatcttccgtgtggaaagctccggcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggaatcgtcgaaggtgggactggcgattgggacgaagtcgtaacaaggtagccgtaccggaaggtgcggctggatcacctccttt

SEQ ID NO: 2 - DNA, 681 bp, Streptomyces sp.
  source 1..681: mol_type = genomic DNA
atgagggagcagaagatcatgtcgcggttgtctgcatccaattcagttatgtatgaactcgcgtctcaaaatccgaacatgtcggacctggatcggctcacgaacctctactgggcgctgtgttccgtacttcgcgccgaagtgcccggcgaagtggtggagctggggtgtaacgcgggactcaccagcgtgatgctggaaatggtgttggaggccgagtccccggaccgtacgcttcatctgtacgattccttcgacggcctgcccgctccttcgcatgctgatcaatatttgcgggagggggactgcaaagcgaccgaggcagacgtctgcgccgcatttgagcgctggggcctgcgccggccggagatccacgcggggtggttcgatacgacgctgccgcaacagctccccgaccgcgtggcattcgcctacctcgacggggacttctacgaatccatcctcacgagcctgacacacgtctggccgcggctgtccgtcggcggctccgtgctggtggacgactactgcgacaggctggcaagtccacgcgcttgggacgggctgcccggcgtgaagaaggcatgcgacgcgttcttcgcggacgttcccgtgcgttcacgcgtcctcgtcggcgcaggggacctggctttcgccgagttccgcaagaccgaagcctga

SEQ ID NO: 3 - DNA, 669 bp, Streptomyces sp.
  source 1..669: mol_type = genomic DNA
gtgctgcacgattcgttgaagatcggaagcgtcgaaggcctgctgtctccggatgaggtcacccggctgacggcctgtgtcaccgacgggctgggcgccgaccgccgtttcacggtgtccgaacgccggcggtcggtgcactcgctggaaggtgagagcctggcgcgcgtcaaagaagtctatgaaccgcacggccgcgtggaatacgacgagttgccggacgagatcaccggtgtcctggagggcgcggtggcccgtcgtctcggtgaactgcgaagaatctttccgaccgtccgccgttccatggactggctctacgtggaatacggagccggtcagtacgtcaccccgcatgtcgactacccgttcaatgagatcgcaccggaccggcccaaggtcgccgccataggaatcgtcctgcacaaggccgaagcaggtggcgaattcttcgtggagaccggcggcggcgacgccctctggaacgatgaaggtgaggtcaaggaaggcgccaacgaccacagcccgtggttcaacgccatgcgccgtacccggtggcacgtcaccagccgcgccggggacgcggtgtgctggggtactcaactcgtgcacggtacggaaccggtggccgcgggtcggtccggcaagttcatcggttttctcggctcctga

SEQ ID NO: 4 - DNA, 891 bp, Streptomyces sp.
  source 1..891: mol_type = genomic DNA
gtgaagacggacttcccttaccacccctcacaggaccgccgcggcgaccggctgacgccgcccgtcgccgaacgcaacgcccgccgttcacgaggtgtggccggccgcatcgtatgggcgcactcgctgtggatcgacccggagctggagcggctgtacggcggcgaccggcatgctcccgacgtcaaccgcgaacgggcggtgcggctgtcggacggccgcctggacccgctcgccatgcgccccctggattctctcgtctcggtcaccggccccttcctgagccggcacaccagcaccgtcggcgtcgatccggccgacgaagccttcgacctggtccgccgcacgcacagcggttacctgtgcgaactggccatcggggtcatccggccgggctccccacagcgaggggtgacgccgtcggggccggaccaccgcgaacgctcgcccttcaccgccgtcgtcccccgcgaccacgatgcggtgagccgggccgccgggctcctcgaatccagcgcgaacgagctgctcagccagtggtacgaagccttcacccacgcgcgcggcaccacgcacggcatcctgctgccgccactgcgctgggcacagggcgtgttcgcgctctccatacgctacgaatactgccgccgggtacgggacgagccgggtttcacggccggggagcggcagcacgccgggctgctgctcgacgaggtcaccgaagccctcccgcaacccggcgccttcagggacagcctgcccggtggcgtccagggatcggtggtcaacgaactgctgtcctgctcctaccccttgtgggaccgggtccggttctgcctggactcccccttcgacggactccggtccctcgcggaacattcgacggcctga

SEQ ID NO: 5 - DNA, 720 bp, Streptomyces sp.
  source 1..720: mol_type = genomic DNA
atgagtcacgccgcatttccggcggtggaatccgccacgcacaacgcggaaggcgagcacggccatcaccgcgccaggtttccctatgtcgcgctggaggggctcaacggggcgggaaaatccactctcaggaacagcctggaggaatccttctgggccctgggcatgaatgtgtacaccataggccagcattcctggcagtcaccgggggcggcccgcgtcatcctggacatcaaggaacgccgccacacgcacccgccggagcgcatccaactcgcctacctcgacgaccggcgagcccagggtgacaacatcctctcccgagtactgcacgaagtgccggtcatcggggaccgctccctggtgagcgacatcgtgtatctcgaagccctggacggcatccccgccgaggacaccttcgcgctctaccgccggcatgcggtgatcctgcctgatctgatcgtccacgtcgacgtgtccgcagccaccgccctggcgaacctggggaaacgagaacgccggcgcaagccgcacgagaatcctgaacagatgcggtcgctggccgctacctatgaatccgtgctgtgggcgcagcggttgccgggtctgccacccgtgctgcgatatcagcccgaagtggccgcggacgggacgtcgtacacgaacccggccgcctccatcggcgaccgcatcctcgcttctctgggcctggcctcctga

SEQ ID NO: 6 - DNA, 585 bp, Streptomyces sp.
  source 1..585: mol_type = genomic DNA
atgagcgcaccagggcgggcgatcgtactcgccggaacatcgggagccggcaagagctcaacggccgaagaactccagaagacacttcccgatgccttcctcgtcctgggcctggacaccttcttcggcatggtgccgtaccggtggaccagcgcgggaacggccagtgccgagggtttctcctacatccgggacaccgatccgcacgacggtgccgcgcgtctgcggatcggctacggcccggtcggcgcgcatctcttggccggcatgcgccgggcgacggaggccctgctgcgggagggaaacaacgtcatcgtcgacgagatgccgatcgactccggagtcgttccggcctggaagcgggacctgctggggcacagcgtgttgtgggtccacctcacggcgccgttgccggtgctggaagcaagagaagccgaacgcttccccgagaagttccgtggactcagcagagggcacttcgacgtgtgtgtcccggcggacttcgacatcgtcgtcaactccgccgagctcgatccgagggaacgcgccgaccgtatcgcggcagcgcttccgccccgtcgttga

SEQ ID NO: 7 - DNA, 1227 bp, Streptomyces sp.
  source 1..1227: mol_type = genomic DNA
atgtccgacgcccggtccgcgacaccggccgtgccgttgcgacgcaaccgtgactacaacctgtggtggtcgggcgcggcagcttcccagaccggtagtttcatggtgaccgtggccgtccccatgctcattctgaccaacagcgggtcggccggtcaggccgggctggtcaccggtgcgtcggccgtcgccaaattcgtcgttgcctttccggcgggtgcggtggccgatcgctgttcccgccgccgcctgctcctcctgtcctctctcatgcaattggccgccgtggcctcgatcctgatcgcggtggcctcgcagcacgtccggctcgtccatctggcagcggccgcgcttgtcgagggggcgggttatgcccttcattcggcaacggagctaccgctgttgcgaaaagtcgtgcccgagccgcagcgccgggccgcgctcggtcgcgagcaagggcgaaaggcagcggcacagctgaccggtcctccgctgggaggcacgctcttctcatgggtcccgtgggcgccgttcctcgcggcctcggtctccttcctcggggtgaccacggccgcgctgggaatacgggccccgctgggcccgcagcgccggcgtaaggaagccgcgccccccttctttcccgaaatgctcgaaggagtgcggtatatccgccgcagtgccttcatgagctacatgctcctgtggttcgcgctcgtcaacggcgcattcgcgggcctgagctttttgctcgtcgtgctctgccacgaccgcggcgcgtcagcctccgccgtgggcacggcccagggcgtcggctcggccggggcggttctcggagccctcgcctcggcgtggctgaccgcacgcctgacgcaccggcagcttctgaagctcgcttcgtgggtgctggtcatcggctccgccgccatggccgtgcccaccctttccccgctccccaccggcctggcatacggcgtctccctgttcctgacccccgcggtgaacgtctcgttcacgcatcacatggtcgggaccgttcccgacgctttgaccggacgcgtgtcgatgaccatgatgacggccgcacgcagcctgaactggctgttcgtcatagccgtggggttggcggccgagaggtgggacccgctcgtccccctcttcaccctgtccagcctgttcctggccctggcctgcgccaaccacctccggcgtgtgcgcacacaacggtga

SEQ ID NO: 8 - DNA, 129 bp, Streptomyces sp.
  source 1..129: mol_type = genomic DNA
atggccgtccgccccgtcatcaaggcccgtaacacccatctgatctgcgaaaacgcggccagaggcggggtgcgtgacgcgcgcggtgtcaccgtcgtcccggccttgccgctgcgggcgcaacggtga

SEQ ID NO: 9 - DNA, 3378 bp, Streptomyces sp.
  source 1..3378: mol_type = genomic DNA
ttgaccacatcgttatggagcagacgccgcttactcagcacactcgccggagcggccgcggtccccgccctgccgtccatcctcacgctgaacgccggtgtcgcccatgctgccgggccggccgacggcaacgggccgggggacgtcctgcccacgaccgagcgcggtcaggcggtgtgggcgtacaagaccggaggccgggccgtccgtgaggcggccgccgccgcccttgtcggcagtccggccgccgtgaccgccttcctcacccgcgaactgcccgcggcgagagcggaagacaaccgcttcgcgatcatcatgggtctctcccagggcggcaaggccgtcaacgaaacggccggcgcggcactgaccgcgggagacgaagcggtcgccgcctacctgcgggacggatacaagaagcccgtcgaagaggacctgcgggtggcggtgttcaccgtcatgaacaacagcggtgtcgccgtgcggcgggaggccgacaaggcactgcgggagaacacccggtcggccttccagaagttcctgggcgagacccagtacaccgcgcagcaggaagacgagcgggtggaggtcttctccatcctcaacaacgcctcgcccgaggtgaagaagtacgcgcagcgcgccctgagcgagggaacgcccgaggcgatacgccagttcctcagctcgggccagcacatcgcccgtgcgcgcgacgaagagcgcgcgacgatcgaccagttggtcgagatcgtgcagcgtgagagcaggcaggcgaagctgcagacggagaacgcggtcaccgcctccgaacgcgcgaagagcgccgccgagaaggcgaaggcggccgcgctgctggcggcggagcacgccaaggcggccgagcaggacgtacgcaagtcggccgatgcggcgaccagcgcggcgagagccgccagtggagcggccgatgccgccggcgccgccatcgcggcctcgcgcaccgcgcagtccgcctcccaccgcgcggctcaggccgcccaggccgccgctgccgccgccgcgatggcgggacacgccgcttcccgcgcctacaacgccgccatcgccgcttccaaggatgcctccaaggcgtccgccgcccgggtggccgcccagggcgcacggaacgccgccgcacaggcgcgtaccgcggccaaggcggccgaccaggcgaccatcgcctcccagcaggccgctgtcgccggcaccgccgccgcgggcgcggcccggagcgccgcctcggccgcgaacgccgccgccgagtccgccgccgccgcgggcgccgcccagtccgaggcggccaaggcgaggcaggcggccgccgaggccgatgccgccgccgcacgggccaccagggccgcggctacggcccggtccttggcgaaccaggccgcggaggccgccggcgtggcacgcgacgccgccaacagcgccgccgaccatgcggagaaggccgccgccgcggccgaggaggccgcgaagaacgcgggcaaggcagtggagtacgccaagcgctccgccgcctacgccgaagcggcgatggaagcggccaccaaggccgacaacgcggtcaagaaggctctgcaggtggagaaggcggcgcgcgaggcggaagccgcccggatcgccgcggacaccgaactcggggtgctggaggcccgcctccgggcgaaggcggagaccgaggacgccaagcgcgccgagaggcagcgggcgcaggcggaccgggccacgcagcgggtcaaggacctgatcgccgctgccgaggcggcgctgcgggccggtgacaccgccaaggccgtggcgtccggccgccaggcagcggtcgggctgctggagtcttccggcacctggaccagggaagccgcggagttcgcgctcgccggcaccgaccaggacaccgtcaactggatcgacaccgaccgccttctcgcccagcagcaggacgaccgggagaccgtcctgacgatcgccacgtcgtccacggccgacgtggccgaggccgcccacaaggtgctggccggcgattccccggaggcggtacgcgacttcctcgaccgcggcgtcctcgaagccgcggccacggacaaccgcgtcgccttgttcagggtcctcgacgagaacccgggcagagccgtcagggagaaggccgaagcggcactgaaggacgggagtgcccaggccctgcaccgtttcctcaccgaggaactgacggaagcggtgaaggaggacgacagcgtcgaggtgttccggctcctcggctccggcggcccgtacatgcagtcggcggcgaagatcgtcctggagggctccgcccgtatgcggcgtggcttcgtcagccacgaccagtacaacatcgcgcggctcgatcacgaccgggcgacccatgtcgcagccatccagtcgtccatcgccctcgccgccaaggtcgccgcggaagcgctggagagcgccgcccgggcctccaaagcggccgcggaggcccgcgacgcggccgcggacgcgaccgactgggcgaacaaggcggacgcctacgcccagagggccaccgattcggccacggaggcgaggcgcaacgcggacgccgccgacaagtcggccgccgccgcggcccagtcggcgaaggaggcgagcaacgccgcggtcacggcacgtggtgcggcgcgggcagccaacttctcgatgcggcaggcgtcggcgtccgcccagcaggcggtggcgtccgcgtggcaggcacaggcttccgcctcccgggcccaggcctccgcgacccaggccggcaaggccgccaaggccgctgccgccgccgccagcgaggcacgccggatcgtcttcgtgaagcggaaggccgaggccgaggccgcggcgaagagggcggccgagaaggcgaaagagcaccagtcgaacgggaccaacccctcccagacctccgacaacgacaagccctggtacgtggagaacggggtctggcccgaggacgtgaagagcgccaaggactgggctcaggtcaccgggcactggagcacgatcgccggcggggccgcggtggcgctcggcgtcggtggcttcttcttcgctccccttgggggagccgccctggcagtcggtctggtttcctggggactgcaaggagcgagcgccttgctgaactacatcgggaatgggccggacagctccgaattccattcggcactcggtatgttcgcggtcggcggtatccttctgggaacggggcagttgctcagcaagttcggcaaaatgccggcattcgggcccgcggtcgaacaggtcggcgagaagatagccaacgtcgcccatgacatcacgaccacggtggccggctggctcacctggtag

SEQ ID NO: 10 - DNA, 378 bp, Streptomyces sp.
  source 1..378: mol_type = genomic DNA
atgctgacatccacactgtcagagacccaatggtgggtggccgccgtcctgtccggattcgctctcgcggtggcggcacagtccatggccaccaccgcgcgggacaaactcatcgcggcatcgctcccgctggccggagcgatcgcgatcggtgtgagttcggcggcgcgcggttacacggacacactgccgctgctgctctacaccaccaccatgatcgtcttggtgattctgcgactgctcttcgccaagtacatcaaccgacagctcgccctgaagcgctccgggaaaccgatggagaacatgtcgggcaagcaaaccgccatctttctcctcgccttcatggccacgctcgtcctcgtgtccttcgtcatctga

SEQ ID NO: 11 - DNA, 504 bp, Streptomyces sp.
  source 1..504: mol_type = genomic DNA
atgagaatcacacggcgcctcatcgtcacgggcacagtcctcgcagcgagtgccgcaaccgccgtcaccgtggcccaggcgacgggtaatccggcaccggccacggtcaccgtcgccggtgctgagcccggctccgccgtcgaggacttcagttacccgaatgccgacaagatcaaaaaggaactcggcatcgacctcaagcgcggcgacgggcacatcatgctcgtcgactgcaacaatgacggccgcctgatgaccatctggtcgcgcaccatcgggcaggtctgcttccgcgtcaccggcgacaccggatatctgagcctgtcgattcccaaggtgtacggagtgaaggcagacgccgatcacaaggccaacctgacgctgaccgctgacggcagcaagcagaacatcgccttgggcaagaatgagtcggggtcggccggagagaccgccgacccggagcgtcgtccgcacgacctcgtcgaaatatccacctccaagtag

SEQ ID NO: 12 - DNA, 150 bp, Streptomyces sp.
  source 1..150: mol_type = genomic DNA
atggacgtccagatgagcaccgaggccgacagcgacctcggtgaactagaggtacttgagatggatgaactgcaagtcagcggttcgtccacggcctacaactacatcgccaagaacgcggaccgctcttcgggtataccggcgggctga

SEQ ID NO: 13 - DNA, 840 bp, Streptomyces sp.
  source 1..840: mol_type = genomic DNA
atgatctgctgcccgccgaggcagatggatgtgcccgggcagagccgccggatccaccgggcgatcagcagagccggcgccaactggctgaagaaggcaaccgatatgccgaccgcccgcggcttggacgcgtcaagcgcctccgccagcgctgcgcgcaggttccgggtcgcgaagtcgagagagccggcctgcggctcggacaaccggagcacggcctggtcaagtgcgtcgaagtcgtcccatacctgcgacagcgccgattcgcggagtatcagctgggtggcctgccgggccagacgcaccgcacgcacgggaccgaggcggggcgccgggttcttcgtcaggaaggtgaactcctacctcaatgacaccgttgtcctctccatggtgtcctgtgtgggctgttactggggacgctgcatcttctgttcctacggaaaccgctctctcgaacgcggggcctatcagcaggcttccccggggcaactcgccgacacggtgtgccggatcgtgaaaagtaccgggatcgacttcgtcaccgttgtcgatgagaatacgaacctgctactgctcgtccgggccatgcggttggtgcgtgagcgtggccttcgggtccgtttcaatacccgcaaccgactggaaccgatcctgctcgactctgccttctgccaggaactggccgagctgggctgcgagggaatggctgtcggatacgagggggtcacccagcgactgctgttcaccgtgctcgatctgcggatcgccgatttcgggcggcaggtgggtgttacggactggccggcccggtacgccgtgctccggagacaggaggactga

SEQ ID NO: 14 - DNA, 774 bp, Streptomyces sp.
  source 1..774: mol_type = genomic DNA
atggacgacacgaccttgatatcccgattcctgcgcgacggcttcgtgaagctggagggtgccgtcgcgccgcgcgtggccgcggactgcgcgcggctgctgtggcgggagacgggctgcgacccggacgacccgacgacgtggacgcagcccgtgcactgggtgcccggcctggcgcaggggccgttcgccgccgcccccaactccccggccctgcaccacgcgtacgacctgctcgtcggcgcgggacgctgggagccgcgctactcgctgggcaccttcccgctgcgcttcccgcacgagcaggagccggacgacgcgggctggcacatcgaggggagctatctgccggagggcgagagctggtacttcaccaatctgcgctcccggggccgggcgctgctgatgctgttcctgttcagcgaggtcggtgaccaggacgccccgacccgcatccgggtcggctcacacctcgacgtgccgaaggtactggaggggtacggggaggacggtgccagcgggctgaccctggcgcccgatctggtggcggcgtccgaccaccggccgctcaccctggccaccgggtccccgggcgatgtcttcctgtgccacccgttcctggtgcacgcggcgcagccgcaccacgggaggcggccgcgtttcatggcccagccaccgctgatgccggccgcgccgtacgaactggagcgggccgacggcgcgtactcgcccgtggagatcgcgatccgccggggcttggggcgggacaggtga

SEQ ID NO: 15 - DNA, 180 bp, Streptomyces sp.
  source 1..180: mol_type = genomic DNA
atggcacgtcggcaaccccacgcacccgagcgactgctcgtcgccgcccatcctgaactgcgcgaacgcgaggcactgaaggaactcggcctcatcgcctcgatgaccgacgcgctcaagcgccgcggcgttcccgacctgaccgcacgcgtggccgccgagctgggcgcgctcgcctga

SEQ ID NO: 16 - DNA, 945 bp, Streptomyces sp.
  source 1..945: mol_type = genomic DNA
atgagccgtgctgtggtctacgagtcgttcggtggtcccgaggtgctggagctgcgggaggttccggagccccatgccggcccgggcgaggtccgcgtccgtgtcacggccgccggcctgaatcccatggactggggcatcgcctcacagcccgaggccgccgcccggttcggcatcgccgtgccggccggcttcgggttcgacttcgccggggccgtcgacgaggtcggcgccggtgccgaggggttcgccgtgggggaccgtgtccacggcggcgcgctgggaagggccgttgccgatttcgtggtggtcaagacgcccaccgggacgaccgacccgctctggcacacgccggagggcatcagcgacgaggtggcggccacactgccggtggccgggatgaccgccgctgccgcgttggccgcgatcgacctgcgttccggtgacaccgtcctggtcggcggcgccgcgggcggcgtgggtgtcttcgtcgtacagctcgctcagctcgccggggccagggtgatcgggaccgccggccctggcaccttcccgttcctccgccggttcggtgtcgagccggtgacctacggccccggactggcggaccgggtacgcaccctggctccggagggcgtgaccgcggcgaccgacctgttcggcatcgaaacggccgagaccgcgctcgcgctcggcgtgccggccgagcggatctccacgatcgccgcgggccccaaccctcccggcggcgcccgcgcgaccggcggtttcgacgcgagcccggacgccatggagcggatcggcgacgcgatcctcgccggtgcgatcacggtaccgatcgccgccgccttcccggtcgagcagatccgcgaagccgtgacgttgcaggccggacggcacgtccacggcaaagtcgtgatcacgctgcggccggggccgcactga

SEQ ID NO: 17 - DNA, 201 bp, Streptomyces sp.
  source 1..201: mol_type = genomic DNA
gtgcagacgctcgctcccctgctggcagagggcagcggagtggtgctcacggcctcggccgtcaacgtactgggcctgccgatgctcagcgcgtacgcggccggcaaggcggcactgcgctcgatgacccgcagtctggcccgggaactgctcccgcgaaaccgtggacggcggtggcactcagctctgacacccgtctga

SEQ ID NO: 18 - DNA, 867 bp, Streptomyces sp.
  source 1..867: mol_type = genomic DNA
atgcatactgcagcggtgagcgacgtgccggtggggaacagaacggagctgggtgacttcctgcgcagccgacggggccgcgtcgcaccgcaggaggccggtctgccgccgtcggcgggccgccggcgtacggccgggctgcggcgtgaggaggtcgcgatcctggccggtgtcagcatcgagtactaccgacgcctggaacaaggcaaacagcgacggccgagtcccgcggtactggaggcgatcgggcaggtgctcaagctgaccgacgacgagcgccgccacctcgcggagctgagccggggccgcccgccgtcaccggtcaccggcgcgcggacgggcagggcgtcgcgggcggtacgggaggaggtccgccggatgctggagatcgcgcacccgtaccccgcctgtgtgctgagccggtccagtgacctgctgaccgccaaccagggtgcgctgcgcctctttcccggcatcgaggactggcccgagccggagcgcaacaccgcgcgctacgccttcgtccacccccgcgcccgggaactgtacgccgactgggacgacgtggcccgcggcgtcgtggcccatctgcgcgccgccgtggtggtgcaccggggcgcaccggacatccgcgaagtcgtggacgaactctccgcccgcagcaccgcattcgcttctctctggcagcgtcacgacgtccgggcgcgcaccaacgggcgcaaggtgtaccaccacccggacgcggggcggctgacgctgacgtacgaggcgtacgacgtggcccgcagcgaaggccagcggctgatcgtctaccaggcgccgccgggaacaccggaccacgaggcgctgctccgcctgtcggcctga

SEQ ID NO: 19 - DNA, 1485 bp, Streptomyces sp.
  source 1..1485: mol_type = genomic DNA
atgggcggcgcggaggcccacgaagaagggacggtgggtccggccgtgtccactcaagccgaaccgacccgccggggacgccaactcgtcctggcggtctgctgcacgagcgtcttcctcagcagcctcaacaacacggtgctcaacgtggcactgccggcgatgcagcgtgatctgcgcagcccgctctccggactccagtggaccaccgacgcctacctgatcgtgctggccgcgctgctgatgctcgccgggtccgccggggaccggctcggccgccgccgggtcttccggaccggcctggccctgttcaccctcggctcgctcctgtgcgcattcgcccccgggctgggctggctcatcgccttccgtacggtccaggcggtcggggcctcgatgctcaacccggtggccatgtccatcatcaccaacaccttcaccgcaccacgtgaacgcgcgcgggcgatcggtgtctggggcgctgtgatgggtgtggcgatggcggtgggcccgttggtgggcggggtgctcgtccagggtctgggctggcggtcgatcttctggctcaacctgccggtcggggcggccgcgttcgtctgcgccgcgcgcttcgtaccggaatcccgcgcaccccggccgcgccgggtggacccggtcggacaggccctggtcgtcgtgctgctgggctgcctgacgtacgcgatcatcgagggccccggcaccggctggaccgccccgcgcaccctcgcctgcgccctggccgcaccggccgcgctcctcgcactcgtcctccacgagcggcggcgcaccgaaccactggtggacctgcgggtctttcgcggcgccgggttcagcggagccagtgccatggcggtcctctcgttcttcgtactcggcggcttcctcttcctcaacacgctgtacctgcagaacgtacgtggcctgtcggccctggcggcgggcctgtgcctgctgcccatggcggtcatgaacatcgtcacctcgccgctctccggacggctgaccgccacccgcggcccccggctgccgctgctcctggccggtgccgccaccgccgcgggcggtgtcctgctcgccaccggcggcgcgctcacgacggacgcgctgctcttcaccgcgtacgtcctgatcggcgtcggggccggactggccaacacgccggtcaccgacgcggccatgtccggcatgccgcgggaacaggcaggcgtggccgcggcggtcgtcgccaccagccgccagttcgggcaggcgctgggggtcgcggtgctcggcgcggtactcaccgccggtctgcaccgctccggtacgccgttcacccgggcggtgctcccggggtactggatcgtcaccgcctgcggcctggccatcgtcgtcctcgccctggccaccacccgccgtgtcgtgcctgccgcctcaccggccgggcgacgtcgccggcaggcggccggcgcgcgcctgggctag

SEQ ID NO: 20 - DNA, 615 bp, Streptomyces sp.
  source 1..615: mol_type = genomic DNA
atgcccaagacggtggaccgggacgaacagcgccggcagatcggcgccgccctgctgtccctggcgtccgaacgcggcctggacgaggtgagcgtccgtacggtggccacgtactgcggccgctcccccggagcggtgcagaagtacttccgttccaaggacgagatgctcgccttcgccgccgagatggcgggcgagcgcatcgaggcacggatggccgccgtcgaccccgggctgccgccgcgcgcggccctgcgggccctggtactgaccaccctgcccctggacgccgaacgccgggccgaggcagcggcccaactggccttcgccgtccgcgccgcacaccacgaacccctcgccgaagtacgccgccgggtcgaccaggacatccgggaggcactcgcgcactggctcacctccgccgggtacggccacggccccgacccggccacggactgcctggcgatcgccgacgcggtgatcgcactctccgacggactggcactgcgcctgctctacacacctcaggcccacgacgacctggtacgcgccctggaccgcgggctggaggcgctggtcccggcacaggcccccggcgagccggacgcacgctag

SEQ ID NO: 21 - DNA, 279 bp, Streptomyces sp.
  source 1..279: mol_type = genomic DNA
atggacgaccggctcgatcctgtcacgcgcgcatcggaccaggaccgggagaaggcgctcgcccgggaacgcgaccgcaaggacctgcgggagtggctggccgagtggcgctgctggctcggcggcggggtgatcttgtcggcggtctggggcgtgcggtgcctgcggaagggcgaactgggcttctactggcccgtgacgccgttggggatctgggccgcggtgctggtcgccatcgcggtgtggccgcggagcgggggtgggggcggggaggggtga

SEQ ID NO: 22 - DNA, 23097 bp, Streptomyces sp.
  source 1..23097: mol_type = genomic DNA
atgggaagggtagtgcccggtactccgcaccacagccggcccctgtcgtcggcgcagcgtggcatctggttcgcgcagcagctcgacccggacagccccagctatcacaccgccgaatacgtggagatcgaaggccccctcgatctgccggcgttcgagaccgcggtgcgccgggcggtgacggagaccgactcgctcaacgtccgcttcaccgacgacggtcaggggccctgccagacgatccagcgcgggggcacctggtcgtaccgcgtgttcgacgtcggcgcggagccggacccgagggcggcggccgaggcctggatgcgggcggagacggaccggccgggaggcctggaggcgggccggacggtcactctggcgctgctgcgggccggggccgaccgtgccttcttctacctgcgtgcccatcacctcgtcaccgacggctacatgtgcgcgctgttcacacaccgggtggcggagctctacacggccctggtcaccggggcgccgcagcgcgaggacagcagcggttcgctggacctgctgctggccgacgacgccgcgtaccgggagtcggagcgcttcgcgcaggaccgggcccactggctggaccggctggcgggccgccccgagccggtgaacctcgcgggccggacggcccgggtctcgcggagcttcctgcggtacggcacccgcgtaccggacgccaccgccgacggcctgcgcgcgctggcgcgccgggcgggcgtgggcctgccggtccccgtgatcgccgccacggcgctctacctcggccggatgaccggcgccgacgaggtggccctcggcatcccggtgaccgcgcgcagcggcggcggacggcacatccgcgggccgatgtccaacgagctgccgctgtgggtggaggttcggcccggcatgagcggccgggacttattacgccaagtgtcggccggagcgcggcagttgctcaggcatcagcggtaccggtacgaggacatcgtccgcgatgtgcggacggtcggcggcgagcgccgcctcttcggcccgaccgtcaacatcatgtccttcgccaccgaactgaccttcggcgaggcccgggcgaccgcccacaacctgtccgtcgggcccgtcaccgacctctccttccgcctcgacgaccgcctgggcggcggcgcgctgcacctcgacgtcgacgccaaccccgccctgtacgacgcggcggagaccgccgcgcatcagcggcgcttcctgcgcgtactggagagcttcgccgcggccgacccggaacgtcccgtcggccgtatcgacttcctgctgcccggtgagcgcgccgaggtgctgcccgtacggccggcggcgacggacgctccggagcccgacgagcctggcccgacgctgccgacgctgttcgaagcgcaggccgcgcgcacccccgacgcggtcgccgtcgtcactgaagacggcgagactgaagacggcgagctgacctacgccggactgaaccaccgggccaaccgcctggcgcgcctgctgctgaagcggggcgcgggcccggagcggttggtggccgtggcgctgccgcgctccgccgacctggtggtggcgctgctcgcggtggtgaagacgggcgccgcgtacctgccggtcgaccccgcccacccggcggagcggatcgcactcatgctccgggacgccgacccggtgtgcctggtcaccacgggcgaaccggccgccggggccccgtacggagcggacgtcgtacggctggacgacgacgccaccgtacgcgccctcgccgacgggcccggagacgacctgaccgacgccgaccgcgccggacagctcgaccccgcgcacccggcgtacgtcatctacacctccggatcgaccggcacccccaagggcgtcgcggtcccgcaccgcaacgtcgtgcggctgttccggcagaccgaggcatggttcggcttcggccccgacgacgtgtggacgctgttccactcctacgccttcgacttctccgtgtgggaactgtggggggcgctcctgcacggcggccggctggtcgtcgtcccgcaggacgtgagccgttcccccgagcgcttcctcgacctgctcgtacgccaccgggtgacggtgctcaaccagacgccgtccgccttctacccgctgatacgggccgaccacgaccacccggagctgtcggcgcggctggcgctgcgtaccgtcgtcttcggcggtgaggcgctggatcccggcaggctgcgcgcgtggtacgagcgccatccggccgacgcgccgcgcctggtgaacatgtacggcatcaccgagaccaccgtgcacgtcacccacgccccgctggacgagcgggacgccgccgccgacccgccccgcagcgtcatcggcgagccgatacccgacctgcgggcctacgtgctcgacaacgccctgcggccggtgccgccgggcgtcaccggcgagctgtacgtggcgggccccggcctcgcccgcggctacctcgggcggccgggcctgagtgcgcagcgtttcgtggccgacccgttcggccgccccggggagcggatgtaccgcaccggggaccgggcgcgccggcacgcggacggcaggctcgaccacgccggacgggcggacgaccaggtcaagctgcgcgggttccgtatcgagccgggagaggtcgaggccgtcctggcccggcacggggcggtggcgcaggccgtggtgctgctgcgcgaggaccggcccggcgaccggcggctggtggcctacgcggttcccgccggcgggccccgtccggagtccgccgcactgcgcgagttcgccgggcggcacctgccggggcacatggtcccggcggccgtcgtcgtcctggaccggctgcccctgacggccaacggcaagctggaccgggccgccctgccgtccccggagtacaccccgggtgcggcgcaccgcccggcgcgcgacgcggccgagcgggtgctgtgcgacctgttcgccgaaatcctcggcgtgcggcgggtgggcgtcgacgacgggttcttcgacctcggcggggactccctgctggcgacccggctgctgagccggatacggaccgcgctcggcgccgaactgccgatccgcgtcctgttcgacacgccgaccgtggccggactggccacggccgtacgggagtcgggcggggaagcccggccggtggtgcggcgcacggagcgccccgaggcgctgccgctgtcgttcgcccagcggcggctgtggttcctcaaccggctggagggccgcgaggccggagccgactacaacatgccgttcgtcctgcgactggaaggtgcactcgaccgggcggcgctggaagcggcgctcaccgatgtcgtcacgcggcacgagagtctgcgtacggtcttccccgaggtcgacggcgtgccacgacaggtggtgcgtgaccccggagccgccaccgtcaccctgccggcacgcgacgtcgacgcggccgagctggcggaggtactggcgcggagcgccgccaccggcttcgacctggccgccgaactgccgctgcgcgccgcgctgttcgcgctcgccccggatcggcacgtgctgtcgctggtggtgcaccacatcgcgggcgacgcctggtccatggtgcccctgggccgcgacctgtccaccgcctacgcggcccgcctgcgcggcgaggagccgtcctgggccccgctgccggtgacctacgcggactacacgctgtggcagcgggacctgctgggggaggagcgcgacgaggacagcctcatcgcgcggcaggtggcgtactgggcccgtacgctggccgggctccccgacgaactcgccctgcccgccgaccgcccccgccccggcgtggcgagccaccgtggcgggaccgtgccgctgcgcctggacgccgccctgcacgtgcggctcgcggcgctggccgaggcccatggggtgacgctgttcatggtcctccaggccgggctggccgcgctgctgacccggcacggcgcgggcaccgacatcccgctcggctcgcccgtcgcgggacgcaccgacgacgccctggacgacctgatcggctgcttcgtcaacaccctggtcctgcgcaccgacacctccggcgacccgggcttcggcgaactgctcgcccgcgtacgggagacggacctggcggcctacgcccaccaggacgtgcccttcgaacacctcgtggaggtgctcaaccccaccaggtcgctgagccggcacccgctgttccaggtgctgctgacgctcaccggccggacggcggcgccggacgcgctgcccggactgcgggccgaggccgagcgggtggagtcgggcaccgccaagttcgacctgacgttcaacctgaccgaggccgtggatgccgggagcggccccggtggtgtcgaggggtacgtcggctacagcgccgacctgttcgagcgaggcaccgtcgaggcgcttgcggcccgtttcgtacggctgctggaggcagcggcggccgacccggacctgccgctgggcgccctggacatcctcggcgaggccgagcggcaccgcctgctgaccgagtggagcggcaccggcggccgtaccggtaccggagccgccgctgggggcgcccggtccaccgcgtcgctgccggagctgttcgagcggcaggccgcagccaccccggaggcccgtgcgatcgccgcccccgacgccgagctgtcctacgcgcaggccgacgcccgggccaaccgcctggcccgcctgctgatctcccggggcatcggcccggagcaggtggtcgccgtcgcgctgcccggctccgccgaactggtcgtcgcgctgctcgcggtgctcaagaccggcgccgcctacctgccgctcgacctggcgcagccgggggcccggatcgccgggctgctcgacgacgccacccccgcgctgctgctgacgaccagtcacgccgcgaccgggcttcccgatgacaccggcgcaggcaccggtactgacaccggccccgcccgccttctgctcgacgccccggacaccgtaagggcgctggagcggcagtccgcggcccgccccaccgacgccgaccggagcgccccgcagctgccggaccatcccgcgtacgtgatctacacctccggctccacgggccgccccaagggcgtggtggtcacccaccgcggcctcgcgcactacgtggcccgcgcccggcgcgtgtacacgggcttggacgggacggtcctcgtcccgacctccctggcctacgacctggccgtcaccggcctgtacggggcgctgacctccggcggctgcgcggtcgtggccgggctggacgacgacccttggctgacggcgctgctggcccgcaccccggcgacgttcatgaaggcgaccccgagccacctggtcctgctggccgcgctgcccgccgaattcgccccggtgcggcagctcatgctcggcggcgaggcgctgcccgcggcccaccttgaggagtggcgcgcacgccaccccgaggtggcggtcgtcaaccactacgggccgacagaggcgaccgtcggctgcttggacctgcatctggctcctggtgaagcgccgggcggcgccaccgttcccgtcggccggccgatggccggtatgcgggcgtatgtcctcgacgccgggctgcgtccggtgccgccgggcgtggcgggggagctgtacgtcgccgggccgcagctcgcgcgcggctacctcggccgtcccggcctgacggcggagcgcttcgtggcctgccccttcgcgccgggcatgggggtccccccggccgaaggctgggggaggatgtaccggaccggcgacctcgcccggtggaccgcggacggtgtgctggtcttcgcgggccgcgccgacgagcaggtcaagatccggggccaccgggtcgagcccggggaggtcgaggccgcgctggccggtcacccctcggtggcgcgggcggcggtggcggtacgggaggaccggcccggcgaccagcggctcgcggcgtacgtcgtaccggccggtgacgcggacggcgtggacccggcggagctgcgcgcgtacctggcgcggcggctgcccgagcccatggtcccggcgctgttcaccaccctgggcgcgctgccgctgacccgtaacggcaaggtggaccggcgggccctgcccgcccccgcgctggcgccggcgagcaccggccgcccgccgcgcagcccggaggaggagatcctctgcggcctgttcgccgaggtgctcggcgtgccccgggccggtgccgacgacgggttcttcgacctcggcggccactccctgctcgccacccgcctcgtcagccgggtgcggaccgtgctcggcgccgaactgcccgtacgggccgtgttcgagacgccgacggtggccgggctcgcggcccggctgaaggacgggtccggccgcaggcgccccccgctccgtccctttccccggcgcccggagacggtgccgctgtcgttcgcgcagcgccggctgtggttcctgcaccagctcgaaggggctcccaactacaacctgcccttcgtcgtacggctggagggcccgctcgaccgcgccgcgctcaccgccgcgctcggcgacgtggtcgcccggcacgagagcctgcgcacggtcttcccggacagcggtggtacgccgcggcaggccgtcctggacgccggtcctgccgccgcctccgtcgagctgccggtacggcacgtcgcggacgaggacgcactcgccgcgaccgtcgtcgcggcggcggcccaccgcttcgagctgacccgtgagctgccgctgcgggcagagctgtgcgcgctcggcgccgacgtgcacgtactgatcgtggtcgtccaccacatcgcgggcgacggctggtcgaacggcccgctggcccgggacctgtccgaggcgtacgcggcccgccggaccgggcaggcaccgcgctgggagccgctgccggtccagtacgcggactacaccctgtggcagcaggagttgctgggcagcgaggacgacccggacagcctggctgcccggcagatcgcctactggtcccgtacgctcgacgggctgcccgaggagctggacctgcccgccgaccggctccgcccggcggttcccggctacaccggcggagtggtgaccgtctccgtcgacgccggtctgcacgcccgcctggaggagctggcccgtacctcgggcgcgagcctgttcatggtgctccaggccggcctcgccgccctgttcacccgcctcggcgcgggcaccgacattccgctcggcagtgccatcgccgggcgcaccgacgacgcgctcgacgacctcgtcgggttcttcctcaacaccctcgtgctgcgcctggacacctccggcgacccggccttcgaggagctgctgggccgggtacgggagaccgcgctggccgcctacgcccaccaggacgtgcccttcgagcacctggtggaggtgctgaacccggcccgctcggccgcccggcacccgctgttccagaccatgctgaccctgcacaacaacgcccggccccggctcgacctgcccggactgcgagtgagcccggacatggccgaggtcggcaccgccaagttcgacctgtccttcgggctggtcgaggagcgcggcccggccggcgagcccgcgggcctcgccggcatcctggagtacagcaccgacctgttcgaccacgccaccgccgaggaactggtggcccgcttcgtccggctgctcggcgccgcggccgacgggcccggacggcgcatcggggagctggacatcctctccccgtcggagcgggtacggattctgggtgactgggccggtacctcgcaggacgcgcccgccctgaccgttcccgagctgttcgaacggcaggcgacgctgactcctgacgcgaccgccgtcgtcgcggccggtacgacgttgacgttcgccgaactgcgggcgcgcgcggatgggttggcccgtcacctggcagagcgcggcgtcggccccgacacgctggtcgggctgtgcgtgtcccggtcggcgtggatggtcccggcgatgctggggatctggaaggccggcggcgcgtgcgtgcctctggacccggactatccggccgagcggctggcgttcatggccgccgactccggggtgcgggtgctcgtcgctgagcccgacctggccgacgtgctggagggccaggccgcggaaaccgtccttctgggttcgccggacgcggacggcgagccgttcccgatgcccgccgtacgggccgaccacgccgccttcatcgtctacacctccggctcgaccggcgttcccaagggcgtgaccgtacggcacggcggcatcgcgaacctgttcgccaaccaccgcgagcgcgtcttcgccccggcggcgcgggctgcgggcggccggctgcgggcggcgcacaccgcgtcgctgtcgttcgacctgtgcgtcgggcagctcctcgccctcctcgcgggccacgaactgcacgtcgtcgacgaggacacccgccgcgacgccgtcgcgctgggcgcctacgtccgggaccacggcatcggctacctgtccatcaccccgaccctcacccaggaactgctcggcctcggcctgctcggcgcggaggggcaccagcccgccgcgatcctgcacggcgcggaggccaccggcccggggctgtggcagcggctgcggggcctgtccgccaccgaggtgcacaactactacgcgcccagcgagtgcaccgtcgacgcgatcggcgccaccgtctccggcagcgaacggccccgcatcgggcagccggtcgcgggcatggcggcgtacgtgctcgacgctgggctgagtccagtgccgccgggggtacggggcgagctgtacctggcgggcgcgcagctcgcccggggctacctcaaccggccggtcctgaccgcggagcggttcgtggccaaccccttcagcagcgtgccgggcgcccggatgtaccggaccggggacctagcgcggtggaccgccgacggcgccctggagtacgccggacgggccgacgaccaggtcaagctgcgcggtttccggatcgaactcggcgaggtcgaggcggtactggcgcggcacccggacgtggcccacgccgtggccgtcgtccgcgaggaccggcccggcgaccgcagactggtcgcctacacggtcgcggcacgcggcgccgtgccggacccggccgggctgcgcgcccacgccgcccggacgctgccggagttcatggtcccctccgccgtggtgccgctggacgcgctgccgctcaccccgagcggcaaggtggcccgccgcgccctgcccgcccccggctccgccgtaccccagggcggccgggagccgcgcacggcccgcgaagagatcctgtgcgggctgttcgccgaggtgctgggctgcgcacggcccggcgtcgacgaggggttcttcgacctcggcgggcactccctgctggcgacccggctgctgtcccgcgtccgcgccgtactggacgtggagctgacggtacgcgacgtgttcgaggcgcccacggtggccgccctcgccgcgcggctcgacgccggaggcgcggcgcggcccgccctgcgtcccgtcgaacgtcccggcacgctcccgctgtcgttcgcgcaacgacgcctgtggttcctcaaccggctggacggcgccggcgccgcgtacgccatgccgctcgcgctgcgcctgcggggggatctcgaccgggacgcgctcgccgccgccctcgccgatgtcgtcacccggcacgaagtcctgcgcacggtctaccccgagactgccggcacccccgagcaggtcgtactcgccccggagcgggccgcgcccggactgtcgtgccgcgccgtggccgagaccggcctcgcggcggcggtggccgccgcggcagcgcacccgttcgacctggccgccgagcccccgctgcacgcggaactgctcgccctcgaccccgacgaccacgtcctggtgctcgtactgcaccacatcgcgggcgacggctggtccaacctgccgctcacccgggacgtcaccgccgcctacgccgcccgccacgcgggcgaggccccgtcctggacgccgctgcccgtccagtacgccgactacaccctctggcagcaccggctcttcggcgaccccgccgacccggacagtttgatcgcccggcagaccgcgtactggaccgagacgctggcaggtctgcccgaggaactgccgctccccaccgaccggccgcgccccgccgtggcccgccaccggggcggccgggtgccgttcaccctggacgccgccgcgcacgccgggctcgccgacctcgcccgcgcctgcgacgcgagcctgttcatggtcggccaggccgcactcgccgccctgctcagcaggctcggagcgggcgacgacatccccatcggcacgccggtggcgggacgcaccgacgacgcgctggacgacgcggtcggctgcttcgtcaacaccctcgtgctgcgcaccgcgaccggcggcgaccccgccttcaccgacctggtggcacgggcccgcgaaacagcgctggccgcctacgaccaccaggacgtgccgttcgagcacctggtggagcatctggaaccggtcaggtcgctggcccgtcacccgctcttccaggtgatgctggcgctgcggaacaccggtgaggcgccgctgggcctgccggggctggcggtgagcgcggtgccggtggagaccacggcggtgaaattcgacctggcgttcacggtgagcgagcggaccggcccgcagggcgaggccgccgggctcaccggcgaactcgactacgcgagcgacctgttcgacgcggaaacggcgcgccggatcgccgcccgcttcgtgcgggtgctggaggcggtggcggccgacccggcacggaagctcgaagacctgccggtcctggacgcggccgagcgggagcgggtgctgaccgggtggaacgccaccgagcggcccctgcccccggtgacgctgccggagctgttcgcggcgcaggcggcccgtactccggacgcggtggccgtggccggcgagggcggcaccctgtcgtacgcggagctggacgcgtgggcggaccggctggcccgggccctcgtccagcggggcgcggggccggagcgggtggtggccgtggccctgccgcgttccgtggagctggtggtggcgctgctggcggtgctcaagagcggcgcggcctatctgcccgtcgatcccgggcaccctgccgagcgcacccgggcgatgctggacgaggcgcgtccggtgtgtgtcctggacgatccggagggcgtacggaacccccggccggagacggcgatcactgcgcctcccgtaccgctctccccgtcgcacccggcctacgtgctctacacctcgggctccaccggcacgcccaagggcgtcgtcgtcccgcacgcgggcgtcgtcaaccggctgctgtgggcgcaggccgagtacgggctggacgcctccgacgccgtgctgcagaagaccccgtacggcttcgacgtgtccgtctgggagttcttctggccgctgatcaccggcgcccggctggtcgtcgcccggccggacggacaccgggacccggcgtacctggccgcgctgatccgcgaacacgccgtgacgaccgtgcacttcgtgccgtcgatgctgcgggccttcctggacgagcccgccgccgcgcggtgcacgagcctgcggcgggtgctgtgcagcggtgaggcgctgcccgcacaactggccgcacacggccgggacatcttgggcggctcgaagctgcacaacctctacgggccgaccgaggcgtccgtcgacgtcacctcctgggcctgcgcggaccggccgggcccggtgccgatcggccgcccggtgcacaacacccgggcgtacgtgctggacgccgccctgcggccggtcccgccgggcgtgaccggggagttgtacctggccggtgtgcagctggcccgtggttacctggggcggccggggctgacggcggaacggttcgtcgcctgcccgttcgcgccgggcacgggggtccccccggccgaaggctgggggaggatgtaccgtaccggtgacctcgcgcggtggaccgcggacggagtgctggagtacgccgggcgcgccgacgaccaggtcaaactgcgcggcttccggatcgaacccggcgagatcgagaccgccctggcccggcacggcgatgtcgctcaggtcgccgtcgtcctccgcgaggaccgtcccggtgaccagcggctggtggcgtacgtgacctccgcgccgggagccgtaccggaaccgagggagctgcgcgagtccgtcgcccgcacgctgcccgcctccatggtgccggccgagatcgtcgtcctcgacgcgctgccgctgacgccgaacggaaagctggaccgccgcgccctgcccgccccggacgccaccgcccaggcccccggacgcgctccgcgcaccctccacgaagagatcctgtgcggcatcttcgccgacgtgctcggccgtcccctgcccggcatcgacgacgggttcttcgacctgggcggacactcgctgctcgccacccggctcgtctcccgcgcccgggccgccctcggcgccgaactgtccgtacgggacgtgttcgaggcgcctacggtggccggcctggccgcccgcatcggttccggaggcagccgacggcccgccctccggcccgccgcgcaccgcccggacgtcgtaccgctgtccttcgcccagcgccggctgtggttcctgcacaggttggagggccccagcccgacctacaacatgcccttcgcgctgcggctgtccggcccgctggacaccgccgccctgcgcgccgccctggacgacgtggtcgcccggcacgagaccctgcgcaccgtcttccccgagatcaagggcgctcccggccagatcgtactcggcccggacgagaccgccgtggagctgcgcacccaccgcaccgacgaggccgggctcaccggggcgctggccgaggcggccgggtaccgcttcgagctgaccgcagaaccgcccctgcgggccgacctgttcgccctcgccgacgacgatcacgtcctgctcctggtcctccaccacatcgccggggacggctggtcgaacgtcccgttcgcccgggacctgtcgaccgcctacacggcccggtgcgcgggccacgccccggcgtgggcgccgctgccggtccagtacgccgactacgcgctgtggcagcacgacctgctcggcagcgagcacgaccccgacagcctgatctccgcccaggtcgcccactggaccgaggcgctggcgggcctgcccgaggagctggcgctccccgccgaccgcccccgcccggcggtctccgcccaccggggcggacgggtggtcttcgcgctggacgcggagctgcacacccggctcgccgggctggcccgcgagcacggcgcgaccctgttcatggtcgtgcaggccggtctcgccgccctgttgacccggctcggcgcgggcaccgacatccccctcggctccgccgtcgccggccggacggacgcggccctggacgacctcgtcggcttcttcgtcaacaccctcgtgctgcgcctggacacctccggcgacccggctttcagcgccctgctggaccgggcccgcgagaccgacctggccgcctacgcccaccaggacgtgcccttcgaccacctcgtggaactgctcaacccggcccgctcggcggcccggcacccgctcttccaggtgatgctgacgctccagaacaacgccacgcccgagttcgccatggccggcctgcgggtgacgcccgacgtcgccgagctggccgcggccaagttcgacctgctgttcagcctcaccgaccactacaccgaggacggcgcccccgaccgggtcgacggagtgctggagtacagcgccgacctcttcgaccacggcaccgccgaagccctcacagcacgcttcgtccggctgctgtgcgccgtggcggaggccccggaccggccgatcggcaccgtcgacctcaccgacgccgccgagcgcgagcggctgctggagacatggaacggcgccgccctcaccacgcctccggtgacgctgcccgaggcgttcgaggcgcagacgcggcgcacgccggacgcggtggccgtcaccgacggctccacaaacctcacctacgccgcactgaacgcgcgggccaaccaactggcgcggctgctcctcgggcgcggcatcggcccggaacagcttgtcgcggtcgccctgccgcgctcggccgagctggtggtggcgctgctggccgtcctcaagaccggtgccgcctacctgcccctcgacccggcgcacccggccgaacggctgcgcttcctgctcggcgacgcccgcgccgcgtacgggatcaccggctccgccctggtcggggacctgccggagacggacacccgctggctcacgcccgacagcgacgaggcgcggcgggaagccggggaccagccggaacacgacgtgacggacgccgagcgcaccgcgccgctgcgcccggagcaccccgcgtacgtcatccacacctccggctccaccggcacacccaagggcgtactcgtcgagcaccgcgcgctcgccgcctacctggcgttcgcccgcgaggcgtaccccggcaccgcgggcaccaccctgctgcactcgcccgtctccttcgacctcaccgtcaccgcgctgtacgcgccgctcaccgcgggcggccgggtacggatcgcggaactctccgccgacgcgccggccccgggcccgtacaccttcctcaaggtgacgcccagccacctcgcgctgctcggtgccaccccgcccggcccggagacggcggtcgcggacctcgtcgcgggcggcgaacagctcaccggtgaacagctcgcgccctggcggcgggcgcagccgcaggccgccgtggtcaacgagtacgggccgacggagacgaccgtgggctgcgtcgtcctgcggatgccgccgggcgccacggccggggacggcccgctgccgatcgggcggccggtgcccggcacccgggcgtacgtgctggacgagcggctgcggccggtaccgcccggcgtcacgggcgagctgtacctggcgggcgcgcagctcgcacgcggctacctgaaccggcccgcgctgaccgccgagcggttcacggccgacccgttcggtgggccgggcgggaggatgtatcgcaccggcgacctggcccggtggtccaccgagggcgaactgcacttcgccggccgcgccgacgagcaggtcaaggtgcggggccaccgcatcgagccgggggagatcgaaaccgcgctgctgcgccacccggccgtcgcccgggccgccgtcgtcgcacgggaggaccggacgtccggcacccagctggtggcctacgtcgtcccggcggacggcgaacggagcggcgacgcggccgcgcgggcccagctcgacgagtggcaggaggtctacgactccctgtacgggggcgacggccaccaggacgcggagcccgcgccgttcggcgccgacttcaggggctggaccagcagttacgacgggcggctcctgccggtcgagcagatgcgccgatggcgggaggccaccgtcgaccgcatccgcgaactgcgcccgcgccgggtgctggagatcggcgtgggcagcggactgctgctctcccggctcgccccggactgcgagacctactggggcaccgacatcgccgccgacgccgtcgaccggctgcggacccaggtgcccgagcggccggacctggccggacgggtggaattgcgcgtacagcccgcggacgacctcaccggactgccggaaggtttcttcgacaccgtcgtgatcaactcggtcgcccagtacttcccggacgccggctacctcaccgacgtgctcggcacggcgctgggcctgctcgcacccggaggcgctgtcttcctcggtgacgtgcgcgacctgcggctgctgcggtgcatgcacacggcggtgcagctgcgccgcgccacgccgtccgccgaccccgccgccgtacgccgcgccgtggaacaggccgtcctgctggagaaggaactcctgctcgaccccgcgttcttcgccgcgctggcggcacggaacccggacgtcgccggcgtcgatctgcgggtcaagcgcggcgcgtacgtcaacgagctgaaccgctaccggtacgacgtcgtcctccacaagcggtccgccgacggaccgacggacctggcggccgcaccggaactgcgctggggccacgacgtgaccggacccgacgacctcgcccggcgcctcaccacggagcgcgtgcccgcgctgcgggtgaccggggtgcccgacgaacggctggcggctgaagccggtgccgcgcgcggactggacgagggacggccgctggccgacgtcctcgccctcctcggccccgccgagcagccgaccgcgccggatctcggtccggacaccttccacgagctgggcgaacgcgccgggtaccaggtcgcggtcacccgctccggcgatgacgccgaactcctcgacgtgctcttcaccgatccggcggtgaccgccctcagcggcgtccaccgcccggcggccgagccgtcgcacccgctcgccaacgacccggcggcctcccggcagcacggcgaactggtcgcctcgctcaccgcgtcgctgcgcgcggaactgcccgctcacatggtccccgccgccgtcctggtcctcaccgaactcccgctgaccggcaacggaaagctcgaccgggcggcgctgcccgccccctacctggggtccgtcgagggttcgcgcgggccctcgaccccgcgcgagcgagtgctgtgcgacctgttcgcgcaggtactgggcctggcggcggccggcgtcgacgacaacttcttccacctcggcggacactcgctgctggcgacccggctgatcagccgaatccgtaccgccctcggtgtcgaactgcccgtccgcgccctgttcgagacacccacggtcgccggactggccgcgcggctggacgacgaggagagccgggacgcgacacggcccgctctgcggccgatgtcccggcccgacaccctgccgctgtcgttcgcccagcagcggctgtggttcctgcaccggctggaaggcccgaacgccacctacaacatgccgatcgccctgcgcatgcgcggcgccgtggaccgtacggcactgcgcgcagcgctggccgacgtggcggcccggcacgaaagcctgcgcactgtcttcccggtgacggacggagtgccccggcagctcgtcctcgacgcggacggcgcgcgccctgaactgctcgtacggcacaccgacgagaccgggctgcgcgaggccgtccaggcggcggcgcgctaccccttcgacctcgcggccgacctgccgctacgggccgaactgttcgacctgggcgagcgggagcacctgctggtgctggtggtgcaccacatcgcaggcgacggctggtcgatggcccccctggcgagggacctggccaccgcctacgccgcgcggacccggggacgggcgccgtcgtggacgccactgcccgtccagtacgccgactacacgctgtggcagcgcgcgctgctcggcgacgagaccgacccggacagcccgttcgcccggcagatcgcctactggagcgagaccctggcggacctgccccaggaactcgcgctcccgacggaccgtccccggcccgccgtgtccacctaccgcggcgagatgctcggcttcgacctgagtcccgagctgcaccgtcgcctggcggacctcgcccgggacaccggaaccagcctgcacatggtgctgcacgccggcctcgccgccctgctgacccggctcggcgcgggcaccgacctgccgatcggcagcggcgtcgccgggcgccacgacgaggcgctcgacgacgcggtcggactcttcgtcaacacgctcgtgatgcgcaccgacacctccggcgacccgtcgttcggcgaactgctgcggcgcgtacgggagacggccctgaacgcctactcacaccaggacgtgcccttcgagcacctggtggaggcgctcaacccggtccgctccccgaaccggcacccgctgttccaggtggcgatggtgctccagaacaccccggaggccggactggcgctgcccggcgtcgaggtgacgcccgaaccggccgcctccgggacctcccggttcgacctgttcttcagcgtcaccgaacgcggctcttcagggggcgtgcgcgggacgctggagtacagcaccgacctgttcgaccggcggacggccgagacgttggtggagcggtgggcacggctgctggaggcggtcaccgccgacccggcgcgtcccatcggtgaggccgacgtgctcaccccggccgagcacgcccgtacagcccgtgcatcgggcgccgacggtccgcaggtgccgcccgccaccgtgcccgagctgttcgcacgccaggtggcccggcaccccggagcccttgcggtgctgtccgattccggcacgctgacctaccaggagctgaacgtccgggccaaccgcctggcgcacctgctgctgtcgcgcggcatcggtcccggacacgtcgtcgccctggcattcccgcgcggcgccggcctgatcgtggcgctcctcgcgacgctcaaggcgggtgcggcctacctccccctcgacaccggctacccggccgaacgcctggccttcatgctcgacgacgcccgcccgtcgctgctgctgacgggtgtcgcgggccaggagatacccggctccggccgcctgtcccggctcgacctggacgacggcgacgggcacctggccgggcagccggacaccgaccccggcgccgagcaggccggacggcccgaccccgcacacccggcctacgtgatctacacctccggctcgaccggcgtacccaagggcgtggccgtcacccacgcgggcgcggccgccctggcggcgacgcaggcggactccttcggcgccgggccgggcagccgggtcctccagttcgcctcgccgagcttcgacgcggcgttctgggagctgtgcatggcgatgctgaccggcgccgccctggtggtggccccggccgaacggctcctgccgggcccggacctcgccgccgtactggccgagcacgaggtgacgcacctgaccgtgccgcccgtggcgctgggcgtcatgacgcccgaccagctcgcaccggtgacctgcctggtggtcgcgggagagagctgcccgccgcagacggcggcggccttcgccgccggccgccggatggtcaacgcctacgggccgaccgagtccacggtctgcggcacgatgagccggccgctgaccggtgacggcgccgtcgcgcccatcggcacccccatcaccggcacccgcgcgtacgtcttggacgcggcgctgcgtccggtgcccaccggcgtggcgggcgagctgtacctggcgggcgcgggcctggcccagggctacctgggacggccgggagtgaccgccgagcggttcaccgccgacccgttcggcgcgccgggcgcccggatgtaccggaccggggacctcgcgcggtggaacgccgaaggcgagttggagttcgccgggcggaccgacgaccaggtcaaggtccgtggccaccgggtcgaactcggcgaggtggaggccgcgatggccgcccaccccgaggtggcgcaggccgtcgcgacggtccgcgaggacggcccgggcggtacccgcctgctcgggtacgtggtcccggcggcggacggcccgcgattcacggaggccaccggcgccgccgaggccgcgcaggccgccgacggcgcgccggaacgggccctcggcgagtggcggcagctctacgacaccctgtacggcacgacgcagaccgccccgttcggcgaggacttcaccggatggaacagcacctacgacggggaacccctgcccgccgacgagatgcggcagtggcgggacgccaccgtcgcgcgcatccgggacctgcggccccggcgggtgctggagatcggtgtcggccgcggcctgctgctgtcgaagctcgccggggagtgcgcggcgtactggggtacggacctgtcggcggccgtgatcgagtcgctgacggcacaggtcgccgaacggcccgcgctcgccggccgggtggagctccgcacgcagccggcggacgacctcaccggactgccggaaggtttcttcgacaccgtcgtcctcaactcggtcgcccagtacttcccggacgccgcctacctgaccgacgtgatcggcgccgcgctgcggctgctgacgccgggcggcgccctgttcctcggcgacctccgcaacctgcggctgctgccgagcctgcgggcggccaccggcaccaccgcacccgagcgggaactgctggtcgacccggacttcttcccggccctgcccgccaccgtccccgagatcggcgccgtcgacctgcgcctcaagcgcggggcgtaccacaacgaactgtcccggtaccgctacgacgtcgtcctgcaccgggccccgctgaccgcaccggcccggccgttcgccgacgcacccaccctgccctggcacccgggcctgggcggaacggcggagatcgccgcgctgctgacggaacggcagccggaccggctgcgcctcaccggcatccccaatgcgcggctggcgggcgaggcggccaccgggcacctcgtgaccgtggaccccgagacgctgtgcgcgacgggggagcggctggggttctggacggccgtcacctggtcgggcacggccggtgacgactgcttcgactccttgttcgccgctcaggacgccgccgcaccggacgacaccgccccgtacggcagtgccccggtcaccggggtgtacgtgccggcggacggccgcaccgcaccgccgtccttcaccaacgacccggcggcggccgtcgcgccgtcggcggccctgggctcttcggtacgcgcctggctgcgccggcgcctgccggagtacctggtgccctcggtggtcattccgctcgacgtcctgccgctgaccccgagtggcaaggccgaccgtacggcgctgcccgcgccggagacgacggccgggaccacggcgggccgtcctccggcgaccgcccgggagcggctgctgtgcgacatcttcgcggacgtgctcggccttccgtccgtcggcgccgacgacgacttcttcgacctcggcggcgacagcatcgtctccatccaggtggtcagccgggcccgggccgccggtatggcgatcggcccgcaggacgtgttccggcacaagaccgtggcggccctggccgtcgccgccggaacctcggacgcggcgcccgccgccacggaggcaccgcaggcgggcatcggcgtggtgccgctgacaccggacctgcacgccctgcgggagcgcgacgccgaccccggcaccgggtacgaggcgatgaccgtacccgccccggccgggctcggcgccgaagcgctggaggccgccctccaggcggtcctggaccaccacgacatgctgcgggcgcgcctgacctctggcgggcccgtggcggcggacgggccggggtggctgctcgaagtccggccgcccggcaccgtccgggccgcggaccatgtgcgccgcaccggcaccgcaccgcacgaggccgccgtggccgaggaagagcgggccgccgcggcccgcctggcgccggagtccggggtgctgctccaagccgtccacttcgacgccccacccgggagccccggcttgctgctgctcgtcctgcaccgtctcgtcaccgacccgcagacctggcgcatcctgctgcgggacgtcacggacgcctgcgaggcagccgctgacgggcggcagccccgactcgccccggtcggcacgtcgttccggcgctgggcccagcacctgcgcacccaggcacgggacccccggcgaaccggagaactgtcccggtggaccggcatgctcgccggacccggcagcccggacgatgccccggacgcgcgggcgaccgggaacccggacggcatcccggcgcacgaagccggggacgccatgctgacgaccctcctgtccgccgaacgcaccgggcaggtgctcaccgacgtaccggcggccttccacgccgacgccgaggacgtcctgctcaccgcgttcgcgctggcggtcgccgattggcagcgccgcagcagcgccgccgacggcaccgtgctcgccgacgtacggcgcccgggccgcgtaccgctcggcgagggcatcgacctgtcccgcaccgtgggccgctgcacgaccacccacccggtccgcctggacctgggcgacccgggcggggaagcgctgcgggaggacggcgggcacgccgccgcgacggctctcaaacgcgtcaaggaggaactgcggtccctgccggacggcggtctgggccacggcctgctgcgccacctccaccccggcaccgcccccgaactggcggccctgcccgtaccccggttcgccttccactacctcggagagctgccgcaggccccgtccggcccggacccggccggcgaaccggcgtgctactgggccgaggtggccatcactgccgtggacggccccggcgggccacggctggccgcgacctggcgccgggcccacgcgcccctgaccggccgcgacctgaacgacctggccaccgcctggacccgcgcgctcgaaacgctggccgcccaggccgcccgccccggcgccggtggccgcaccccgtccgacgtaccgctggtccggctcagccaggacaagctcgaacagctcgaagccgcatggaggcgatcacagtga

SEQ ID NO: 23 - DNA, 7131 bp, Streptomyces sp.
  source 1..7131: mol_type = genomic DNA
gtgacgcagccggaactggaggacatcctccccctggcaccactccaggaggggctgctgttccacgcgctgtacgacacggcagcgctcgacctctacaccttgcagagcgcctacgacctggacggggacctcgacgcgacggcactgcgcgaggccgcgcgcacgctgctgcggcgccacgccgccctgcgcgccggcttctggcacgagggcctggacgaaccggtgcagatcgtcccgcgcgaggtcgccctggcgtggcagcaggccgacctcaccgggaccgcccccgaggagcgggaggcggaggcggagcgcatcgcggccgaggaccggctccgccgcttcgacccggcccgcccgcccctgctgcgcttcacgctgctgcggctcgggacggaacggcaccgtttcgtcgtcaccgcgcaccacttggtgctcgacggctggtccgtacaactgctgctggaggagctgttccagctgtacgtccggggcggccgggaggacgggatgccgccggtggcccgctaccgcgactacctggcctggctggccggacaggaccgggccgccgccgaggacgtgtggcgccgggcgctcaagggcatcgaggagcccacccgtgtggcgcccgccgacccgtcccgggaaccggccctgcccggccagatcctgcgcgacctgccggacggcctcgccaacgcgctggacgccctcgcccgccgcgaacggctgaccctgagcacggtcttcctgggcgcctgggccgtcgtcctcggccggctgaccggcaaggacgacgtggtcttcggcaccaccgtctccggccgcccgccccaggtgcccggcgtcgagcggatgatcggcctgttcgccaacaccctgccggtacgggtccgcctcgacccggaacagccgctgaacgggatgctggccaggctccaggaagaacagacacacctgctggcctgccagcacgtggggctctccggcatccagcgcacgctcggctcgggcgaactgttcgacacggtgaccgtgttcgagaacctgcccgccgggcagagcggactcgccgccaccgcgcacggcggcggaccgcgggtgacggcgagccggagccagggcgtcacccactacccgctcaacttcatggtgatgcccggccgcccgctgcggctgcggctggaccaccagaccgggctgttcgaccgggacgccgccgaggcgatcatggcgcgggtggtccgggtcctggagaacctggtcgcggaccccggccggcgcctcggcgagctggagatcctggccccggccgaacgggcccgggtgctgggggagtggagcggcacggccggagagccgccggccgagctgatgccgcaggcgttcgcggcccgggtggcgacgaccccggacgcggtggccctggtctgcggcgaggtgtcctggacgttcgccgaactggacgcgcgcgcggaccgcctcgcgcggcgcctggccgcccggggtgccggacccgacgccctggtggggctgtgcgtgcgccggtcggcggacatgatcgccgccgtgctggccatctggaaggccggcggagtgtgcgtaccgctggaccccggctacccggcggagcggctggcgttcatgctggccgacgcggccgtacccctggtgctgaccgaacgggagctggccgggcgcctcggggaggaccgggcggcgtccttggtgctgctggacgacgacaccccgtccgaaaccggtgttgccctccgtacgccgcccctgcacggcgaccacgccgcgtacatcgtctacacctccggctccacaggcgtgcccaagggcgtcgtcgtctcccacctgggcctggcgaacctgtacgccgaccaccgcgaccgggtgttcgccccggccgccgacgccgtggccgcgtcggccggacggcggctgcgcgtggcccacacggcgtcggtgtcgttcgacgcctccatggggcaactggccggcctcgtcgccggccacgaactgcacgtcgtggacgaggacacccgccgcgacgccaccgcgctgctggcctacgtacgcgagcggagcgtcgactacctgggcatcgccccctcgctgatgcgggaactgaccgcgctcgggctgctggacgggcccggacaccgcccggcggtgatcctcctcggcgcggaggccgtcgacgcggccctctgggaggcgctgcgggcccacccggagaccgaggtgcacaactactacgggccgagcgagtgcacggtggacgtcgtcggcgccctggtacgcgacagcgcccggcccgtgatcggccggccggtcgccaacacccgggcctacgtgctcgacgcacggctgcgccccgtaccgcccggtgtggtgggcgaggtctacctggccggcatccagctcgcccgcggctacctgggccggcccggcctgaccgccgcacgcttcgtcgccgacccgttcggtacgccggggacgcggatgtaccgcaccggagacctcgcccgctggacggccgacggccggctggacttcgccgggcggaccgacgaccaggtcaagctgcgcggctaccgggtcgaacccggcgagatcgcgacggtgctggcccgccacgccaccgtcgcgcacgcggccgccgccgtacgcgaggaccggcccggcgaccagcgcctgaccgcctacgtggtgccggcgcccggcgcccgggcgcacgcggaggaactgcgcgagttctgcgccgggacgctgcccgagttcatggtgcccgcccagatcgtcgtgctcgacgccctgccgatgacccccagcggcaaactggaccgccgcgcgctgcccgcccccggcgcgcgcaccaccggcggccgggagccgcgcgacccggtcgaggacatcctgtgcgggctgttcgccgaggtcctcggcgtggaccgggtcggcgtcgacgacggcttcttcaccctcggcggccactccctgctcgcgacccggctggtctcgcgcgtccgcgcggccctcggcgcggaactcgccgtacgggacgtcttcgaggccccgaccgtcgccgggctcgcggcccggctcggcaccccgcgcgaggacacccggcgacggccgccgctgcggcccgccggcgagcgcccggccgaactgccgctgtccttcgcccagcggcgcctgtggttcctgcaccagatggaaggccccacgccgacctaccacatgccgttcgcgctgcgcctgaccggcgggctcgacgtcgcggccctgcgcgccgccctgaacgacgtggtggcccggcacgaggtactgcgcaccgtgtacccggacaccgggggcaccccgcgccagcacgtactagggcccgccgaagcccccgcaccgctgcccgtgcggacggtcgccgaggacgccctggcggcggccctcgcggacgcgtacgcgcgccccttcgacctcacccgcgaaccgcccctgcgcgccgaactgttcgcgctcggccccgacgaccacgtcctgctgctcgtactgcaccacatcgcgggcgacgcctggtccaacctcccgctcgcccgcgacctgtccgccgcctacgcggcccgccgtacaggcgaggccccggcctgggcgccgctgccggtccagtacgccgactacaccctgtggcagcgcggcttcctcggcgaggagagcgacccggacagcgcgctctcccgccaggtggcgtactggcggacggcgctcgacggactgcccgccgaactggaactgcggcacgaccggccccggccggaggtgtccgaccaccgtggcgggacggtgccgctgcacgtcggccccgccacccacgcccggctcgccgggctggcgcgggcgcacggcgcgagcctgttcatggtcgtccaggcggccctcgcggcactcctgacccgcgagggcgcgggcaccgacatcccgatcggcacaccggtcgcggggcgcaccgacgagggcctggacgatctcgtcgggtgcttcgtcaacaccctggtgctgcgcaccgacaccgggggcgacccggtcttcacggacctcctcgaccgggtccgccggaccggcctggccgcgtacagccaccaggacgtgcccttcgagcacctggtggaggtgctcaacccggcccgctcggcggcccggcacccgctgttccaggtgctgctctccctccaccacgccgcggacctgccggtggagctgcccggactgcgcgtccgggccgaggagacccggtcccgctcggccaagttcgacctgacgttccggctggccgagcggaagggcccggacggcgggcccggcggcctcgaaggcggtgtcgagtacagcaccgacctgttcgaccggagcacggtggagggcctggccgcccgcttcgcgcggctgctcgacgccgtcgccgccgaccccggccggcggatccggcggctcgacgtcctgtccccggccgagcgggagcgggtgctgaccgggtggaacgccaccgagcggccgttgcctccggtcacgctgccggagctgttcgcggcgcaggtggcccgtactccggacgcggtggccgtggtcggtgagagggacactctgtcgtacgcggaactcgacgcgtgggcacagcggttggcgcgggctctgaccggtcggggggcggggccggagcgggtggtggccgtggtgctgccgcgttcggtggagctggtggtggcgctgctggcggtgctcaagagtggcgcggcctacctgcccatcgaccccgccctgcccgccgaacgcatccgcctcctgctcgacgaggcgcgtcccgtgtacgtcctggacgatgcggacggcgtgcggaacccccggccggagacggcgggtaccgcgcgtcccgtaccgctctctccgtcgcacccggcctacgtgctctacacctcgggctcgaccggcgcccccaagggcgtcgtcgtcccgcacgcggccgtcgtcaaccggctcctcggcatacaggaggagttcggcctcgaaccctccgacatctgcctgtacaaggcaccggccgccttcgacgcgtccgtgtgggagctgttctggccgctgaccaccggcgcacgcatcgtggtggccgctcccgacgggcaccgtgacccggcgtacctggccgcgctgatccgcgaacacgccgtgacgacggtgcacttcgtcccgtcgatgctgcgcgccttcctggacgaacccggcgccgcccgctgcacgagcctgcggcgcgtccagaccggcggcgaaccgctgcccgccgagttgcgtgaccgtgccacccacctcctcggcgcgcccctgaaccatctgtacggaccgacggaaaccaccatccacgccacgacctggtcctgcgggcggccctcggccgccgcaccggacaccactgccgcccgtccggtgcccatcggccggcccgtccgcaacacccgggcgtacgtgctggatgcggcgctcggtcctgtgccgccgggtgcgacgggggagttgtacctggcgggtgtgcagctggcccgtggttatctggcaaggccgggcctgacggcggaacggttcatcgcttgcccgtacggcgcaccgggggagcagatgtaccgtaccggtgacctcgcgcggtggaccgccaacggggtgctggagttcgccgggcgggccgacgaccaggtgaaactgcgcggcttccggatcgaactcggcgagatcgaggccgccctgtcccggcacgaggacgtcgccgcggcggccgtcgtcctccgtgaggaccgtcccggtgagcagcggctggtggcgtacgtcgtgcccgcgccggaaaccggaccgcaccccgccgacctgcggcgccacgccgcgctgcacctgccggagttcatggtgccggccgccgtggtggtcctggccgcgctgcccctgacctccagcggcaagctcgaccggcgcgccctcccggagccgggcggcccgctcaccgtgaccgccaagcggcccgcccgcaccccggaggaggaggtgctctgcggcctgttcgccgaagtcctcggcctgaccggaaccgggatcgacgacgacttcttcgacctcggcggggactcgctggcggccatccggctggtccgccgcatccgggaggcggcgggcgtggagctgtcggtcagcgacctgttcgcggcgcccacggtcagccgcctgatggaccggctgcgcctcggcgtccggggcgacgccttcgacgtactgctcccgctgcgcgaatccggcgggctgccgccgctgttctgcgtccaccccgcaggcggcctgagctgggcctacaccggattgctgcggcacctgggccccgaccggcccgtgtacggactgcaggcccgcggcctcgcccgtcccgagacgtacccgacctcaatggacgagctggtcaccgactacgtcaggcagctccgctcggtccggcccaccggcccctaccacctgctgggctggtccttcggcggcctcgccgcccacgcgatcgccgtccgcctccaggccgagggcgagaaggtcgccttcctcgccctgctcgactcctacccccgcgacccggaggccgcggtcccgcccgtggaggagcgggacgtcctggcgacgctgctcgacttcgcgggccacggcgaggacgtcaccgacgccgaacagctgcgcgtcgaggacgtcatggagatcctgcgccgcggcggcgggcccctggcggacatcgaggaacgccgcgtgggggccctggccgacgtcttcgtcaacagcgcggaactcgcgtcggcgcatgcgcccggccggttcgacggcgacctcctgcacttcaccgcgaccgccggccgcccggacggagcgcccacggcggccgtctgggagcccttcgtcaccggacgcgtacgccggtacaccgtggactgtacccaccacgccatgtgccgccccgagccgctggccgagatcggccgggtggtcgccgagcgcctggacggcgcggaccggccgaggcggcaggacggccccaaccggtaa

SEQ ID NO: 24 - DNA, 231 bp, Streptomyces sp.
  source 1..231: mol_type = genomic DNA
atgaccaatcctttcgagaacgccgacagcacctacctggtgctggtcaacgacgagggccagcactcgctgtggccgtcgttcgccgaagtcccggccggctggacggtcgcgcacggcgaggacacgcgggacgcctgcctcgcccacgtcgaggagcactggaccgacatgcgcccgaagagcctcgtccgcgtcatggacgaagccggggcgccgtccgcccgttga

SEQ ID NO: 25 - DNA, 1116 bp, Streptomyces sp.
  source 1..1116: mol_type = genomic DNA
ttgaccgccgcggaccgcgaggagagagacaccgctgtgctgacgtaccaggaaccccccgccggcggcggcgcctgggccgagacggtggagctgacgccgcaggaagccgagcagttccgcaaggtcgccgacgagatcaccggcatgctgccgcacgggaagccggccgggcccggacagctcgacgaccccgggctgctcgcggagatcgaggtggcggcgcgctggctcccgccgcgcctggcccgcgcgctcagccgcttccgcaggcagggcaacccgcacggcgtcctgctcgtacgcaacgtaccgctcgacgccgacctgcccccgacccccgccgacggcgccgcacccgtctggtcgcggctcgggaccgcgaccgtcggccagctcgccgtggtctcctggctgggcgacgtcatcgcctacgccgacgagaaggccggccggctggtccaggacatcgtcccggtcagcggcgccgaggagtgccaggagaacagcggcagcgtgctgctcgaactgcacaccgaggacggcttccacccctacaaaccggacttcgtgacgctgctgtgcctgcgcgccgaccacgagcgccgggcccgcaccacctacggtgcgatcacccgggcgctgccccgcctgtccgccgagtgcgtccgcgtcctgcgccgcccgctgttccgtatccacttcagcagttccttcggggggccgggcgccggggcgaaggggagcggtacgggtactggtaccggcgtggtctcgccgccgctgcccgtgctgtccggaccggacggtgacccggagatcgtcgcggacttccacgccatggaacccctggacgagaccgcggcgcgcgccttcggcgaactgcgtgacgccatgctcgccgtcctcgacggcgcggtcctcgaagccggcgacctgatcgtcgtggacaaccgcaccgccgtgcacggccgcaccgccttccggccccgctacgacggacaggaccggtggctgcggcgctgcttcgcggtcgccgacctgcgcccgtcccgcgccgcccggaccccgggctcccaggtgtgcgcgccgctgaagccggagggcgggccgtga

SEQ ID NO: 26 - DNA, 171 bp, Streptomyces sp.
  source 1..171: mol_type = genomic DNA
gtgacggcggaccgccgcgccgtcgccccggcggccgcgggccgggcgcccggcagacaggcgtccggctgtccgacgcccggcagaccgaccagcactccggaagcagcagtgatcggcacggacatgagcgcgcaggtccgggcgctggccgcgaagggtggacggtga

SEQ ID NO: 27 - DNA, 363 bp, Streptomyces sp.
  source 1..363: mol_type = genomic DNA
atggagcccatggaacccatggccgggaacgatccgcacattcacgtccaggcacaggcgggccgcgacgacgccgctgcccgcaacctgatagccgccacgttcggcctggtcagtaacctgccgagcgtcgtcgccacgggatgcgggctgcgggtgccgtacgcgatgacctcgtcgcgtccggaccgtgtcacctgcctcgcctgccgtgagcacgcacggcaggcgcacctgcgcttcgcggtggaggtggaacgcctgagccgcatgctgggatcggtcatcacccccgcccagggcaagttggccgccgaccggcaccgcgacctcgcgcggcggttctccgacgacgagggctga

SEQ ID NO: 28 - DNA, 390 bp, Streptomyces sp.
  source 1..390: mol_type = genomic DNA
atgagaatctacgtgaccagcgtctttgtcgacgaccaggccaaggcgctgagcttctacaccgacaagctgggcttcgtgaagaagcacgacgtcccgctgggcggagaggcccggtggctgaccgtggtctcccccgaagacccggacggcaccgaactgctgctggagccctccggccacccggcggtgcagccgtacaagacggcgctggtccaggacggcatcccggccacctccttcgccgtggacgacgtgcgggcggagttcgagcggctgcgcgggctcggcgtgaccttcacccaggagcccctggagatgggcacggtcaccaccgcggtcctggacgacacctgcggcaacctgatccagatcgtgcacaccgcgtag

SEQ ID NO: 29 - DNA, 294 bp, Streptomyces sp.
  source 1..294: mol_type = genomic DNA
atggcagaggatgttttcaaggcgctggccgaccccacccgccggcgcatcctcgacgagctggcggagcggaacggtcagacgctgttcgagatatgcgcccggctggtgaccaagcacggcctggggctgtcccggcaggcgatcagccagcacctggccgtcctcgaatccgccggcctggtccgcacccggcgccagggccgctacaagttccacgacctggacaccgcaccccttgagcacatcatgacccgatggctcagcaccgacacgccggagagcaccccatga

SEQ ID NO: 30 - DNA, 693 bp, Streptomyces sp.
  source 1..693: mol_type = genomic DNA
atgcacccttccgtgcggaacaatgcccggatggaagatcgaatagccccgacccccctgcttcccgtccgcggagcggcccgctgcgccgtggccgccctcgtcctcgccggtgccgcctgggcggcccgcgccctgtggcagatccggctcgccaccgccgggatgccggcttccggaccaccggaccaaggcgaagggaagcaccgcccgctgaccgcgctggaggacggctaccacatcgtcaccactgtgggcagcgcggccacggtgctctgcgcgatcaccttcctgatctggctctcgcgtgtccgggacaacgcccgcgccctctccggacagtcgccccgctacgccaggccgtggctctacgcgggctggatcgtgcccgtcgcgaacctgtgggtgccccgtggcgtcgtcgccgacatccaccgcgcgagcgccccgggagaacggctgccgcacgccgtgaactggtggtgggggctgtggctcgtcggaatgctgagcgggacgggcctgatgtacaccgtccccacggacgacgtcatcgaacgtgcctaccaggcggtgtatccgctcctggccgccgacgcggccgtcgtcgcggcggcggtggccggtgtcttcgtcgtccgcgccctcaccgcggcacagcggcggcgcggcgtcgattccgtacgggcctga

SEQ ID NO: 31 - DNA, 1014 bp, Streptomyces sp.
  source 1..1014: mol_type = genomic DNA
gtgcatcagctcaccggcgcggccggttcggccgtgtcggaggcgacgtccgcggcgtactacgcgtcgatcgcacaggccgaggagagcggcgacggcacggtgcggcaccggctgcgtccggaggtcggcgacggcaccatcgaggtgacgtcgctgcgcagcgggttcgacatcgtccggtacgacgtcgcgttcgccggcggacaccaggtgggattccggttcccgggggaccacttcgagctggagcactgtgtcgacgggcgcatgcacatcagcgaggccggggccggcgccggtgacctgcgcgcccgctcggtggcgctcagccctcgcggcgccatcgacggcgtcgtggcgcacggcgacggtgagcggtaccgcgcggtgtcggtcaccggcacctggtccgggctggagtcctacctcggcggcctcggcgcgggcgccctgccgcccaccggaccggagagcgtcgcccgcgagcggtacctgggccggtccgccgcgctgcgcccggccgcgggaccgctggaggagatcttcctgcaccggcgctcctcaccggggcgcctgctgttcatggagtcgcggctggtggcggcgctcgccgcgctgatcgacgggctcgcgtccgacgcgtcgcccgccgacgcccgggaccgcgccgcgccggccctggccgaccacgagacggccgcgctgcgccgggtgcccgaactgctgtggcgggagcgccacgcaccaccgacactcgccgagctggcccgggcgtcgtcgatgtcggtacggcgcctgaccaccggattccgcgcgctgtacgggacgtcggtgatggagtaccaccggcggcactgcctggaccgggccgccgcgctgctggtcgagaccgactggccggtctcccggatcgcgcacgagatcggctacgcctcggccggcaacttcggctatgccttccggcgcgaccggggcacgtcgcccgcccggttcaggcgggcgcactcctga

SEQ ID NO: 32 - DNA, 1185 bp, Streptomyces sp.
  source 1..1185: mol_type = genomic DNA
atggtcctcggcgggctgtcggcggtctcaccgttcgccatcgacatgtacgcgcccggcttcccggagatcgtcacgtccttcgggacctcgaagacggccgtccagctctccctgacggcgtgcctcgtcgggctcgccgtcggacaggtcgtcctcggcccggtcagcgacgcgacagggcggcggcgcctgctcctggccggttccggcctgttcagcgtgctgtccctggtgtgcgcggcggccccgtccatcgccgtgttcgacgcggcccggctgctgcagggggtcgcgggcggcgcgggcatcgtcatcgggcgggcggtggtcagcgacctgttcaccggcgcccccgcggcccgccggctcaccacgctgagcgtcatcgggtccaccgcgccggtactcgctcccgtggccggcggcctcgtcctcggtgtcggctcgtggcggctggtgttcgtggcgctggccgtgaccgggctgctgttgctgaccgcgatctgggcgtgggtgcccgaatcgctgcccgccgagcggcgccggaccggcggcctgccggccgtgctccgcgcgatgggcggactgctccggcgccgggagatggccggccacctcctggtcatgggatgcgggctggccgccctgttcgcctacatcaccggctcgcccttcgtcttcgagaacggctacggcctgtcgcccacccagtacagcctcgtcttcgccaccaacgccgtcggcacggtgctggccgggacggtcttcgggcggctggccgggcgggtacgtctcaacctcctgctcaccgtcggcgtggccgtcaccgtggtcgccctggtcacgctggtgaccctgctcgccgtcggcatcagcacgttcctcaccacgtgggcctgcctgttcggcctgacctccgggttcgggctgctgctgcccgcctcgaccaccatcgtcctcgcggtcggcggcgacgcgcccggagccgcttccgggatgctcggcggcgcccagttcgtcctcggggccgcggccgcgcccctgcccggcgtactgggggacaccaccgcgatgtcgacggcggtcgtcgtcctgggctgcgtactgctgtcggcgacagccctgctcacgctcgcccggccgtgggagggccatggcgagccggccggctcacccgagtga

SEQ ID NO: 33 - DNA, 561 bp, Streptomyces sp.
  source 1..561: mol_type = genomic DNA
atgcaccggtccatcgaccttcaccacaccgccgacacgttcgagaaatgtgcgcagttgtgccgctcggcccgggaggcgaccgaccgggccgtccgcatgcgggccgaggcggcggaggccagactccgggccgtacggatgcgccggaccatggagcggatgaaagccgggcaccgttccgacgacgcccccggatgccggcggcttccggccacggaaacccgccgcggcgccaccgcccgagcggcgcagctcgcgctgctgaccgctttcgtgcacgcccgcctggacgaggagggcggcaccgcggacctgttccacgagaccggctgcccgggcgccggggcgtcggagtgccactgtcccgtcccggagcgggtacgccacgacgtcgccgtccggcacggcctcgcccgggactgcgaggaggcgctgcacgaccccgatcacgacgcgtgcgactggccgcacagcgaactggccgccctgctgcggttgaaggtgctcgcgctgccgtacgaactgcaccggcagtggcgggaggagtggagaccgtga

SEQ ID NO: 34 - DNA, 759 bp, Streptomyces sp.
  source 1..759: mol_type = genomic DNA
atgaaccagaaacgggagatcgcggcgctgatcggcgatgtcttcggccccgcggccgtcggcagctacctgtacggctccgccgtgctcggcggcctccggccggccagcgacctggacgtcctcgtcgtctcccggcggcgcgcgaccgtaccgcaacggcgcgccctgctcgacgggctgctcggcatctccggcttcacgggaggggcccgcccggtcgaactcaccgtcgtcgtccaggaccaggtacacccgtggcggtacccgccgatctgcgacttcctctacggcgagtggctgcgcgacacgtacgccgccggtgtggtccccgagccggaaccgctgccggacctggcgctgctcgtcaccatggcgctggccggtgaccatcccctcacgggcccgcccccggcacaggttctcgaccccgtcccgtgggcggacgtggcccgggcgagcgtgcacggcatccccggcctgctggacgaggtggacagcgacacccgcaacgtcgtcctcaccctcgcgcgcgtctggaccaccctcgccaccggcgagatcaggacgaaggacgccgccgccgactgggccctcggccacctcccgccccggcaccgcccggtcctgcgccacgccagggagctgtacctcacccgccggtactccgaggagagctggagcgacgacctcagatcgcgggtgcgcccgcacgtggaccacgtgctcgccgagatccgcgaactgccctacgcctga

SEQ ID NO: 35 - DNA, 1008 bp, Streptomyces sp.
  source 1..1008: mol_type = genomic DNA
atgacggaagcgggccggagcgttccgccccggcggcgcttaccgttccggcagttcgtgctcaaggtgcacagcaggtgcaatctcgcctgtacctactgctacgtgtatgaggggcccgaccagagctggcgcgagcgccccgcccgggtggccgaggagaccgtacgccgcaccgcggaacggatcgcccggcacgtcaccacgcacgggctgcgttcgatacgggtcgatctgcacggcggggagccccttctcctcggccccgccgcgctgatcgaccacgcgcgggccatccggaacgcggtgcccggagactgcgccgtcgccgtctcggtgcagaccaacggcacgctgctcaccgaggacgccgtgcggcggtgcagcgcggccggcatcagcgtcggtctgagcctcgacggcggcagtgcccggctcaaccaccggcgtgtcgaccgcgcgggccgctcctcctggcccgcggtacggcgggcggccggtctgctggccggtgtgcctgacgcgtacgggggcatcctgtgcaccgtcgacgtggaggccgaccccgccgaggtctaccggtcgctgatcgcgctggcgcccaccggcctcgacttcctgctcccgtacgggaactggagcgcgccgccgcccggcaccggcccgttcgccgggcccgtcggccgggcagcgcgtcccgtcccgtacggtgcgtggctcgcccgggtcttcgacctgtggtgggacacgccgccgggccgacgggccgtaccggtccgcctgttcggcgagatcgtggcgctgctgctcggggcggcgagccggtccgaggcggtggggctgtccccgctggccgtggtggtgatcgacaccgaccgttacctgcacggctccggcttcggtcacccttcggtgtactgcgcggatctggagcatctgatccgccacatcggcgagcggcttcaggaagaggtacgaggggtgccgggcgatccggtacgtccgtga

SEQ ID NO: 36 - DNA, 663 bp, Streptomyces sp.
  source 1..663: mol_type = genomic DNA
atgaacgcagaggcggaagcgcaggcgcaggcagcggcagcggcgcggactgagtcaccagtacaggcgcagacagcggcagacctcatcgggagcgagcccgcggcgaccggctggccggacgtaccgggcgacaccgccctcacgatcaaggtcccggaagccgacccgctggtccgcgcgggcttccccgcccacgtgaccgtgctctacccgttcctgcacgagagccgcatcgacgccacggcgcagcacgacctcacccgcctcttcgccgggcacgacgccttcaccctgaccctcgactcgttcggccgctaccccggcgtgctctacctcgacccgtggccccacgcccccgtgaccgcgctgaccaaggacctcacccggcgctggccggaagccgtgccctaccgcgggatattcggcgccggactcgccccgcacctgacggtcgcgaaccacgagggccccgcgacccaggaggccgcgtacgacgcgttgcaggccgagctggctccgtcgctgccgctgacctgttccgtacgggaggtccacctcatcgtctgggacgggcggcgctggcgggaccggacggcataccgtctgggaccccccgcaccgtgtcgcttgaccttgtcgcagcgacaacgtttctactga

SEQ ID NO: 37 - DNA, 798 bp, Streptomyces sp.
  source 1..798: mol_type = genomic DNA
atgcgcatcggagaactcgccggcatcgtcggcgtcaccacccgtgccgtacggcactaccaccgcaccgggctgctgcccgagccgccgcggcagcccaacggctaccgcgagtacgcgctgcgggacgccatcgagctggcccggatccggcggctggtcgagctcggcctgagcctggacgaagtcaaggacgtactggcggacgacgcgggcaaggacctcgtcgagatcctcaccgagctggacgccgacctggcacgccaggaggaggccatccggcagcgccgcgcccgcctggcccggctgttcgaacaggccgagcggaccggccggctgccggaggaggcccccgtctcgcccgagctcgcggccgtcttcgacgacatggcccgcgcctccgcgcaggtacccgggccggagccggccatggcggccaaggaacggcagctcctggctctgctggagaccggctcgccgggcggcgaccgcggctggttcgacggtctggtgcacgccttccgggccgacccggcggcgatgcggcgcgcgtacgacgtctatgcccggatggacgagctggccgaggccgccgaggacgacccccgggtggagctgacggcccgggccatcgtcgacagcatcccggacgaggcgctgcgggcgatgcgtctgccggccgacggcgccgaggaagcgggcgacgggttctccgacgccttcttctccgacttcgcacccgcccaggccgccgccgtgtaccgcgccatcgaactgctcacggagcgtgccgggtga

SEQ ID NO: 38 - DNA, 924 bp, Streptomyces sp.
  source 1..924: mol_type = genomic DNA
gtgaacacgctgaggaaggccgtgcgctggtccttcgccgcactcgtgcccggcgagctgatactcgtcctgtgtgtggccggcggagtgcgcatcgccccggccgtccggctcgccacggagccggccatgctcacgctcgcggccgcggcggcggtgctgatcgctctcgaccaccgccgccaccgccgcgccgggctcggccaccgcgccgccttcgccgccgcgatcgccgacaccgtcccggcgccggtgcgcaagctcaccgcccacgaggtgttcctctccaccagcttcctgcgctgggtgacccgccgcggcccgcacggggtgcgcgacggcgacctgtccgtcccgtacgcgccgggacagacggcgaccatgtacggcttcctgttcgtctgcgtcgtcgagaccgtggggttcgccttcctgatcccgtggccggtggtgcacgccgtgacgctcgtcctcgacatctggggctgctacttcgtcatcgccctgcacgcctcgtgcgtggtgcgcccgcacgtggtcggcgccgacggctcgctgcgcctgcgtcacggagccctgctggacatccgtatacccgccggccgggtggccgccgtccgccgggaacagaagttcccggacggcaagctggcagtcgtggacaagaacggtgtcgccgacatggccgtggccggccggacgacggtcctcgtcgaactcaccgaaccggtgcgcttcgtacgcgccctgggcaagaccgccgaggcccgcgccttccgcttctacgcggaggaccccgcggccgtcgtcacggcgctgaaggccggtgccgcgtctccctgcttggcctcgacccgcccgagcgcgtccgggccggacgccagcgcctcgcacgccgcctcccaggccgggtccccggggtag

SEQ ID NO: 39 - DNA, 1017 bp, Streptomyces sp.
  source 1..1017: mol_type = genomic DNA
atgcatgcatcgaattccccggccggcaccctcggctcacccacccccgtcctctcggtcggcccggtggtgctgcccgtgcccggccgggccgtggacctccagctgcggatctccgcgccggtcaccgggagcgacctgccggtcgtcctcctctcgcacggccagggttactcccaccacctctcctcgctgaacggctacgcgcccctcgccaactactgggcggcgcacggcttcgccgtcatccagcccacccacctcagttcccggaccctcgacctggaccccgccaccccgggcgcgcccctgcactggcgctcgcgggccgaggacatgcggcacatcctcgaccggctcgacctgatcgaagcggcggtcccgcagcttccggggcgcctggaccgcgagcggatcgccgtggccgggcactcgatgggcgggcacaccgcgagcctgctgctcggcgcccggctcaccgacccggacgacggaacggtggtggacctggccgagccccggatcaaggcgggtgtcctgctcgccgcgcccggcaggggcggtgacgccctcaccgcgtccgtggccgaggcgttcccgttcttcgcgaccacggacttctcccggatgacgacgcccacgctcgtggtcgccggggaccaggacgacaccgcccacctgacggtccggggtcccgcctggcacgccgacccgtacgtcctctcccccggccccaagtccctgctcaccctgttcggcgcggagcacgggctcggcggggtctccgggtacgacgtcgccgagaccaccgacgagagccccgagcgggtggccgcggtgcagcggctcacctgggcctacctccggtcgcggctctaccccggggacccggcctgggaggcggcgtgcgaggcgctggcgtccggcccggacgcgctcgggcgggtcgaggccaagcagggagacgcggcaccggccttcagcgccgtgacgacggccgcggggtcctccgcgtag

SEQ ID NO: 40 - DNA, 192 bp, Streptomyces sp.
  source 1..192: mol_type = genomic DNA
atgaacaccggcaacacgacggacaccccggcagccgcccggcaggccaagttcggcaagctgcccgagcgcatccgattcgaggacatggtcacggagcggccctcaccggagagccgcacgccgaacgcgtacacccccgaaggcacgtggaaccacttctcgtgcctggccctggatctcggcctgtag

SEQ ID NO: 41 - DNA, 564 bp, Streptomyces sp.
  source 1..564: mol_type = genomic DNA
gtgaacaccgaccatgtcttcgtctgcgggcaccccgccctcgacttcgccgcgacgctccgcgcccggcgcaccgtccgcttcgagatgttcgcgacgccggagcggctggaggcgtggtacgtggagtccgggctggtggattccgtgacgcccgcccggccgtccgacgtggagcgcgccaagtccgtacgggaggcggcctacgagctgatcaccgcgcgtcgcctcggggagccgttcggcgcccgggccttggacgtactcaacaaggccgcacgaaagccctcggccgcgccgcaactgacgagcgccggccgccgcaccgacgccaccccggaggaagcgctgtccgccgttgcccgccaggcggtggagttgctgagtgggccggacgtgccgctgctcaaggagtgcgggaatcccgagtgcacgcgggtctacatcgaccgttcgcggggcgcgcggcggcagtggtgcggtatggagtcctgcggcaacaagatcaaggccgccgcctaccgtgcgcgcaagaggggcgcacccgccacgaccgcgcgctga

SEQ ID NO: 42 - DNA, 1758 bp, Streptomyces sp.
  source 1..1758: mol_type = genomic DNA
atgccccgacccgccctgctcgcccatgatctcgtccgtacgctggggacccgacgggtcctggacggcgtctcgctcaccgtctcccccggccggcgcgtcggtatcgtcggcgagaacggggtcggcaaatcgaccctgctgcgcctgctcgcgggcgtggacgaacccgactccggccaggtcacccgcccggacgacctgggcttcctgcaccaggagatgccgttcgacggtgccacgacgatcgcgcgggtgctggccgacgccctgcgggaggcccgcgccgccctggccgaactcgaccggctgacccggctgctcgccggcaccccggacgaccaccccggccacgccgaactgctggacgcgtacgggaagcagctcgaacacgcccaggagagcgaagcgtgggacgccgaccggcgggccgcgatcgtcctggacgggctcggcctcggcgggttcggacacggccggacgctcggttcgctgtccggcgggcagcgcggccggctggcgctggccgcgctcctcgtgcgccggcccgccgccctcctgctggacgagccgaccaaccacctcgacgacgaagcggccgccttcgtggaggagcaggtccgcgtacagcccggcgtcgtcgtggcggccagccacgaccgggcgttcctcgacgcggtctgcaccgacctgatcgatctcgacccggcggtgcacggtccggtccgcttcggtggcaactacagcgcctaccgtgccgagaaacaggcggaacgcgagcgctgggaacagcggtacgcggcggagcaggaggagttggaagcgctgcgccgctcggccggggtgaccgcgcgccgcgtcgccccggaccgcgggccccgggacaacgagaagatggggtacggccaccgggccggccgggtgcagcaacaggtctcccggcgggtacgcaacgccgcccgccggctggacgaactggaacgcacccaagtcggcagaccgccgaagccgctgcgcctgcacgccggggcgctgacgggcgcgctggtggacggtgctccggcggacagcggtcaggtgcacggcgctacggcggacagcgctcccgtggacagcccgctcgtcgcgctgtgtgacgcgcgggtgcccggtcgcctggacgtcgcgcgcctggacatccccgccggggaccggcttctggtcacgggagacaacggggccgggaagtcgaccctgctgtccctgctggccgggcggctcgacgtcgagcgcgagggcgcggtacgcagacggcccggcctgaccgtgggcctgctggcgcaggacaccgtgttcgagcacccggaccgctcggcccgcgacacgtacacccatgtgctgggtgccgagcgggcggaggcggtgccgctcggctcgttgggactgctgggcgtcacggatctcgaccagccggtggggcggttgtcggccggccagcgccgcaggctcgcgctggccctcctggtggcgcacccgccgcacctcctgctgctcgacgaaccgacgaaccacctctccccgcgcctgtgcgacgaactggaggacgccctcggcgccgggccgggcgcgatcgtcgtggccagccacgaccggtggctgcgcgggcgctggaccggccgccggctgcggctgcgcgcgggccgcctggcgcctccggacgacgtgtacggcatacgcagtacgggaggcaggctccggcccggcacctccgaccggtga

SEQ ID NO: 43 - DNA, 1635 bp, Streptomyces sp.
  source 1..1635: mol_type = genomic DNA
atgtccgacgctttcgtcgtctgctccagcctgtccttttcctggcccgacgacacccccgtcttccaggacctgtccttcaccgtgggcaccggccgtacgggtctggtggcgcccaacggctccggcaagagcaccctgctcaagctgatcgccggtgaactgcgccccgccgccgggaccgtcaccgtctccggcaccctcggtcacctcccgcagagcctcccgctcgccgccggcctcaccgtggccgaggtgctgggcgtcgccgaggtcatccgcgccctggacgccgtggagtccggcgacgtggacgagaagcacttcgcgaccatcggcgacgactgggacatcgaggagcgcacccgcgcccagctcgaccggctcggcctcaccgaactgaccctggaccggggcctgagcaccctcagcggcggtcagatcgtctccctcggcctggccgcgcagctgctcaagcggcccgacgtgctgctgctcgacgagccgacgaacaacctggacctcgacgcgcggcacaagctctacgacgtactggaggactggaacggctgtctgctcctggtgagtcacgaccgggccctgctcgaccgcatggaccgcatcgccgagctggagccgggtgccctgcggctgtacggcggcaacttcaccgcgtacgaggaagccgtacaggccgaacaggaggtcgccgagaagaacgtccgcaacgccgagcaggaactcaagcgggagaagcgggagttgcagcaggcccgcgagcgcgccgaccgccgggcgagcaacgcctcacgcaacctcaagaacgcggggctgccccgtatcttcgccgggaacatgaagcgcggggcccaggagtcggcgggccgggccggccagatgcacgcctcgcgggtcagcgaggccaaggcccggctggacgaggcgggccgcgccgtacgcgacgagcagcgcatcacgctggacctgcccgacaccgacgtgcccgcggggcgcaacctgttcctcggcgagggcctgcgcgtcagccacggcgagcggccgctgttcgccgacggcggggtcgacctgacgatcagggggcccgagcgcatcgcgctgaccggccccaacggcgccggcaagaccacgctgctacgcctgttgcagggcggtctcgaacccgacgcggggcagatcaagcgggccgacagccggatcgcgtacctctcgcagcgcctcgacgtgctcgatctgtcccggaccgtggccgagaacttcgccgcgttcgcaccccagcgtacggcggcggaacggatgaacctgctcgcgcgcttcctcttccggggctcccgggcgcacctccccgtccaggtgctgtccggcggcgagcggctgcgcgccaccctggcctgtgtgctgtgcgccgaaccggcgccgcagctgctgctcctggacgagccgacgaacaacctcgacctggtcagcgccggccagctggagagcgcgctggcgtcctaccagggcgcgttcgtcgtggtcagccatgacgaacggttcctcgcccggatcgggatcgaccggtggctgcggctggccgacggggaactgcgggagacggcggtccccgaggtgtga

SEQ ID NO: 44 - DNA, 963 bp, Streptomyces sp.
  source 1..963: mol_type = genomic DNA
atgcacgcgatccgtatggaccggcacggtggccccgaggccatgaactggaccgccttaccggacccggcccccgggaagggcgaggtgctcgtgcggctcgccgtcgcgggagtgaacttcatggacgtgggggcccggacgtccggcggccccggctgggccgctccggcgattctcggggacgagggcgcgggccgggtgacagccctcggggagggcgttgagggcttcgccccaggtgaccgcgtcgcctggttctaccaccccggcagctatgccgaactgctcgccgttcccccggaatcgctggtcaagctgcccgacgaggtgtccgacgagaccgcggccgcggtcatgatgcagggcctgacggccagtcacctcaccaccgagacctaccccgtagggccgggggacaccgccgtcgtgcacgccgcggcgggcggcgtcggactactgctgacccagatgatcaaagcgcgcggcggccgggtgatcgggctggtgtcccgggaggagaaggtggcggcggcgcgggaagccggcgcggaccatgtcctggtctcccgtggagggggcttcgaagaccgggtcagggagctgacggacggcatgggtgcgcaggtggtgtacgacgggggcggcagtgcgacgttccgctcctcgcaacgggcgctgcgcccgcacggagttcacgcctactacgggccgttcatgggcgtcccgtccgtcgctccgaccgacctccccgacagcatttttctgacgtaccccgtggtgcaccaccatgtgcccacacgtgcggccctggtccagcgcacgggagaggtcttcgacctgatccgggacgggcggctgaccccgcgcatcggggggcgttacgccctgtcggacgccgcgcgggcgcatgccgacctggaatcccggcggaccatgggcaagctcctgctcgtaccctga

SEQ ID NO: 45 - DNA, 600 bp, Streptomyces sp.
  source 1..600: mol_type = genomic DNA
atgccgctgacccgtaagggcgccgccacccggcagcgcatcgtcgagggcgcggcctccgagatgcgccggctgggcatcttcgccctgacgctggacgacgtcatgcggcgcaccgcgaccagcaagagtcagctgttccactacttccccggcgggaaggacgagctgttgccggccgtggcccgccacgaggcgtccggcgtgatcagcgaccagcagccggaactggcctcgctgaactcctggagcgcctggcggagttggcgggacaagatcgtcgagcgctaccgcgagcagggtcccaactgtccgctcaacgtggtgatggggcagctcggcccgacgacggagggggcccaggccgtggtgcgggaactgtacgcccggtgggagggcgaactggccgccggcatccgcggtatgcagagccagggcgagatcgacgccgccctggacgccgggcgcggcgcacgggccctcctggccggcattcaggggggcgtgctgatccacctcgcgaccgggagcctggagtacctaaaagcggccctggaccaggggatcgagttcctccgccggccggcggcgagggcgtag

SEQ ID NO: 46 - DNA, 369 bp, Streptomyces sp.
  source 1..369: mol_type = genomic DNA
atggatggactgaccgagtcgaacaacggggtgcccgaaccgcacagcggctggacatttgtgaccaatcacgcccgggtgctggccgctatcgccgacgaccgccacgcccgtatccgtgacatcgcggcacactgccggttgaccgagcgcgccgttcagaagatcatctcggatctggagcgcgacggttacctctcgcacacccgcgagggccgcagcaacacctaccggatcgaccccggcaaggtgctgcgccacccggccgaagcgggcctgaccgtggcgtccctgctgtccctgctcgtccaggacgagcaggaccgcagcacgcaaccggacggccgccggcaccccagtacggcctga

SEQ ID NO: 47 - protein, 226 aa, Streptomyces sp.
MREQKIMSRLSASNSVMYELASQNPNMSDLDRLTNLYWALCSVLRAEVPGEVVELGCNAGLTSVMLEMVLEAESPDRTLHLYDSFDGLPAPSHADQYLREGDCKATEADVCAAFERWGLRRPEIHAGWFDTTLPQQLPDRVAFAYLDGDFYESILTSLTHVWPRLSVGGSVLVDDYCDRLASPRAWDGLPGVKKACDAFFADVPVRSRVLVGAGDLAFAEFRKTEA

SEQ ID NO: 48 - protein, 222 aa, Streptomyces sp.
VLHDSLKIGSVEGLLSPDEVTRLTACVTDGLGADRRFTVSERRRSVHSLEGESLARVKEVYEPHGRVEYDELPDEITGVLEGAVARRLGELRRIFPTVRRSMDWLYVEYGAGQYVTPHVDYPFNEIAPDRPKVAAIGIVLHKAEAGGEFFVETGGGDALWNDEGEVKEGANDHSPWFNAMRRTRWHVTSRAGDAVCWGTQLVHGTEPVAAGRSGKFIGFLGS

SEQ ID NO: 49 - protein, 296 aa, Streptomyces sp.
VKTDFPYHPSQDRRGDRLTPPVAERNARRSRGVAGRIVWAHSLWIDPELERLYGGDRHAPDVNRERAVRLSDGRLDPLAMRPLDSLVSVTGPFLSRHTSTVGVDPADEAFDLVRRTHSGYLCELAIGVIRPGSPQRGVTPSGPDHRERSPFTAVVPRDHDAVSRAAGLLESSANELLSQWYEAFTHARGTTHGILLPPLRWAQGVFALSIRYEYCRRVRDEPGFTAGERQHAGLLLDEVTEALPQPGAFRDSLPGGVQGSVVNELLSCSYPLWDRVRFCLDSPFDGLRSLAEHSTA

SEQ ID NO: 50 - protein, 239 aa, Streptomyces sp.
MSHAAFPAVESATHNAEGEHGHHRARFPYVALEGLNGAGKSTLRNSLEESFWALGMNVYTIGQHSWQSPGAARVILDIKERRHTHPPERIQLAYLDDRRAQGDNILSRVLHEVPVIGDRSLVSDIVYLEALDGIPAEDTFALYRRHAVILPDLIVHVDVSAATALANLGKRERRRKPHENPEQMRSLAATYESVLWAQRLPGLPPVLRYQPEVAADGTSYTNPAASIGDRILASLGLAS

SEQ ID NO: 51 - protein, 194 aa, Streptomyces sp.
MSAPGRAIVLAGTSGAGKSSTAEELQKTLPDAFLVLGLDTFFGMVPYRWTSAGTASAEGFSYIRDTDPHDGAARLRIGYGPVGAHLLAGMRRATEALLREGNNVIVDEMPIDSGVVPAWKRDLLGHSVLWVHLTAPLPVLEAREAERFPEKFRGLSRGHFDVCVPADFDIVVNSAELDPRERADRIAAALPPRR

SEQ ID NO: 52 - protein, 408 aa, Streptomyces sp.
MSDARSATPAVPLRRNRDYNLWWSGAAASQTGSFMVTVAVPMLILTNSGSAGQAGLVTGASAVAKFVVAFPAGAVADRCSRRRLLLLSSLMQLAAVASILIAVASQHVRLVHLAAAALVEGAGYALHSATELPLLRKVVPEPQRRAALGREQGRKAAAQLTGPPLGGTLFSWVPWAPFLAASVSFLGVTTAALGIRAPLGPQRRRKEAAPPFFPEMLEGVRYIRRSAFMSYMLLWFALVNGAFAGLSFLLVVLCHDRGASASAVGTAQGVGSAGAVLGALASAWLTARLTHRQLLKLASWVLVIGSAAMAVPTLSPLPTGLAYGVSLFLTPAVNVSFTHHMVGTVPDALTGRVSMTMMTAARSLNWLFVIAVGLAAERWDPLVPLFTLSSLFLALACANHLRRVRTQR

SEQ ID NO: 53 - protein, 42 aa, Streptomyces sp.
MAVRPVIKARNTHLICENAARGGVRDARGVTVVPALPLRAQR

SEQ ID NO: 54 - protein, 1125 aa, Streptomyces sp.
LTTSLWSRRRLLSTLAGAAAVPALPSILTLNAGVAHAAGPADGNGPGDVLPTTERGQAVWAYKTGGRAVREAAAAALVGSPAAVTAFLTRELPAARAEDNRFAIIMGLSQGGKAVNETAGAALTAGDEAVAAYLRDGYKKPVEEDLRVAVFTVMNNSGVAVRREADKALRENTRSAFQKFLGETQYTAQQEDERVEVFSILNNASPEVKKYAQRALSEGTPEAIRQFLSSGQHIARARDEERATIDQLVEIVQRESRQAKLQTENAVTASERAKSAAEKAKAAALLAAEHAKAAEQDVRKSADAATSAARAASGAADAAGAAIAASRTAQSASHRAAQAAQAAAAAAAMAGHAASRAYNAAIAASKDASKASAARVAAQGARNAAAQARTAAKAADQATIASQQAAVAGTAAAGAARSAASAANAAAESAAAAGAAQSEAAKARQAAAEADAAAARATRAAATARSLANQAAEAAGVARDAANSAADHAEKAAAAAEEAAKNAGKAVEYAKRSAAYAEAAMEAATKADNAVKKALQVEKAAREAEAARIAADTELGVLEARLRAKAETEDAKRAERQRAQADRATQRVKDLIAAAEAALRAGDTAKAVASGRQAAVGLLESSGTWTREAAEFALAGTDQDTVNWIDTDRLLAQQQDDRETVLTIATSSTADVAEAAHKVLAGDSPEAVRDFLDRGVLEAAATDNRVALFRVLDENPGRAVREKAEAALKDGSAQALHRFLTEELTEAVKEDDSVEVFRLLGSGGPYMQSAAKIVLEGSARMRRGFVSHDQYNIARLDHDRATHVAAIQSSIALAAKVAAEALESAARASKAAAEARDAAADATDWANKADAYAQRATDSATEARRNADAADKSAAAAAQSAKEASNAAVTARGAARAANFSMRQASASAQQAVASAWQAQASASRAQASATQAGKAAKAAAAAASEARRIVFVKRKAEAEAAAKRAAEKAKEHQSNGTNPSQTSDNDKPWYVENGVWPEDVKSAKDWAQVTGHWSTIAGGAAVALGVGGFFFAPLGGAALAVGLVSWGLQGASALLNYIGNGPDSSEFHSALGMFAVGGILLGTGQLLSKFGKMPAFGPAVEQVGEKIANVAHDITTTVAGWLTW

SEQ ID NO: 55 - protein, 125 aa, Streptomyces sp.
MLTSTLSETQWWVAAVLSGFALAVAAQSMATTARDKLIAASLPLAGAIAIGVSSAARGYTDTLPLLLYTTTMIVLVILRLLFAKYINRQLALKRSGKPMENMSGKQTAIFLLAFMATLVLVSFVI

SEQ ID NO: 56 - protein, 167 aa, Streptomyces sp.
MRITRRLIVTGTVLAASAATAVTVAQATGNPAPATVTVAGAEPGSAVEDFSYPNADKIKKELGIDLKRGDGHIMLVDCNNDGRLMTIWSRTIGQVCFRVTGDTGYLSLSIPKVYGVKADADHKANLTLTADGSKQNIALGKNESGSAGETADPERRPHDLVEISTSK

SEQ ID NO: 57 - protein, 49 aa, Streptomyces sp.
MDVQMSTEADSDLGELEVLEMDELQVSGSSTAYNYIAKNADRSSGIPAG

SEQ ID NO: 58 - protein, 279 aa, Streptomyces sp.
MICCPPRQMDVPGQSRRIHRAISRAGANWLKKATDMPTARGLDASSASASAARRFRVAKSREPACGSDNRSTAWSSASKSSHTCDSADSRSISWVACRARRTARTGPRRGAGFFVRKVNSYLNDTVVLSMVSCVGCYWGRCIFCSYGNRSLERGAYQQASPGQLADTVCRIVKSTGIDFVTVVDENTNLLLLVRAMRLVRERGLRVRFNTRNRLEPILLDSAFCQELAELGCEGMAVGYEGVTQRLLFTVLDLRIADFGRQVGVTDWPARYAVLRRQED

SEQ ID NO: 59 - protein, 257 aa, Streptomyces sp.
MDDTTLISRFLRDGFVKLEGAVAPRVAADCARLLWRETGCDPDDPTTWTQPVHWVPGLAQGPFAAAPNSPALHHAYDLLVGAGRWEPRYSLGTFPLRFPHEQEPDDAGWHIEGSYLPEGESWYFTNLRSRGRALLMLFLFSEVGDQDAPTRIRVGSHLDVPKVLEGYGEDGASGLTLAPDLVAASDHRPLTLATGSPGDVFLCHPFLVHAAQPHHGRRPRFMAQPPLMPAAPYELERADGAYSPVEIAIRRGLGRDR

SEQ ID NO: 60 - protein, 59 aa, Streptomyces sp.
MARRQPHAPERLLVAAHPELREREALKELGLIASMTDALKRRGVPDLTARVAAELGALA

SEQ ID NO: 61 - protein, 314 aa, Streptomyces sp.
MSRAVVYESFGGPEVLELREVPEPHAGPGEVRVRVTAAGLNPMDWGIASQPEAAARFGIAVPAGFGFDFAGAVDEVGAGAEGFAVGDRVHGGALGRAVADFVVVKTPTGTTDPLWHTPEGISDEVAATLPVAGMTAAAALAAIDLRSGDTVLVGGAAGGVGVFVVQLAQLAGARVIGTAGPGTFPFLRRFGVEPVTYGPGLADRVRTLAPEGVTAATDLFGIETAETALALGVPAERISTIAAGPNPPGGARATGGFDASPDAMERIGDAILAGAITVPIAAAFPVEQIREAVTLQAGRHVHGKVVITLRPGPH

SEQ ID NO: 62 - protein, 66 aa, Streptomyces sp.
VQTLAPLLAEGSGVVLTASAVNVLGLPMLSAYAAGKAALRSMTRSLARELLPRNRGRRWHSALTPV

SEQ ID NO: 63 - protein, 288 aa, Streptomyces sp.
MHTAAVSDVPVGNRTELGDFLRSRRGRVAPQEAGLPPSAGRRRTAGLRREEVAILAGVSIEYYRRLEQGKQRRPSPAVLEAIGQVLKLTDDERRHLAELSRGRPPSPVTGARTGRASRAVREEVRRMLEIAHPYPACVLSRSSDLLTANQGALRLFPGIEDWPEPERNTARYAFVHPRARELYADWDDVARGVVAHLRAAVVVHRGAPDIREVVDELSARSTAFASLWQRHDVRARTNGRKVYHHPDAGRLTLTYEAYDVARSEGQRLIVYQAPPGTPDHEALLRLSA

SEQ ID NO: 64 - protein, 494 aa, Streptomyces sp.
MGGAEAHEEGTVGPAVSTQAEPTRRGRQLVLAVCCTSVFLSSLNNTVLNVALPAMQRDLRSPLSGLQWTTDAYLIVLAALLMLAGSAGDRLGRRRVFRTGLALFTLGSLLCAFAPGLGWLIAFRTVQAVGASMLNPVAMSIITNTFTAPRERARAIGVWGAVMGVAMAVGPLVGGVLVQGLGWRSIFWLNLPVGAAAFVCAARFVPESRAPRPRRVDPVGQALVVVLLGCLTYAIIEGPGTGWTAPRTLACALAAPAALLALVLHERRRTEPLVDLRVFRGAGFSGASAMAVLSFFVLGGFLFLNTLYLQNVRGLSALAAGLCLLPMAVMNIVTSPLSGRLTATRGPRLPLLLAGAATAAGGVLLATGGALTTDALLFTAYVLIGVGAGLANTPVTDAAMSGMPREQAGVAAAVVATSRQFGQALGVAVLGAVLTAGLHRSGTPFTRAVLPGYWIVTACGLAIVVLALATTRRVVPAASPAGRRRRQAAGARLG

SEQ ID NO: 65 - protein, 204 aa, Streptomyces sp.
MPKTVDRDEQRRQIGAALLSLASERGLDEVSVRTVATYCGRSPGAVQKYFRSKDEMLAFAAEMAGERIEARMAAVDPGLPPRAALRALVLTTLPLDAERRAEAAAQLAFAVRAAHHEPLAEVRRRVDQDIREALAHWLTSAGYGHGPDPATDCLAIADAVIALSDGLALRLLYTPQAHDDLVRALDRGLEALVPAQAPGEPDAR

SEQ ID NO: 66 - protein, 92 aa, Streptomyces sp.
MDDRLDPVTRASDQDREKALARERDRKDLREWLAEWRCWLGGGVILSAVWGVRCLRKGELGFYWPVTPLGIWAAVLVAIAVWPRSGGGGGEG

SEQ ID NO: 67 - protein, 7698 aa, Streptomyces sp.
MGRVVPGTPHHSRPLSSAQRGIWFAQQLDPDSPSYHTAEYVEIEGPLDLPAFETAVRRAVTETDSLNVRFTDDGQGPCQTIQRGGTWSYRVFDVGAEPDPRAAAEAWMRAETDRPGGLEAGRTVTLALLRAGADRAFFYLRAHHLVTDGYMCALFTHRVAELYTALVTGAPQREDSSGSLDLLLADDAAYRESERFAQDRAHWLDRLAGRPEPVNLAGRTARVSRSFLRYGTRVPDATADGLRALARRAGVGLPVPVIAATALYLGRMTGADEVALGIPVTARSGGGRHIRGPMSNELPLWVEVRPGMSGRDLLRQVSAGARQLLRHQRYRYEDIVRDVRTVGGERRLFGPTVNIMSFATELTFGEARATAHNLSVGPVTDLSFRLDDRLGGGALHLDVDANPALYDAAETAAHQRRFLRVLESFAAADPERPVGRIDFLLPGERAEVLPVRPAATDAPEPDEPGPTLPTLFEAQAARTPDAVAVVTEDGETEDGELTYAGLNHRANRLARLLLKRGAGPERLVAVALPRSADLVVALLAVVKTGAAYLPVDPAHPAERIALMLRDADPVCLVTTGEPAAGAPYGADVVRLDDDATVRALADGPGDDLTDADRAGQLDPAHPAYVIYTSGSTGTPKGVAVPHRNVVRLFRQTEAWFGFGPDDVWTLFHSYAFDFSVWELWGALLHGGRLVVVPQDVSRSPERFLDLLVRHRVTVLNQTPSAFYPLIRADHDHPELSARLALRTVVFGGEALDPGRLRAWYERHPADAPRLVNMYGITETTVHVTHAPLDERDAAADPPRSVIGEPIPDLRAYVLDNALRPVPPGVTGELYVAGPGLARGYLGRPGLSAQRFVADPFGRPGERMYRTGDRARRHADGRLDHAGRADDQVKLRGFRIEPGEVEAVLARHGAVAQAVVLLREDRPGDRRLVAYAVPAGGPRPESAALREFAGRHLPGHMVPAAVVVLDRLPLTANGKLDRAALPSPEYTPGAAHRPARDAAERVLCDLFAEILGVRRVGVDDGFFDLGGDSLLATRLLSRIRTALGAELPIRVLFDTPTVAGLATAVRESGGEARPVVRRTERPEALPLSFAQRRLWFLNRLEGREAGADYNMPFVLRLEGALDRAALEAALTDVVTRHESLRTVFPEVDGVPRQVVRDPGAATVTLPARDVDAAELAEVLARSAATGFDLAAELPLRAALFALAPDRHVLSLVVHHIAGDAWSMVPLGRDLSTAYAARLRGEEPSWAPLPVTYADYTLWQRDLLGEERDEDSLIARQVAYWARTLAGLPDELALPADRPRPGVASHRGGTVPLRLDAALHVRLAALAEAHGVTLFMVLQAGLAALLTRHGAGTDIPLGSPVAGRTDDALDDLIGCFVNTLVLRTDTSGDPGFGELLARVRETDLAAYAHQDVPFEHLVEVLNPTRSLSRHPLFQVLLTLTGRTAAPDALPGLRAEAERVESGTAKFDLTFNLTEAVDAGSGPGGVEGYVGYSADLFERGTVEALAARFVRLLEAAAADPDLPLGALDILGEAERHRLLTEWSGTGGRTGTGAAAGGARSTASLPELFERQAAATPEARAIAAPDAELSYAQADARANRLARLLISRGIGPEQVVAVALPGSAELVVALLAVLKTGAAYLPLDLAQPGARIAGLLDDATPALLLTTSHAATGLPDDTGAGTGTDTGPARLLLDAPDTVRALERQSAARPTDADRSAPQLPDHPAYVIYTSGSTGRPKGVVVTHRGLAHYVARARRVYTGLDGTVLVPTSLAYDLAVTGLYGALTSGGCAVVAGLDDDPWLTALLARTPATFMKATPSHLVLLAALPAEFAPVRQLMLGGEALPAAHLEEWRARHPEVAVVNHYGPTEATVGCLDLHLAPGEAPGGATVPVGRPMAGMRAYVLDAGLRPVPPGVAGELYVAGPQLARGYLGRPGLTAERFVACPFAPGMGVPPAEGWGRMYRTGDLARWTADGVLVFAGRADEQVKIRGHRVEPGEVEAALAGHPSVARAAVAVREDRPGDQRLAAYVVPAGDADGVDPAELRAYLARRLPEPMVPALFTTLGALPLTRNGKVDRRALPAPALAPASTGRPPRSPEEEILCGLFAEVLGVPRAGADDGFFDLGGHSLLATRLVSRVRTVLGAELPVRAVFETPTVAGLAARLKDGSGRRRPPLRPFPRRPETVPLSFAQRRLWFLHQLEGAPNYNLPFVVRLEGPLDRAALTAALGDVVARHESLRTVFPDSGGTPRQAVLDAGPAAASVELPVRHVADEDALAATVVAAAAHRFELTRELPLRAELCALGADVHVLIVVVHHIAGDGWSNGPLARDLSEAYAARRTGQAPRWEPLPVQYADYTLWQQELLGSEDDPDSLAARQIAYWSRTLDGLPEELDLPADRLRPAVPGYTGGVVTVSVDAGLHARLEELARTSGASLFMVLQAGLAALFTRLGAGTDIPLGSAIAGRTDDALDDLVGFFLNTLVLRLDTSGDPAFEELLGRVRETALAAYAHQDVPFEHLVEVLNPARSAARHPLFQTMLTLHNNARPRLDLPGLRVSPDMAEVGTAKFDLSFGLVEERGPAGEPAGLAGILEYSTDLFDHATAEELVARFVRLLGAAADGPGRRIGELDILSPSERVRILGDWAGTSQDAPALTVPELFERQATLTPDATAVVAAGTTLTFAELRARADGLARHLAERGVGPDTLVGLCVSRSAWMVPAMLGIWKAGGACVPLDPDYPAERLAFMAADSGVRVLVAEPDLADVLEGQAAETVLLGSPDADGEPFPMPAVRADHAAFIVYTSGSTGVPKGVTVRHGGIANLFANHRERVFAPAARAAGGRLRAAHTASLSFDLCVGQLLALLAGHELHVVDEDTRRDAVALGAYVRDHGIGYLSITPTLTQELLGLGLLGAEGHQPAAILHGAEATGPGLWQRLRGLSATEVHNYYAPSECTVDAIGATVSGSERPRIGQPVAGMAAYVLDAGLSPVPPGVRGELYLAGAQLARGYLNRPVLTAERFVANPFSSVPGARMYRTGDLARWTADGALEYAGRADDQVKLRGFRIELGEVEAVLARHPDVAHAVAVVREDRPGDRRLVAYTVAARGAVPDPAGLRAHAARTLPEFMVPSAVVPLDALPLTPSGKVARRALPAPGSAVPQGGREPRTAREEILCGLFAEVLGCARPGVDEGFFDLGGHSLLATRLLSRVRAVLDVELTVRDVFEAPTVAALAARLDAGGAARPALRPVERPGTLPLSFAQRRLWFLNRLDGAGAAYAMPLALRLRGDLDRDALAAALADVVTRHEVLRTVYPETAGTPEQVVLAPERAAPGLSCRAVAETGLAAAVAAAAAHPFDLAAEPPLHAELLALDPDDHVLVLVLHHIAGDGWSNLPLTRDVTAAYAARHAGEAPSWTPLPVQYADYTLWQHRLFGDPADPDSLIARQTAYWTETLAGLPEELPLPTDRPRPAVARHRGGRVPFTLDAAAHAGLADLARACDASLFMVGQAALAALLSRLGAGDDIPIGTPVAGRTDDALDDAVGCFVNTLVLRTATGGDPAFTDLVARARETALAAYDHQDVPFEHLVEHLEPVRSLARHPLFQVMLALRNTGEAPLGLPGLAVSAVPVETTAVKFDLAFTVSERTGPQGEAAGLTGELDYASDLFDAETARRIAARFVRVLEAVAADPARKLEDLPVLDAAERERVLTGWNATERPLPPVTLPELFAAQAARTPDAVAVAGEGGTLSYAELDAWADRLARALVQRGAGPERVVAVALPRSVELVVALLAVLKSGAAYLPVDPGHPAERTRAMLDEARPVCVLDDPEGVRNPRPETAITAPPVPLSPSHPAYVLYTSGSTGTPKGVVVPHAGVVNRLLWAQAEYGLDASDAVLQKTPYGFDVSVWEFFWPLITGARLVVARPDGHRDPAYLAALIREHAVTTVHFVPSMLRAFLDEPAAARCTSLRRVLCSGEALPAQLAAHGRDILGGSKLHNLYGPTEASVDVTSWACADRPGPVPIGRPVHNTRAYVLDAALRPVPPGVTGELYLAGVQLARGYLGRPGLTAERFVACPFAPGTGVPPAEGWGRMYRTGDLARWTADGVLEYAGRADDQVKLRGFRIEPGEIETALARHGDVAQVAVVLREDRPGDQRLVAYVTSAPGAVPEPRELRESVARTLPASMVPAEIVVLDALPLTPNGKLDRRALPAPDATAQAPGRAPRTLHEEILCGIFADVLGRPLPGIDDGFFDLGGHSLLATRLVSRARAALGAELSVRDVFEAPTVAGLAARIGSGGSRRPALRPAAHRPDVVPLSFAQRRLWFLHRLEGPSPTYNMPFALRLSGPLDTAALRAALDDVVARHETLRTVFPEIKGAPGQIVLGPDETAVELRTHRTDEAGLTGALAEAAGYRFELTAEPPLRADLFALADDDHVLLLVLHHIAGDGWSNVPFARDLSTAYTARCAGHAPAWAPLPVQYADYALWQHDLLGSEHDPDSLISAQVAHWTEALAGLPEELALPADRPRPAVSAHRGGRVVFALDAELHTRLAGLAREHGATLFMVVQAGLAALLTRLGAGTDIPLGSAVAGRTDAALDDLVGFFVNTLVLRLDTSGDPAFSALLDRARETDLAAYAHQDVPFDHLVELLNPARSAARHPLFQVMLTLQNNATPEFAMAGLRVTPDVAELAAAKFDLLFSLTDHYTEDGAPDRVDGVLEYSADLFDHGTAEALTARFVRLLCAVAEAPDRPIGTVDLTDAAERERLLETWNGAALTTPPVTLPEAFEAQTRRTPDAVAVTDGSTNLTYAALNARANQLARLLLGRGIGPEQLVAVALPRSAELVVALLAVLKTGAAYLPLDPAHPAERLRFLLGDARAAYGITGSALVGDLPETDTRWLTPDSDEARREAGDQPEHDVTDAERTAPLRPEHPAYVIHTSGSTGTPKGVLVEHRALAAYLAFAREAYPGTAGTTLLHSPVSFDLTVTALYAPLTAGGRVRIAELSADAPAPGPYTFLKVTPSHLALLGATPPGPETAVADLVAGGEQLTGEQLAPWRRAQPQAAVVNEYGPTETTVGCVVLRMPPGATAGDGPLPIGRPVPGTRAYVLDERLRPVPPGVTGELYLAGAQLARGYLNRPALTAERFTADPFGGPGGRMYRTGDLARWSTEGELHFAGRADEQVKVRGHRIEPGEIETALLRHPAVARAAVVAREDRTSGTQLVAYVVPADGERSGDAAARAQLDEWQEVYDSLYGGDGHQDAEPAPFGADFRGWTSSYDGRLLPVEQMRRWREATVDRIRELRPRRVLEIGVGSGLLLSRLAPDCETYWGTDIAADAVDRLRTQVPERPDLAGRVELRVQPADDLTGLPEGFFDTVVINSVAQYFPDAGYLTDVLGTALGLLAPGGAVFLGDVRDLRLLRCMHTAVQLRRATPSADPAAVRRAVEQAVLLEKELLLDPAFFAALAARNPDVAGVDLRVKRGAYVNELNRYRYDVVLHKRSADGPTDLAAAPELRWGHDVTGPDDLARRLTTERVPALRVTGVPDERLAAEAGAARGLDEGRPLADVLALLGPAEQPTAPDLGPDTFHELGERAGYQVAVTRSGDDAELLDVLFTDPAVTALSGVHRPAAEPSHPLANDPAASRQHGELVASLTASLRAELPAHMVPAAVLVLTELPLTGNGKLDRAALPAPYLGSVEGSRGPSTPRERVLCDLFAQVLGLAAAGVDDNFFHLGGHSLLATRLISRIRTALGVELPVRALFETPTVAGLAARLDDEESRDATRPALRPMSRPDTLPLSFAQQRLWFLHRLEGPNATYNMPIALRMRGAVDRTALRAALADVAARHESLRTVFPVTDGVPRQLVLDADGARPELLVRHTDETGLREAVQAAARYPFDLAADLPLRAELFDLGEREHLLVLVVHHIAGDGWSMAPLARDLATAYAARTRGRAPSWTPLPVQYADYTLWQRALLGDETDPDSPFARQIAYWSETLADLPQELALPTDRPRPAVSTYRGEMLGFDLSPELHRRLADLARDTGTSLHMVLHAGLAALLTRLGAGTDLPIGSGVAGRHDEALDDAVGLFVNTLVMRTDTSGDPSFGELLRRVRETALNAYSHQDVPFEHLVEALNPVRSPNRHPLFQVAMVLQNTPEAGLALPGVEVTPEPAASGTSRFDLFFSVTERGSSGGVRGTLEYSTDLFDRRTAETLVERWARLLEAVTADPARPIGEADVLTPAEHARTARASGADGPQVPPATVPELFARQVARHPGALAVLSDSGTLTYQELNVRANRLAHLLLSRGIGPGHVVALAFPRGAGLIVALLATLKAGAAYLPLDTGYPAERLAFMLDDARPSLLLTGVAGQEIPGSGRLSRLDLDDGDGHLAGQPDTDPGAEQAGRPDPAHPAYVIYTSGSTGVPKGVAVTHAGAAALAATQADSFGAGPGSRVLQFASPSFDAAFWELCMAMLTGAALVVAPAERLLPGPDLAAVLAEHEVTHLTVPPVALGVMTPDQLAPVTCLVVAGESCPPQTAAAFAAGRRMVNAYGPTESTVCGTMSRPLTGDGAVAPIGTPITGTRAYVLDAALRPVPTGVAGELYLAGAGLAQGYLGRPGVTAERFTADPFGAPGARMYRTGDLARWNAEGELEFAGRTDDQVKVRGHRVELGEVEAAMAAHPEVAQAVATVREDGPGGTRLLGYVVPAADGPRFTEATGAAEAAQAADGAPERALGEWRQLYDTLYGTTQTAPFGEDFTGWNSTYDGEPLPADEMRQWRDATVARIRDLRPRRVLEIGVGRGLLLSKLAGECAAYWGTDLSAAVIESLTAQVAERPALAGRVELRTQPADDLTGLPEGFFDTVVLNSVAQYFPDAAYLTDVIGAALRLLTPGGALFLGDLRNLRLLPSLRAATGTTAPERELLVDPDFFPALPATVPEIGAVDLRLKRGAYHNELSRYRYDVVLHRAPLTAPARPFADAPTLPWHPGLGGTAEIAALLTERQPDRLRLTGIPNARLAGEAATGHLVTVDPETLCATGERLGFWTAVTWSGTAGDDCFDSLFAAQDAAAPDDTAPYGSAPVTGVYVPADGRTAPPSFTNDPAAAVAPSAALGSSVRAWLRRRLPEYLVPSVVIPLDVLPLTPSGKADRTALPAPETTAGTTAGRPPATARERLLCDIFADVLGLPSVGADDDFFDLGGDSIVSIQVVSRARAAGMAIGPQDVFRHKTVAALAVAAGTSDAAPAATEAPQAGIGVVPLTPDLHALRERDADPGTGYEAMTVPAPAGLGAEALEAALQAVLDHHDMLRARLTSGGPVAADGPGWLLEVRPPGTVRAADHVRRTGTAPHEAAVAEEERAAAARLAPESGVLLQAVHFDAPPGSPGLLLLVLHRLVTDPQTWRILLRDVTDACEAAADGRQPRLAPVGTSFRRWAQHLRTQARDPRRTGELSRWTGMLAGPGSPDDAPDARATGNPDGIPAHEAGDAMLTTLLSAERTGQVLTDVPAAFHADAEDVLLTAFALAVADWQRRSSAADGTVLADVRRPGRVPLGEGIDLSRTVGRCTTTHPVRLDLGDPGGEALREDGGHAAATALKRVKEELRSLPDGGLGHGLLRHLHPGTAPELAALPVPRFAFHYLGELPQAPSGPDPAGEPACYWAEVAITAVDGPGGPRLAATWRRAHAPLTGRDLNDLATAWTRALETLAAQAARPGAGGRTPSDVPLVRLSQDKLEQLEAAWRRSQ

SEQ ID NO: 68 - protein, 2376 aa, Streptomyces sp.
VTQPELEDILPLAPLQEGLLFHALYDTAALDLYTLQSAYDLDGDLDATALREAARTLLRRHAALRAGFWHEGLDEPVQIVPREVALAWQQADLTGTAPEEREAEAERIAAEDRLRRFDPARPPLLRFTLLRLGTERHRFVVTAHHLVLDGWSVQLLLEELFQLYVRGGREDGMPPVARYRDYLAWLAGQDRAAAEDVWRRALKGIEEPTRVAPADPSREPALPGQILRDLPDGLANALDALARRERLTLSTVFLGAWAVVLGRLTGKDDVVFGTTVSGRPPQVPGVERMIGLFANTLPVRVRLDPEQPLNGMLARLQEEQTHLLACQHVGLSGIQRTLGSGELFDTVTVFENLPAGQSGLAATAHGGGPRVTASRSQGVTHYPLNFMVMPGRPLRLRLDHQTGLFDRDAAEAIMARVVRVLENLVADPGRRLGELEILAPAERARVLGEWSGTAGEPPAELMPQAFAARVATTPDAVALVCGEVSWTFAELDARADRLARRLAARGAGPDALVGLCVRRSADMIAAVLAIWKAGGVCVPLDPGYPAERLAFMLADAAVPLVLTERELAGRLGEDRAASLVLLDDDTPSETGVALRTPPLHGDHAAYIVYTSGSTGVPKGVVVSHLGLANLYADHRDRVFAPAADAVAASAGRRLRVAHTASVSFDASMGQLAGLVAGHELHVVDEDTRRDATALLAYVRERSVDYLGIAPSLMRELTALGLLDGPGHRPAVILLGAEAVDAALWEALRAHPETEVHNYYGPSECTVDVVGALVRDSARPVIGRPVANTRAYVLDARLRPVPPGVVGEVYLAGIQLARGYLGRPGLTAARFVADPFGTPGTRMYRTGDLARWTADGRLDFAGRTDDQVKLRGYRVEPGEIATVLARHATVAHAAAAVREDRPGDQRLTAYVVPAPGARAHAEELREFCAGTLPEFMVPAQIVVLDALPMTPSGKLDRRALPAPGARTTGGREPRDPVEDILCGLFAEVLGVDRVGVDDGFFTLGGHSLLATRLVSRVRAALGAELAVRDVFEAPTVAGLAARLGTPREDTRRRPPLRPAGERPAELPLSFAQRRLWFLHQMEGPTPTYHMPFALRLTGGLDVAALRAALNDVVARHEVLRTVYPDTGGTPRQHVLGPAEAPAPLPVRTVAEDALAAALADAYARPFDLTREPPLRAELFALGPDDHVLLLVLHHIAGDAWSNLPLARDLSAAYAARRTGEAPAWAPLPVQYADYTLWQRGFLGEESDPDSALSRQVAYWRTALDGLPAELELRHDRPRPEVSDHRGGTVPLHVGPATHARLAGLARAHGASLFMVVQAALAALLTREGAGTDIPIGTPVAGRTDEGLDDLVGCFVNTLVLRTDTGGDPVFTDLLDRVRRTGLAAYSHQDVPFEHLVEVLNPARSAARHPLFQVLLSLHHAADLPVELPGLRVRAEETRSRSAKFDLTFRLAERKGPDGGPGGLEGGVEYSTDLFDRSTVEGLAARFARLLDAVAADPGRRIRRLDVLSPAERERVLTGWNATERPLPPVTLPELFAAQVARTPDAVAVVGERDTLSYAELDAWAQRLARALTGRGAGPERVVAVVLPRSVELVVALLAVLKSGAAYLPIDPALPAERIRLLLDEARPVYVLDDADGVRNPRPETAGTARPVPLSPSHPAYVLYTSGSTGAPKGVVVPHAAVVNRLLGIQEEFGLEPSDICLYKAPAAFDASVWELFWPLTTGARIVVAAPDGHRDPAYLAALIREHAVTTVHFVPSMLRAFLDEPGAARCTSLRRVQTGGEPLPAELRDRATHLLGAPLNHLYGPTETTIHATTWSCGRPSAAAPDTTAARPVPIGRPVRNTRAYVLDAALGPVPPGATGELYLAGVQLARGYLARPGLTAERFIACPYGAPGEQMYRTGDLARWTANGVLEFAGRADDQVKLRGFRIELGEIEAALSRHEDVAAAAVVLREDRPGEQRLVAYVVPAPETGPHPADLRRHAALHLPEFMVPAAVVVLAALPLTSSGKLDRRALPEPGGPLTVTAKRPARTPEEEVLCGLFAEVLGLTGTGIDDDFFDLGGDSLAAIRLVRRIREAAGVELSVSDLFAAPTVSRLMDRLRLGVRGDAFDVLLPLRESGGLPPLFCVHPAGGLSWAYTGLLRHLGPDRPVYGLQARGLARPETYPTSMDELVTDYVRQLRSVRPTGPYHLLGWSFGGLAAHAIAVRLQAEGEKVAFLALLDSYPRDPEAAVPPVEERDVLATLLDFAGHGEDVTDAEQLRVEDVMEILRRGGGPLADIEERRVGALADVFVNSAELASAHAPGRFDGDLLHFTATAGRPDGAPTAAVWEPFVTGRVRRYTVDCTHHAMCRPEPLAEIGRVVAERLDGADRPRRQDGPNR

SEQ ID NO: 69 - protein, 76 aa, Streptomyces sp.
MTNPFENADSTYLVLVNDEGQHSLWPSFAEVPAGWTVAHGEDTRDACLAHVEEHWTDMRPKSLVRVMDEAGAPSAR

SEQ ID NO: 70 - protein, 371 aa, Streptomyces sp.
LTAADREERDTAVLTYQEPPAGGGAWAETVELTPQEAEQFRKVADEITGMLPHGKPAGPGQLDDPGLLAEIEVAARWLPPRLARALSRFRRQGNPHGVLLVRNVPLDADLPPTPADGAAPVWSRLGTATVGQLAVVSWLGDVIAYADEKAGRLVQDIVPVSGAEECQENSGSVLLELHTEDGFHPYKPDFVTLLCLRADHERRARTTYGAITRALPRLSAECVRVLRRPLFRIHFSSSFGGPGAGAKGSGTGTGTGVVSPPLPVLSGPDGDPEIVADFHAMEPLDETAARAFGELRDAMLAVLDGAVLEAGDLIVVDNRTAVHGRTAFRPRYDGQDRWLRRCFAVADLRPSRAARTPGSQVCAPLKPEGGP

SEQ ID NO: 71 - protein, 56 aa, Streptomyces sp.
VTADRRAVAPAAAGRAPGRQASGCPTPGRPTSTPEAAVIGTDMSAQVRALAAKGGR

SEQ ID NO: 72 - protein, 120 aa, Streptomyces sp.
MEPMEPMAGNDPHIHVQAQAGRDDAAARNLIAATFGLVSNLPSVVATGCGLRVPYAMTSSRPDRVTCLACREHARQAHLRFAVEVERLSRMLGSVITPAQGKLAADRHRDLARRFSDDEG

SEQ ID NO: 73 - protein, 129 aa, Streptomyces sp.
MRIYVTSVFVDDQAKALSFYTDKLGFVKKHDVPLGGEARWLTVVSPEDPDGTELLLEPSGHPAVQPYKTALVQDGIPATSFAVDDVRAEFERLRGLGVTFTQEPLEMGTVTTAVLDDTCGNLIQIVHTA

SEQ ID NO: 74 - protein, 97 aa, Streptomyces sp.
MAEDVFKALADPTRRRILDELAERNGQTLFEICARLVTKHGLGLSRQAISQHLAVLESAGLVRTRRQGRYKFHDLDTAPLEHIMTRWLSTDTPESTP

SEQ ID NO: 75 - protein, 230 aa, Streptomyces sp.
MHPSVRNNARMEDRIAPTPLLPVRGAARCAVAALVLAGAAWAARALWQIRLATAGMPASGPPDQGEGKHRPLTALEDGYHIVTTVGSAATVLCAITFLIWLSRVRDNARALSGQSPRYARPWLYAGWIVPVANLWVPRGVVADIHRASAPGERLPHAVNWWWGLWLVGMLSGTGLMYTVPTDDVIERAYQAVYPLLAADAAVVAAAVAGVFVVRALTAAQRRRGVDSVRA

SEQ ID NO: 76 - protein, 337 aa, Streptomyces sp.
VHQLTGAAGSAVSEATSAAYYASIAQAEESGDGTVRHRLRPEVGDGTIEVTSLRSGFDIVRYDVAFAGGHQVGFRFPGDHFELEHCVDGRMHISEAGAGAGDLRARSVALSPRGAIDGVVAHGDGERYRAVSVTGTWSGLESYLGGLGAGALPPTGPESVARERYLGRSAALRPAAGPLEEIFLHRRSSPGRLLFMESRLVAALAALIDGLASDASPADARDRAAPALADHETAALRRVPELLWRERHAPPTLAELARASSMSVRRLTTGFRALYGTSVMEYHRRHCLDRAAALLVETDWPVSRIAHEIGYASAGNFGYAFRRDRGTSPARFRRAHS

SEQ ID NO: 77 - protein, 394 aa, Streptomyces sp.
MVLGGLSAVSPFAIDMYAPGFPEIVTSFGTSKTAVQLSLTACLVGLAVGQVVLGPVSDATGRRRLLLAGSGLFSVLSLVCAAAPSIAVFDAARLLQGVAGGAGIVIGRAVVSDLFTGAPAARRLTTLSVIGSTAPVLAPVAGGLVLGVGSWRLVFVALAVTGLLLLTAIWAWVPESLPAERRRTGGLPAVLRAMGGLLRRREMAGHLLVMGCGLAALFAYITGSPFVFENGYGLSPTQYSLVFATNAVGTVLAGTVFGRLAGRVRLNLLLTVGVAVTVVALVTLVTLLAVGISTFLTTWACLFGLTSGFGLLLPASTTIVLAVGGDAPGAASGMLGGAQFVLGAAAAPLPGVLGDTTAMSTAVVVLGCVLLSATALLTLARPWEGHGEPAGSPE

SEQ ID NO: 78 - protein, 186 aa, Streptomyces sp.
MHRSIDLHHTADTFEKCAQLCRSAREATDRAVRMRAEAAEARLRAVRMRRTMERMKAGHRSDDAPGCRRLPATETRRGATARAAQLALLTAFVHARLDEEGGTADLFHETGCPGAGASECHCPVPERVRHDVAVRHGLARDCEEALHDPDHDACDWPHSELAALLRLKVLALPYELHRQWREEWRP

SEQ ID NO: 79 - protein, 252 aa, Streptomyces sp.
MNQKREIAALIGDVFGPAAVGSYLYGSAVLGGLRPASDLDVLVVSRRRATVPQRRALLDGLLGISGFTGGARPVELTVVVQDQVHPWRYPPICDFLYGEWLRDTYAAGVVPEPEPLPDLALLVTMALAGDHPLTGPPPAQVLDPVPWADVARASVHGIPGLLDEVDSDTRNVVLTLARVWTTLATGEIRTKDAAADWALGHLPPRHRPVLRHARELYLTRRYSEESWSDDLRSRVRPHVDHVLAEIRELPYA

SEQ ID NO: 80 - protein, 335 aa, Streptomyces sp.
MTEAGRSVPPRRRLPFRQFVLKVHSRCNLACTYCYVYEGPDQSWRERPARVAEETVRRTAERIARHVTTHGLRSIRVDLHGGEPLLLGPAALIDHARAIRNAVPGDCAVAVSVQTNGTLLTEDAVRRCSAAGISVGLSLDGGSARLNHRRVDRAGRSSWPAVRRAAGLLAGVPDAYGGILCTVDVEADPAEVYRSLIALAPTGLDFLLPYGNWSAPPPGTGPFAGPVGRAARPVPYGAWLARVFDLWWDTPPGRRAVPVRLFGEIVALLLGAASRSEAVGLSPLAVVVIDTDRYLHGSGFGHPSVYCADLEHLIRHIGERLQEEVRGVPGDPVRP

SEQ ID NO: 81 - protein, 220 aa, Streptomyces sp.
MNAEAEAQAQAAAAARTESPVQAQTAADLIGSEPAATGWPDVPGDTALTIKVPEADPLVRAGFPAHVTVLYPFLHESRIDATAQHDLTRLFAGHDAFTLTLDSFGRYPGVLYLDPWPHAPVTALTKDLTRRWPEAVPYRGIFGAGLAPHLTVANHEGPATQEAAYDALQAELAPSLPLTCSVREVHLIVWDGRRWRDRTAYRLGPPAPCRLTLSQRQRFY

SEQ ID NO: 82 - protein, 265 aa, Streptomyces sp.
MRIGELAGIVGVTTRAVRHYHRTGLLPEPPRQPNGYREYALRDAIELARIRRLVELGLSLDEVKDVLADDAGKDLVEILTELDADLARQEEAIRQRRARLARLFEQAERTGRLPEEAPVSPELAAVFDDMARASAQVPGPEPAMAAKERQLLALLETGSPGGDRGWFDGLVHAFRADPAAMRRAYDVYARMDELAEAAEDDPRVELTARAIVDSIPDEALRAMRLPADGAEEAGDGFSDAFFSDFAPAQAAAVYRAIELLTERAG

SEQ ID NO: 83 - protein, 307 aa, Streptomyces sp.
VNTLRKAVRWSFAALVPGELILVLCVAGGVRIAPAVRLATEPAMLTLAAAAAVLIALDHRRHRRAGLGHRAAFAAAIADTVPAPVRKLTAHEVFLSTSFLRWVTRRGPHGVRDGDLSVPYAPGQTATMYGFLFVCVVETVGFAFLIPWPVVHAVTLVLDIWGCYFVIALHASCVVRPHVVGADGSLRLRHGALLDIRIPAGRVAAVRREQKFPDGKLAVVDKNGVADMAVAGRTTVLVELTEPVRFVRALGKTAEARAFRFYAEDPAAVVTALKAGAASPCLASTRPSASGPDASASHAASQAGSPG

SEQ ID NO: 84 - protein, 338 aa, Streptomyces sp.
MHASNSPAGTLGSPTPVLSVGPVVLPVPGRAVDLQLRISAPVTGSDLPVVLLSHGQGYSHHLSSLNGYAPLANYWAAHGFAVIQPTHLSSRTLDLDPATPGAPLHWRSRAEDMRHILDRLDLIEAAVPQLPGRLDRERIAVAGHSMGGHTASLLLGARLTDPDDGTVVDLAEPRIKAGVLLAAPGRGGDALTASVAEAFPFFATTDFSRMTTPTLVVAGDQDDTAHLTVRGPAWHADPYVLSPGPKSLLTLFGAEHGLGGVSGYDVAETTDESPERVAAVQRLTWAYLRSRLYPGDPAWEAACEALASGPDALGRVEAKQGDAAPAFSAVTTAAGSSA

SEQ ID NO: 85 - protein, 63 aa, Streptomyces sp.
MNTGNTTDTPAAARQAKFGKLPERIRFEDMVTERPSPESRTPNAYTPEGTWNHFSCLALDLGL

SEQ ID NO: 86 - protein, 187 aa, Streptomyces sp.
VNTDHVFVCGHPALDFAATLRARRTVRFEMFATPERLEAWYVESGLVDSVTPARPSDVERAKSVREAAYELITARRLGEPFGARALDVLNKAARKPSAAPQLTSAGRRTDATPEEALSAVARQAVELLSGPDVPLLKECGNPECTRVYIDRSRGARRQWCGMESCGNKIKAAAYRARKRGAPATTAR

SEQ ID NO: 87 - protein, 585 aa, Streptomyces sp.
MPRPALLAHDLVRTLGTRRVLDGVSLTVSPGRRVGIVGENGVGKSTLLRLLAGVDEPDSGQVTRPDDLGFLHQEMPFDGATTIARVLADALREARAALAELDRLTRLLAGTPDDHPGHAELLDAYGKQLEHAQESEAWDADRRAAIVLDGLGLGGFGHGRTLGSLSGGQRGRLALAALLVRRPAALLLDEPTNHLDDEAAAFVEEQVRVQPGVVVAASHDRAFLDAVCTDLIDLDPAVHGPVRFGGNYSAYRAEKQAERERWEQRYAAEQEELEALRRSAGVTARRVAPDRGPRDNEKMGYGHRAGRVQQQVSRRVRNAARRLDELERTQVGRPPKPLRLHAGALTGALVDGAPADSGQVHGATADSAPVDSPLVALCDARVPGRLDVARLDIPAGDRLLVTGDNGAGKSTLLSLLAGRLDVEREGAVRRRPGLTVGLLAQDTVFEHPDRSARDTYTHVLGAERAEAVPLGSLGLLGVTDLDQPVGRLSAGQRRRLALALLVAHPPHLLLLDEPTNHLSPRLCDELEDALGAGPGAIVVASHDRWLRGRWTGRRLRLRAGRLAPPDDVYGIRSTGGRLRPGTSDR

SEQ ID NO: 88 - protein, 544 aa, Streptomyces sp.
MSDAFVVCSSLSFSWPDDTPVFQDLSFTVGTGRTGLVAPNGSGKSTLLKLIAGELRPAAGTVTVSGTLGHLPQSLPLAAGLTVAEVLGVAEVIRALDAVESGDVDEKHFATIGDDWDIEERTRAQLDRLGLTELTLDRGLSTLSGGQIVSLGLAAQLLKRPDVLLLDEPTNNLDLDARHKLYDVLEDWNGCLLLVSHDRALLDRMDRIAELEPGALRLYGGNFTAYEEAVQAEQEVAEKNVRNAEQELKREKRELQQARERADRRASNASRNLKNAGLPRIFAGNMKRGAQESAGRAGQMHASRVSEAKARLDEAGRAVRDEQRITLDLPDTDVPAGRNLFLGEGLRVSHGERPLFADGGVDLTIRGPERIALTGPNGAGKTTLLRLLQGGLEPDAGQIKRADSRIAYLSQRLDVLDLSRTVAENFAAFAPQRTAAERMNLLARFLFRGSRAHLPVQVLSGGERLRATLACVLCAEPAPQLLLLDEPTNNLDLVSAGQLESALASYQGAFVVVSHDERFLARIGIDRWLRLADGELRETAVPEV

SEQ ID NO: 89 - protein, 320 aa, Streptomyces sp.
MHAIRMDRHGGPEAMNWTALPDPAPGKGEVLVRLAVAGVNFMDVGARTSGGPGWAAPAILGDEGAGRVTALGEGVEGFAPGDRVAWFYHPGSYAELLAVPPESLVKLPDEVSDETAAAVMMQGLTASHLTTETYPVGPGDTAVVHAAAGGVGLLLTQMIKARGGRVIGLVSREEKVAAAREAGADHVLVSRGGGFEDRVRELTDGMGAQVVYDGGGSATFRSSQRALRPHGVHAYYGPFMGVPSVAPTDLPDSIFLTYPVVHHHVPTRAALVQRTGEVFDLIRDGRLTPRIGGRYALSDAARAHADLESRRTMGKLLLVP

SEQ ID NO: 90 - protein, 199 aa, Streptomyces sp.
MPLTRKGAATRQRIVEGAASEMRRLGIFALTLDDVMRRTATSKSQLFHYFPGGKDELLPAVARHEASGVISDQQPELASLNSWSAWRSWRDKIVERYREQGPNCPLNVVMGQLGPTTEGAQAVVRELYARWEGELAAGIRGMQSQGEIDAALDAGRGARALLAGIQGGVLIHLATGSLEYLKAALDQGIEFLRRPAARA

SEQ ID NO: 91 - protein, 122 aa, Streptomyces sp.
MDGLTESNNGVPEPHSGWTFVTNHARVLAAIADDRHARIRDIAAHCRLTERAVQKIISDLERDGYLSHTREGRSNTYRIDPGKVLRHPAEAGLTVASLLSLLVQDEQDRSTQPDGRRHPSTA

(19) United States
(12) Patent Application Publication
(10) Pub. No.: US 2026/0347403 A1
(43) Pub. Date: Nov. 5, 2026

(54) FUNGICIDAL COMPOUND

(71) Applicant: SYNGENTA CROP PROTECTION AG, Basel (CH)

(72) Inventors: Stephane BIERI, Stein (CH); Philip SIDEBOTTOM, Bracknell, Berkshire (GB); Dimitrios PAPASOTIRIOU, Bracknell, Berkshire (GB); Dianne IRWIN, Bracknell, Berkshire (GB); Lauren RAY, Bracknell, Berkshire (GB); Peter VAN DE VONDERVOORT, Echt (NL); Leon COULIER, Echt (NL)

(73) Assignee: SYNGENTA CROP PROTECTION AG, Basel (CH)

(21) Appl. No.: 19/118,440

(22) PCT Filed: Oct. 5, 2023
(86) PCT No.: PCT/EP2023/077604

§ 371 Date: Apr. 4, 2025

(30) Foreign Application Priority Data
Oct. 7, 2022 (EP) 22200225.5

(51) Int. Cl. C07K 7/56; A01N 43/36; A01N 43/72; A01N 63/28; A01P 3/00; C12N 1/205; C12R 1/465

(52) U.S. Cl. CPC C07K 7/56; A01N 43/36; A01N 43/72; A01N 63/28; A01P 3/00; C12N 1/205; C12R 2001/465

(57) ABSTRACT

The present invention relates to a compound of Formula (I) and a composition containing, a process of producing the compound and a method of using of the compound and the compositions to prevent or control fungi in plants.

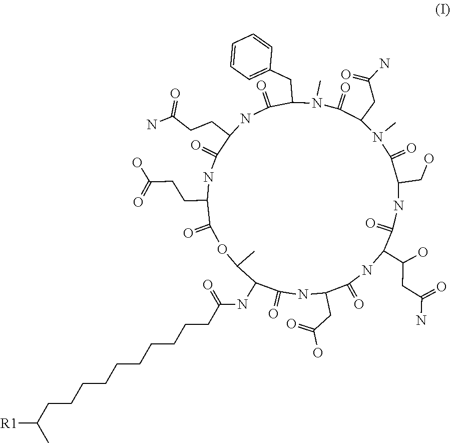

The present invention relates to a novel compound which has pesticidal activity. The invention also relates to compositions comprising the compound, to a process of preparation of the compound and to the use of the compound or the compositions in agriculture or horticulture for preventing or controlling phytophatogenic infestation of plants, harvested food crops, seeds or non-living materials.

### BACKGROUND

Fungicides are widely used in agriculture to protect plants against damage caused by fungi. Fungicides may be from chemical origin or biological origin. Due to some negative effects of chemical fungicides on the environment, there is a growing need for fungicides from biological origin, for instance microbial origin. Known microorganisms that produce antibiotics against fungi are actinomycetes, for instance Streptomyces sp. A very well-known species is Streptomyces natalensis that produces the antifungal compound natamycin, which is used in food and crop protection. In U.S. Pat. No. 5,356,624 a Streptomyces rimosus strain is disclosed that was found active against several wood-degrading fungi. In WO2022/038180 new Streptomyces sp. are disclosed that produce several known antifungal compounds such as streptimidone, natamycin (pimaricin), or albofungin. The extracts of these bacterial strains were found active against well-known plant pests such as Fusarium graminearum, Zymoseptoria tritici, and Puccinia striiformis.

Another compound that is produced by Streptomyces sp. No. AC-69 is Lipopeptin A which is known to be active against some phytopathogenic fungi (Tsuda, Suzuki (1980), The Journal of Antibiotics Vol. 33, no2, p. 247-248.

Due to development of resistance by fungi against fungicides, regulations by governments and societal pressure there is a continuous need to look for new compounds that have fungicidal activity from biological origin.

### SUMMARY

The present invention relates to a compound according to Formula (I)

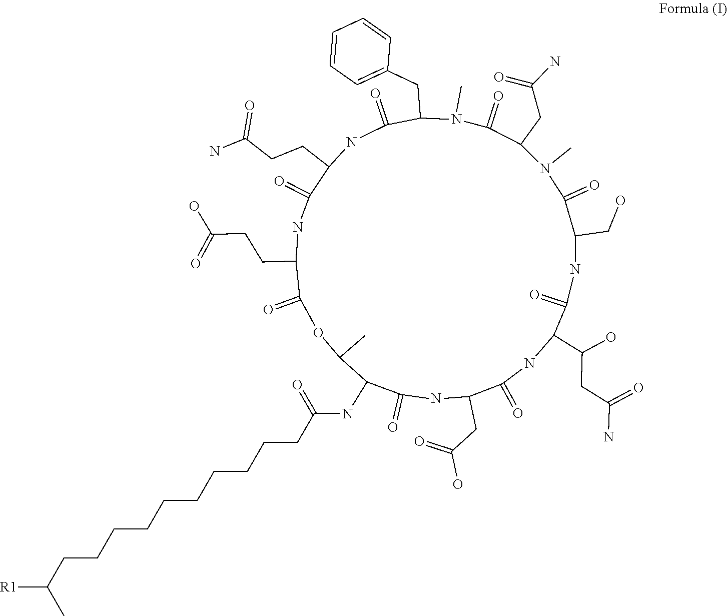

or a salt thereof, wherein R1=CH₃ or C₂H₅

Surprisingly, it has been found that the novel compound according to the present invention has a surprising level of biological activity for preventing or controlling phytopathogenic microorganisms such as fungi. Biological activity as used herein includes fungicidal activity. In a second aspect the invention relates to a composition comprising a compound according to the present invention and a microorganism that is able to produce a compound according to the present invention.

In a third aspect the invention relates to a process for producing the compound, or a composition according to the present invention, comprising cultivating a microorganism in a suitable fermentation medium under conditions that allow production of the compound.

In a fourth aspect the present invention relates to a method for controlling or preventing infestation of a plant by a phytopathogenic microorganism, wherein an effective amount of the compound according to the present invention or a salt thereof, or a composition according to the invention as disclosed herein, is applied to the plant, to a part thereof or a locus thereof.

According to a fifth aspect of the invention, there is provided the use of a compound or a composition according to the present invention as a pesticide, preferably a fungicide. According to this aspect of the invention, the use excludes methods for the treatment of the human or animal body by surgery or therapy.

### DETAILED DESCRIPTION

The present invention relates to a compound according to Formula (I)

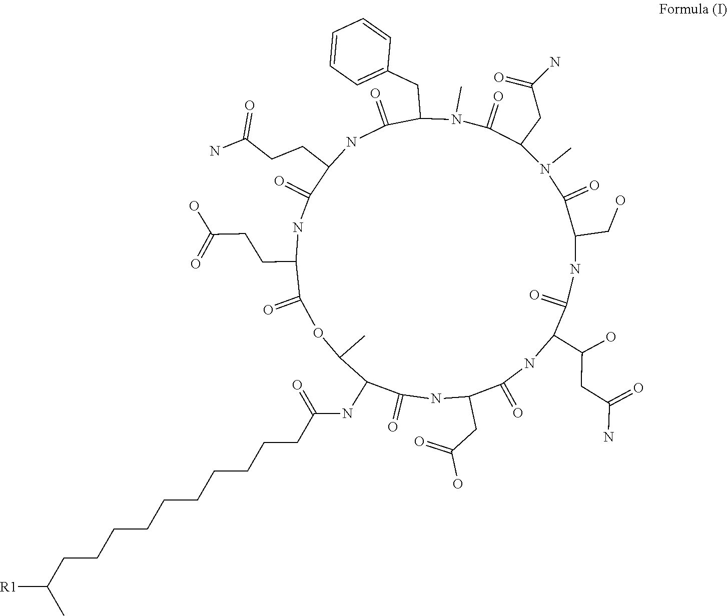

or a salt thereof wherein R1 is CH₃ or C₂H₅.

The compound according to the present invention comprises or is a lipopeptide.

Accordingly, the compound according to the present invention comprises a compound according to Formula I(a) and/or a compound according to Formula I(b) or a salt thereof.

A compound according to Formula I(a) comprises structural formula

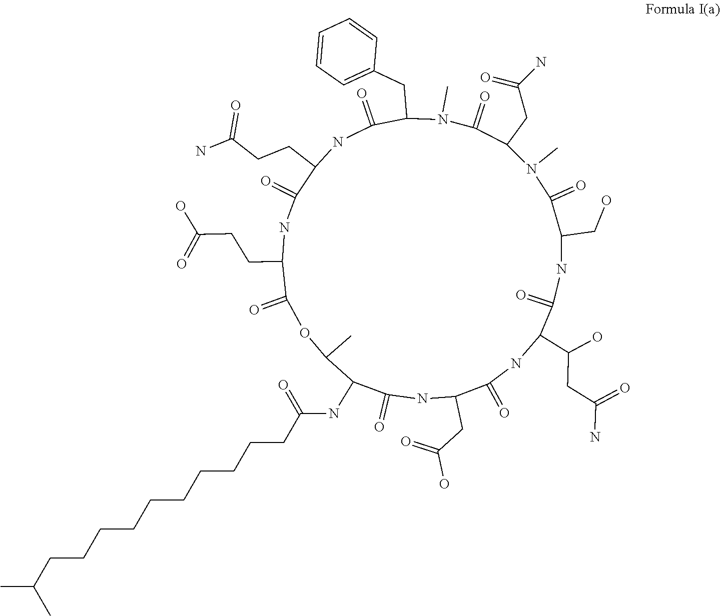 [SMILES: CC(C)CCCCCCCCCCC(=O)NC1C(=O)NC(CC(=O)O)C(=O)NC(C(O)CC(N)=O)C(=O)NC(CO)C(=O)N(C)C(CC(N)=O)C(=O)N(C)C(Cc2ccccc2)C(=O)NC(CCC(N)=O)C(=O)NC(CCC(=O)O)C(=O)OC1C]

or a salt thereof.

The compound according to Formula I(a) comprises a molecular formula C₅₅H₈₅N11O₁₉ and an exact mass of 1203.602 g. The compound according to Formula I(a) has a solubility in DMSO of above 10,000 ppm. The compound according to Formula I(a) comprises or is a lipopeptide.

A compound according to Formula I(b) comprises structural formula

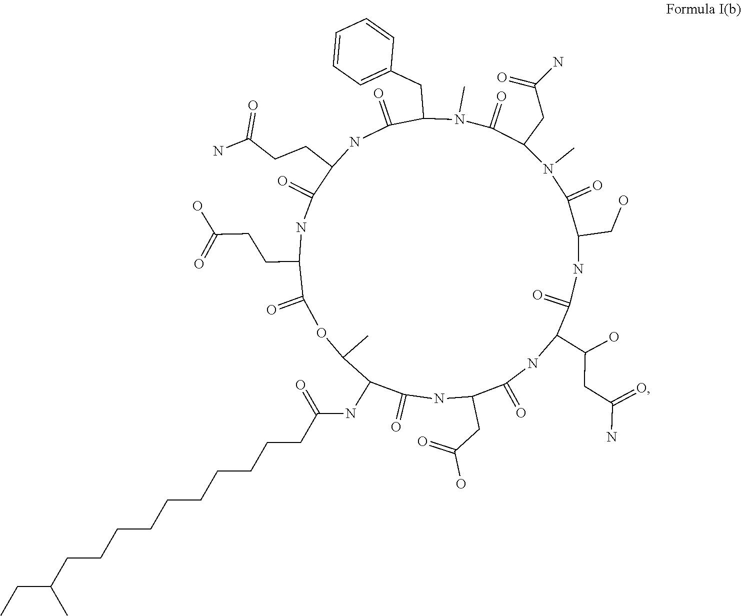 [SMILES: CCC(C)CCCCCCCCCCC(=O)NC1C(=O)NC(CC(=O)O)C(=O)NC(C(O)CC(N)=O)C(=O)NC(CO)C(=O)N(C)C(CC(N)=O)C(=O)N(C)C(Cc2ccccc2)C(=O)NC(CCC(N)=O)C(=O)NC(CCC(=O)O)C(=O)OC1C]

or a salt thereof.

The compound of Formula I(b) comprises or has a molecular formula C₅₆H₈₇N₁₁O₁₉ and an exact mass of 1217.618 g. The compound according to Formula I(a) has a solubility in DMSO of above 10,000 ppm. The compound according to Formula I(b) comprises or is a lipopeptide.

In one preferred embodiment the compound according to Formula I is an isolated compound. The wording ‘isolated’ with reference to the compound means that the compound has been isolated from it's native environment.

In one aspect, the present invention relates to a composition comprising a compound according to the present invention and a microorganism that is able to produce a compound as disclosed herein. The compound or the composition comprising the compound according to the present invention and a microorganism that is able to produce a compound according to the present invention is applied to plants or to parts thereof to cure or protect plants against diseases caused by phytopathogenic microorganisms such as fungi, bacteria or viruses.

Surprisingly, it has now been found that the compound and/or composition according to invention has an advantageous level of biological activity for curing or protecting plants against diseases that are caused by infestations of phytopathogenic microorganisms such as fungi, bacteria or viruses.

Surprisingly, the compound and/or composition according to the present invention has an advantageous fungicidal activity against various phytopathogenic fungi.

Preferably, the compound and/or composition according to the present invention have fungicidal activity. Accordingly, the compound and/or composition according to the present invention preferably is a fungicide.

The term “compound having fungicidal activity” or “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

The compound and/or the composition according to the present invention may be produced in any suitable way, preferably the compound and/or the composition of the invention is produced by cultivating a microorganism in a suitable fermentation medium that allows producing the compound and/or composition of the present invention. The microorganism, preferably is a Streptomyces sp.

A microorganism able to produce a compound or composition according tot the present invention comprises or is a microorganism comprising at least one nucleotide sequence that encodes a protein that has at least 80%, 85%, 90%, 95%, 96%, 97%, 98%, 99% identity or 100% identity to an amino acid sequence according to SEQ ID NO: 47 to 91, preferably an amino acid sequence of SEQ ID NO: 67 and/or SEQ ID NO: 68.

Preferably the microorganism comprises a nucleotide sequence(s) that encode(s) at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at last twenty four, at least twenty five, at least twenty six, at least twenty seven, at least twenty eight, at least twenty nine, at least thirty, at least thirty one, at least thirty two, at least thirty three, at least thirty four, at least thirty five, at least thirty six, at least thirty seven, at least thirty eight, at least thirty nine, at least forty, at least forty one, at least forty two, at least forty three, at least forty four, at least forty five of the amino acid sequences of SEQ ID NO: 47, SEQ ID NO: 48, SEQ ID NO: 49, SEQ ID NO: 50, SEQ ID NO: 51, SEQ ID NO: 52 SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61; SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, or SEQ ID NO: 91, preferably SEQ ID NO: 67 and/or SEQ ID NO: 68, or (an) amino acid sequence(s) which has/have at least 80, 85%, 90%, 95%, 96%, 97%, 98%, 99% identity thereto.

A microorganism able to produce a compound or composition according tot the present invention comprises a microorganism comprising at least one nucleotide sequence that has at least 80%, 85%, 90%, 95%, 96%, 97%, 98%, 99% identity or 100% identity to a nucleotide sequence of SEQ ID NO: 2 to 46, preferably to SEQ ID NO: 22 and/or SEQ ID NO: 23.

Preferably the microorganism comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at last twenty four, at least twenty five, at least twenty six, at least twenty seven, at least twenty eight, at least twenty nine, at least thirty, at least thirty one, at least thirty two, at least thirty three, at least thirty four, at least thirty five, at least thirty six, at least thirty seven, at least thirty eight, at least thirty nine, at least forty, at least forty one, at least forty two, at least forty three, at least forty four, at least forty five of the nucleotide sequences of SEQ ID NO: 2, SEQ ID NO: 3, SEQ ID NO: 4, SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16; SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46 or which nucleotide sequence(s) has/have at least 80, 85%, 90%, 95%, 96%, 97%, 98%, 99% identity thereto.

The microorganism able to produce a compound according to the present invention may be a naturally occurring microorganism or a recombinant microorganism. Recombinant microorganisms can be produced by methods known to a person skilled in the art. A recombinant microorganism may be produced by transforming the microorganism with at least one of the nucleotide sequences that encode a protein of at least one of the amino acids sequences according to SEQ ID NO: 47-91, preferably an amino acid sequence of SEQ ID NO: 67 and SEQ ID NO: 68, preferably at least one of the nucleotide sequences of SEQ ID NO: 2 to 47, preferably at least one of the nucleotide sequence of SEQ ID NO: 22 or 23, or a nucleotide sequence which has/have at least at least 80% identity, preferably at least 85%, preferably at least 90%, preferably at least 91%, 92%, 93%, 94%, 95%, 96%, 97%, 98%, preferably at least 99% identity thereto.

Preferably, the microorganism in the composition or in the process of the present invention as disclosed herein is a bacterium of the genus Streptomyces, preferably the bacterium is a Streptomyces chrestomyceticus, S. rimosus, or S. paromomycinus, or S. monomicini. Preferably, the composition comprises Streptomyces sp. Saigon413 deposited with the Westerdijk Institute under accession number CBS149411. Preferably, the microorganism is a Streptomyces sp., for instance Streptomyces sp. Saigon413 deposited with the Westerdijk Institute under accession number CBS149411, wherein the Streptomyces sp. has a 16S RNA sequence which has at least 98%, preferably at least 98.2%, 98.4%, 98.6%, 98.8%, preferably at least 99%, 99.2%, 99.4%, 99.5%, 99.6%, 99.7%, 99.8% preferably at least 99.9%, or has 100% identity to SEQ ID NO: 1.

Preferably, the microorganism in the composition or in the process according to the present invention, for instance a Streptomyces chrestomyceticus, comprises a genome sequence which has at least 90%, 91%, 92%, 93%, 94%, 95%, 96%, 97%, 98%, or at least 99% identity or 100% identity to the whole genome of Streptomyces chrestomyceticus NRRL-3672 or to the whole genome of Streptomyces sp. Saigon413 deposited with the Westerdijk Institute under accession number CBS149411. In one embodiment the composition or process according to the present invention comprises a Streptomyces chrestomyceticus which is Streptomyces sp. Saigon413 deposited with the Westerdijk Institute under accession number CBS149411.

As used herein, the terms “percent identity,” and “percent identical” refer to the relatedness of two or more nucleotide or amino acid sequences, which may be calculated by (i) comparing two optimally aligned sequences over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100 percent to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present invention, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100 percent.

The present invention also relates to a microorganism which is a Streptomyces sp. Saigon413 deposited with the Westerdijk Institute with accession number CBS149411.

It was surprisingly found that Streptomyces sp. Saigon413 deposited with the Westerdijk Institute with accession number CBS149411 has advantageous properties as compared to Streptomyces species known in the art.

The compound of the present invention, or a composition comprising the compound of the present invention can be used in the agricultural sector and related fields of use, e.g., as active ingredients for controlling phytopathogenic microorganisms. The compound according to the present invention is distinguished by excellent activity at low rates of application such as from 2 to 250 ppm, for instance from 10 to 200 ppm, for instance from 20 to 100 ppm, by being well tolerated by plants and by being environmentally safe. It has very useful preventive properties and can be used for protecting numerous plants. The compound of the present invention can be used to inhibit or destroy phytopathogenic microorganisms that occur on plants or parts of plants (fruit, blossoms, leaves, stems, tubers, roots) or different crops of plants. The compound may also protect those parts of the plants that grow later.

The compound according to the present invention and/or a composition comprising the compound according to the present invention can be used as such or formulated with an auxiliary, preferably an agricultural-acceptable auxiliary. Known formulations in the art are for instance emulsifiable concentrates, coatable pastes, sprayable or dilutable solutions or suspensions, powders, dusts, granulates and encapsulations.

Accordingly, in one embodiment a composition comprising a compound according to the present invention as disclosed herein further comprises an auxiliary. Preferably the auxiliary is an an agricultural-acceptable auxiliary.

Suitable auxiliaries are known in the art, and include for example solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers include, for example water, organic solvents, oils of vegetable or animal origin, cyclic and aromatic hydrocarbons, alcohols, esters, fatty acids, a glycol or any other suitable liquid carrier known in the art. The solvent or liquid carrier may be water or DMSO (dimethylsulphoxide).

Suitable solid carriers include, for example, talc, titanium dioxide, pyrophyllite clay, silica, attapulgite clay, kieselguhr, chalk, diatomaceous earth, lime, calcium carbonate, bentonite clay, fuller's earth, cotton seed hulls, wheat flour, soybean flour, pumice, wood flour, walnut shell flour and lignin.

An adjuvant may be a surface-active agent, crystallisation inhibitor, viscosity modifier, suspending agents, spray droplet modifiers, pigments, antioxidants, foaming agents, anti-foaming agents, light-blocking agents, compatibilizing agents, sequestering agents, neutralising agents and buffers, corrosion inhibitors, dyes, odorants, spreading agents, penetration aids, micronutrients, emollients, lubricants and sticking agents.

A composition as disclosed herein preferably is an agricultural-acceptable composition.

A composition comprising a compound according to the present invention as disclosed herein typically comprises 0.5 to 95 w/w % of active ingredient such as from 1% to 90 w/w %, such as from 2 to 80 w/w %, such as from 5 to 60 w/w %. The compound according to the present invention may be the sole active ingredient in a composition as disclosed herein. In one embodiment, a composition comprising a compound of the present invention further comprises at least one additional active ingredient. An active ingredient as defined herein has fungicidal and/or insecticidal and/or herbicidal activity or has activity as plant growth regulator. A compound or a composition of the present invention may be admixed with one or more additional ingredients having pesticidal activity such as fungicides, insecticides, herbicides, bactericides, acaricides, nematicides and/or the additional ingredient comprises plant growth regulators where appropriate. Pesticidal agents are referred to herein using their common name are known, for example, from “The Pesticide Manual”, 19th Ed., British Crop Protection Council 2021.

An additional ingredient having pesticidal activity, for instance fungicidal activity may result in an unexpected synergistic activity. Accordingly, a composition comprising a lipopeptide compound according to Formula I and an additional active ingredient, for instance malonomicin, may show a synergistic effect. A synergistic effect occurs whenever the action of an active ingredient combination is greater than the sum of the actions of the individual components. The action to be expected E for a given active ingredient combination obeys the so-called COLBY formula and can be calculated as follows (COLBY, S.R. “Calculating synergistic and antagonistic responses of herbicide combination”. Weeds, Vol. 15, pages 20-22; 1967):
ppm=milligrams of active ingredient (=a.i.) per liter of spray mixture
X=% action by active ingredient A) using p ppm of active ingredient
Y=% action by active ingredient B) using q ppm of active ingredient.

According to COLBY, the expected (additive) action of active ingredients A)+B) using p+q ppm of active ingredient is:

$$E = X + Y - \frac{X \cdot Y}{100} .$$

If the action actually observed (O) is greater than the expected action (E), then the action of the combination is super-additive, i.e. there is a synergistic effect. In mathematical terms, synergism corresponds to a positive value for the difference of (O-E). In the case of purely complementary addition of activities (expected activity), said difference (O-E) is zero. A negative value of said difference (O-E) signals a loss of activity compared to the expected activity.

Besides the actual synergistic action with respect to fungicidal activity, a composition according to the invention may also have further surprising advantageous properties. Examples of such advantageous properties that may be mentioned are: more advantageous degradability; improved toxicological and/or ecotoxicological behaviour; or improved characteristics of the useful plants including: emergence, crop yields, more developed root system, tillering increase, increase in plant height, bigger leaf blade, less dead basal leaves, stronger tillers, greener leaf colour, less fertilizers needed, less seeds needed, more productive tillers, earlier flowering, early grain maturity, less plant verse (lodging), increased shoot growth, improved plant vigor, and early germination.

The additional ingredients having pesticidal activity and/or which is a plant growth regulator may be combined with a composition of the invention and used in a method of the invention and applied simultaneously or sequentially with a composition of the invention. When applied simultaneously, these further ingredients may be formulated together with the compositions of the invention or mixed in, for example, a spray tank. As an alternative to directly admixing these further ingredients having pesticidal activity, the components may be used in separate fungicidal, insecticidal or herbicidal applications as part of a programme of fungal, insect or herbal control spread over part or all of a growing season.

The at least one additional ingredient having pesticidal activity and/or which is a plant growth regulator may be any suitable known fungicide, insecticide, herbicide and/or plant growth regulator. The at least one additional ingredient having pesticidal activity and/or plant growth regulator may be from chemical origin or biological origin, for instance from plant or microbial origin. The at least one additional ingredient having pesticidal activity in a composition as disclosed herein may be produced by a microorganism able to produce a compound according to Formula (I) according to the present invention as disclosed herein above.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compound and/or composition according to the present invention may induce resistance of a plant by a priming mechanism. Priming is a mechanism which leads to a physiological state that enables plants to respond more rapidly and/or more robustly after exposure to biotic or abiotic stress as described for instance in review article: P. Aranega-Bou et. al. Priming of plant resistance by natural compounds. Hexanoic acid as a model. Front. Plant. Sci. 1, October 2014.

In one embodiment the composition according to the present invention further comprises cyclothiazomycin C, streptimidone and/or malonomicin.

Cyclothiazomycin C is a known compound and the structure of cyclothazomycin C is disclosed on p. 3 of WO2015191789 and can be produced as disclosed in Example 4 of WO2015/191789. Malonomicin (sometimes spelt ‘malonomycin’) is {[(2S)-2-amino-3-hydroxypropanoyl]amino}{2-[(5S)-5-(aminomethyl)-4-hydroxy-2-oxo-2,5-dihydro-1H-pyrrol-3-yl]-2-oxoethyl}malonic acid of Formula II

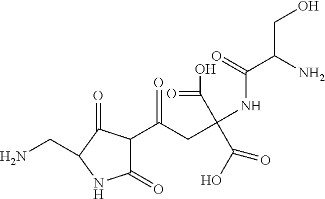 [SMILES: NCC1NC(=O)C(C(=O)CC(NC(=O)C(N)CO)(C(=O)O)C(=O)O)C1=O]

Malonomicin can be produced as disclosed in Example 1 of WO2006/078939. Malonomicin may also be prepared according to the method disclosed in Example I A and B in EP 1860939, or according to Law et al, 2018 (Nature Catalys is |VOL 1|DECEMBER 2018|977-984).

Streptimidone is a known compound of Formula III

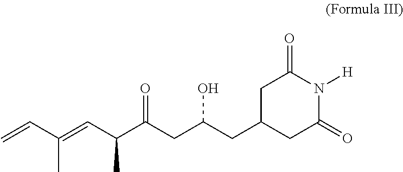 [SMILES: C=C/C(C)=C/[C@H](C)C(=O)C[C@H](O)CC1CC(=O)NC(=O)C1]

Streptimidone can be synthesised following the method disclosed in Kondo, H., Oritani, T., and Kiyota, H. Synthesis and antifungal activity of the four stereoisomers of streptimidone, a glutarimide antibiotic from Streptomyces rimosus forma paromomycinus. Eur. J. Org. Chem. (20), 3459-3462 (2000).

The present invention also relates to a composition comprising a lipopeptide compound according to Formula (I), preferably according to Formula I(a), and malonomicin. It was surprisingly found that a composition comprising a lipopeptide compound according to Formula (I) and malonomicin can exhibit an unexpected synergistic fungicidal effect. A surprising synergistic fungicidal effect of a composition comprising a lipopeptide compound according to Formula (I), preferably according to Formula I(a), and malonomicin was for instance found against Zymoseptoria tritici, Fusarium culmorum, Microdochium nivale, Botrytis cinerea, Puccinia recondita and Pyricularia oryzae.

In one embodiment, the active ingredients cyclothiazomycin C, streptimidone and/or malonomicin are produced by the microorganism able to produce compound according to Formula (I) according to the present invention as defined herein above.

A composition comprising a mixture of the compound of the invention and at least one additional active ingredient is preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight. A composition comprising a mixture of a lipopeptide compound according to Formula (I) and malonomicin comprises a ratio of a lipopeptide compound according to Formula (I) and malonomicin of from 2000:1 to 1:2000, preferably from 1000:1 to 1:1000, such as from 800:1 to 1:800, such as from 600:1 to 1:600, or from 500:1 to 1:500, from 400:1 to 1:400, from 300:1 to 1:300, or from 300:1 to 1:200. The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

A composition comprising a mixture of the compound of the invention, and one or more active ingredients as described above, for instance malonomicin, can be applied, for example, in a single “ready-mix” form, in a combined spray mixture composed from separate formulations of the single active ingredient components, such as a “tank-mix”, and in a combined use of the single active ingredients when applied in a sequential manner, i.e., one after the other with a reasonably short period, such as a few hours or days.

In one aspect, the present invention relates to a process for producing the compound or the composition according to the present invention comprising cultivating a microorganism in a suitable fermentation medium under conditions that allow production of the compound. A microorganism that is fermented in a process as disclosed herein is a microorganism able to produce the compound according to the present invention as defined herein above.

A microorganism able to produce the compound according to the present invention is for instance a bacterium of the genus Streptomyces as disclosed herein above.

Cultivating a microorganism for producing the compound according to the present invention in a suitable fermentation medium is known to a person skilled in the art. The microorganism may be fermented under aerobic or anaerobic conditions. A microorganism belonging to Streptomyces sp. is typically cultivated under aerobic conditions. A suitable fermentation medium comprises nutrients, such as a suitable carbon source such as cane or sugar beet molasses, polysaccarides, flour, starch, sugar or glucose, and a suitable nitrogen source, for instance casein hydrolysate, tryptone, ammonium sulphate, ammonia, yeast extract, peptone or urea peptides, or amino acids. The process for producing a compound according to the present invention may be performed in a batch, fed-batch or continuous culture.

In one embodiment the process further comprises producing a composition comprising the compound according to the present invention as defined herein. A microorganism able to produce the compound or composition according to the present invention is defined herein above. A microorganism able to produce the compound or composition according to the present may be able to produce further active ingredients as defined herein above, for instance cyclothiazomycin C, streptimidone and/or malonomicin.

The process according to the present invention may further comprise a step of recovering the compound according to the present invention or a salt of it. The compound according to the present invention may be recovered by suitable methods known in the art, for instance via crystallization or chromatography, eg HPLC. Recovering the compound according to the present invention may further comprise a step of purifying the compound.

The process for producing a compound according to the present invention may further comprise a step of formulating the compound into a suitable formulation or composition as defined herein above.

In one further aspect, the present invention relates to a method for controlling or preventing infestation of a plant, plant propagation material and/or harvested food crops by a phytopathogenic microorganism, by treating the plant, plant propagation material and/or harvested food crops, wherein an effective amount of the compound or a composition according to the present invention, is applied to the plant, to a part thereof or a locus thereof, plant propagation material and/or harvested food crops.

Applying an effective amount of the compound or composition of the invention in a method for controlling or preventing infestation of a plant comprises applying from 0.01 g to 5 kg of the compound of the invention (active ingredient (a.i.) per hectare (ha), preferably from 0.015 g to 500 g a.i./ha, preferably from 0.020 g to 100 g a.i./ha, preferably from 0.025 g to 50 g a.i./ha, preferably from 0.030 g to 5 g a.i./ha, preferably from 0.035 g to 500 mg a.i./ha.

When the compound of invention or composition of the present invention is used for treating seed, rates of 0.0001 to 10 g of the compound of the invention per kg of seed, such as from 0.0002 to 0.1 g per kg of seed, such as from 0.0005 to 0.001 g per kg of seed are generally sufficient. Suitably, a compound or a composition of the invention is applied either preventative, meaning prior to disease development or curative, meaning after disease development.

Phytopathogenic microorganisms that are affected by the compound of the invention are fungi and fungal vectors of disease as well as phytopathogenic bacteria and viruses. Phytopathogenic microorganisms in a method according to the present invention include the following fungi and fungal vectors of disease and phytopathogenic bacteria:

Absidia corymbifera, Albugo candida, Alternaria spp. including A. solani, Aphanomyces spp, Ascochyta spp, Aspergillus spp. including A. flavus, A. fumigatus, A. nidulans, A. niger, A. terrus, Aureobasidium spp. including A. pullulans, Bacillus subtilis, Blastomyces dermatitidis, Blumeria graminis, Blumeriella jaapii, Botryosphaeria spp. including B. dothidea, B. obtusa, Botrytis spp. including B. cinerea, Bremia lactucae, Cadophora gregata, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, C. beticola, C. kikuchii, C. sojina, Cercosporidium personatum, Cladosporium spp, Clarireedia homoeocarpa, Clavibacter spp, Claviceps purpurea, Coccidioides immitis, Cochliobolus spp, Colletotrichum spp. including C. dematium, C. lindemuthianum, C. musae, C. orbiculare, C.truncatum, Corynespora cassiicola, Cryptococcus neoformans, Diaporthe spp, Dickeya zeae, Didymella spp, Drechslera spp, Elsinoe spp, Epidermophyton spp, Eremothecium gossypiim, Erwinia spp. including E. amylovora, E. carotovora, Erysiphe spp. including E. cichoracearum, E. necator, Eutypa lata, Fusarium spp. including F. culmorum, F. graminearum, F. langsethiae, F. moniliforme, F. oxysporum, F. poae, F. proliferatum, F. pseudograminearum, F. sacchari, F. sambucinum, F. subglutinans, F. solani, F. sporotrichioides, F. tricinctum, F. virguliforme, Gaeumannomyces graminis, Gibberella spp. including G. avenacea, G. fujikuroi, G. intricans, G. moniliformis, G. zeae, Gloeodes pomigena, Gloeosporium musarum, Glomerella cingulate, Golovinomyces cichoracearum, Gymnosporangium juniperi-virginianae, Guignardia bidwellii, Gymnosporangium juniperi-virginianae, Helminthosporium spp, Hemileia spp, Histoplasma spp. including H. capsulatum, Hyaloperonospora parasitica, Kabatiella zeae, Laetisaria fuciformis, Leptographium lundbergii, Leveillula taurica, Lophodermium seditiosum, Microdochium majus, Microdochium nivale, Microsporum spp, Monilinia spp. including M. fructicola, Monographella spp including M. nivalis, Mucor spp, Mycosphaerella spp. including M. arachidis, M. fijiensis, M. graminicola, M. pomi, Nakataea oryzae, Neopseudocercosporella spp, Oculimacula spp, Oncobasidium theobromaeon, Ophiostoma spp, Pantoea stewartia, Paracoccidioides spp, Parastagonospora nodorum, Pectobacterium spp, Penicillium spp. including P. digitatum, P. italicum, Petriellidium spp, Peronosclerospora spp. including P. maydis, P. philippinensis and P. sorghi, Peronospora spp including P. destructor, Phaeosphaeria nodorum, Phakopsora pachyrhizi, Phellinus igniarus, Phialophora spp, Phlyctema vagabunda, Phoma spp, Phomopsis viticola, Phyllachora pomigena, Phyllosticta spp, Physoderma maydis, Phytophthora spp. including P. capsica, P. infestans, Plasmodiophora brassicae, Plasmopara spp. including P. halstedii, P. viticola, Plenodomus spp, Pleospora spp., Podosphaera spp. including P. leucotricha, Polymyxa graminis, Polymyxa betae, Pseudocercospora fijiensis, Pseudocercosporella herpotrichoides, Pseudomonas spp. including P. syringae, Pseudoperonospora spp. including P. cubensis, P. humuli, Pseudopeziza tracheiphila, Pseudopyrenochaeta lycopersici, Puccinia spp. including P. hordei, P. recondita, P. striiformis, P. triticina, Pyrenopeziza spp, Pyrenophora spp, Pyricularia spp. including P. oryzae, Pythium spp. including P. ultimum, Ralstonia solanacearum, Ramularia spp, Rathayibacter spp, Remotididymella destructiva, Rhizoctonia spp, Rhizomucor pusillus, Rhizopus arrhizus, Rhynchosporium spp, Robbsia andropogonis, Sarocladium oryzae, Scedosporium spp. including S. apiospermum and S. prolificans, Schizothyrium pomi, Sclerophthora macrospora, Sclerotinia spp. including S. sclerotiorum, Sclerotium spp, Septoria spp, including S. nodorum, S. tritici, Setosphaeria turcica, Sphaerotheca macularis, Sphaerotheca fusca (Sphaerotheca fuliginea), Spiroplasma kunkelii, Sporothorix spp, Stagonospora nodorum, Stagonosporopsis cucurbitacearum, Stemphylium spp, Stenocarpella macrospora, Stereum hirsutum, Streptomyces spp, Thanatephorus cucumeris, Thielaviopsis basicola, Tilletia spp, Tranzschelia discolor, Trichoderma spp. including T. harzianum, T. pseudokoningii, T. viride, Trichophyton spp, Typhula spp, Uncinula necator, Urocystis spp, Uromyces spp, Ustilago spp, Venturia spp. including V. inaequalis, Verticillium spp, Wilsonomyces carpophilus, or Xanthomonas spp, including X. oryzae and X. campestris, Xylella spp, Zymoseptoria tritici.

Phytopathogenic microorganisms that are found to be surprisingly affected by the compound and, or composition according to the present invention, are fungi, for instance fungi belonging to Blumeria, Botrytis sp, Cercospora sp. Fusarium sp. Glomerella, Microdochium Mycosphaerella, Zymoseptoria sp., Parastagonospora sp., Puccinia sp., Phaeosphaeria sp. Pyrenophora, Pyricularia sp, Sclerotinia sp. Zymoseptoria preferably the fungi belong to Blumeria graminis f.sp. tritici, Botrytis cinerea, Cercospora arachidicola, Fusarium culmorum, Glomerella lagenarium, Microdochium nivale Mycosphaerella arachidis, Parastagonospora nodorum, Puccinia recondite, Puccinia recondita f. sp. tritici, Phaeosphaeria nodorum, Pyrenophora

Phytopathogenic microorganisms that are found to be surprisingly affected by the compound or and/or composition according to the present invention, are fungi, for instance fungi belonging to Botrytis sp., Glomerella sp., Mycosphaerella sp., Puccinia sp., Phaeosphaeria sp. Pyrenophora sp, or Zymoseptoria sp. preferably fungi belonging to Botrytis cinerea, Glomerella lagenarium, Mycosphaerella arachidis, Puccinia recondita f. sp. tritici, Phaeosphaeria nodorum, Pyrenophora teres or Zymoseptoria tritici.

Controlling or preventing means reducing infestation by phytopathogenic microorganisms especially fungi, to such a level that an improvement is demonstrated.

A preferred method of controlling or preventing an infestation of crop plants by phytopathogenic microorganisms, especially fungi, or insects comprises the application of the compound or composition according to the present invention is foliar application. The frequency of application and the rate of application will depend on the risk of infestation by the corresponding pathogen or insect. However, the compound or composition according to the present invention can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compound or composition according to the present invention may also be applied to seeds (coating) by impregnating the seeds or tubers either with a liquid formulation of the fungicide or coating them with a solid formulation.

It is also possible to use the compound or composition according to the present invention as dressing agent for the treatment of plant propagation material, e.g., seed, such as fruits, tubers or grains, or plant cuttings, for the protection against fungal infections as well as against phytopathogenic fungi occurring in the soil. The propagation material can be treated with a compound and/or a composition according to the present invention before planting: seed, for example, can be dressed before being sown. The compound and/or composition according to the present invention can also be applied to grains (coating), either by impregnating the seeds in a liquid formulation or by coating them with a solid formulation. The composition can also be applied to the planting site when the propagation material is being planted, for example, to the seed furrow during sowing. Disclosed herein are such methods of treating plant propagation material and the plant propagation material so treated.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits. Germinated plants and young plants which are to be transplanted after germination or after emergence from the soil, may also be mentioned. These young plants can be protected before transplantation by a total or partial treatment by immersion.

The term “plant propagation material” is understood to denote generative parts of the plant, such as seeds, which can be used for the multiplication of the latter, and vegetative material, such as cuttings or tubers, (for example potatoes), roots, fruits, bulbs, rhizomes or parts of plants.

The term plants involve “useful plants” or “crops”. The wording “Useful plants” and “crops” are used interchangeably herein. “Useful plants” and “crops” comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes. The term “plants” also includes wood crops, such as pine trees, or woody plants.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Any suitable plant, plant propagation material or food crop may be treated in a method according according to the present invention as defined herein. Preferably the plant, plant propagation material or food crop comprises or is potato, tomato, grape, canola/oilseed rape/colza, cucurbits, groundnut, wheat, barley, corn, rice banana soya, preferably the plant is wheat or barley.

In another aspect the invention relates to the use of a compound or a composition according to the present invention as a pesticide, preferably as a fungicide, and/or as a priming agent. The features related to the compound and composition according to the present invention are as disclosed herein above. Accordingly, the present invention relates to a method for using a compound and/or composition according to the present invention as a fungicide.

### FIGURES

FIG. 1. Spectrum of light absorption (UV-VIS) 200-400 nm of a compound according to Formula I(a), Formula I(b) or Lipopetin

FIG. 2. LC-ESI-MS/MS spectrum of precursor 1204.6 m/z (M+H)⁺ for Formula I(a) depicting fragment peaks consistent with amino acids: aspartic acid, hydroxy-glutamine, serine, methyl-asparagine, methyl-phenylalanine

FIG. 3. LC-ESI-MS/MS/MS spectrum of precursor 294.2 m/z for Formula I(a) depicting peaks consistent with the molecule C14H25-OH2-C4H5ON

FIG. 4. The upfield region of the 1D ¹H NMR spectrum of a compounds according to Formula I(a) in CD₃OD at 600 MHz

FIG. 5. The downfield region of the 1D ¹H NMR spectrum of a compound according to Formula I(a) in CD₃OD at 600 MHz

FIG. 6. Graphical presentation of the lipopeptide gene cluster

### EXAMPLES

### Example 1. Source and Extraction of Compound of the Present Invention

### 1.1. Fermentation of Streptomyces sp.

Streptomyces species were ordered from culture collections disclosed in Table 1. Streptomyces sp. Saigon413 was isolated in Vietnam before 1961. Streptomyces sp. Saigon413 was deposited at the Westerdijk institute under accession number CBS149411. The deposit was made by Syngenta Ltd., Jealott's Hill Research International Centre, Bracknell, Berkshire, RG42 6EY, UK under the terms of the Budapest Treaty on the International Recognition of the Deposit of Microorganisms for the Purposes of Patent Procedure.

Streptomyces species were cultivated in Erlenmeyer flasks with a liquid medium consisting of (g/I) casein hydrolysate 10, glucose 40, K2HPO4 1.25, soytone 2, tryptone, 8 and incubated at 28 °C in an incubator shaking 150 rpm with 25 mm throw for 4 days.

The presence of the compound according to Formula I, such as a compound according to Formula I(a) and Formula I(b), in the fermentation broth was determined by isolating and purifying according to the method described in section 1.5 and identified as disclosed in section 2.

The results in Table 1 show that a compound according to Formula I(a) was present in the fermentation broth of various Streptomyces species.

1.2. Isolation of 16S rDNA, Whole Genome Sequencing and Species Identification

Genomic DNA was isolated from Streptomyces sp. Saigon413 using the method described in Kutchma et al. (1998) Biotechniques 24 (3): 452-457. The 16S rRNA gene was amplified using universal 16S primers and sequenced using Sanger sequencing. The 16S rRNA of Streptomyces sp. Saigon413 is shown in SEQ ID NO: 1.

The species of strain Streptomyces sp. Saigon413 was identified by comparing the 16 S rRNA sequence according to SEQ ID NO:1 with publicly available 16S rRNA sequences that were extracted using whole genome sequence assembly of genomes from Streptomyces species (based on The Genome Taxonomy Database GTDB (Parks, D. H., et al. (2021). GTDB: Nucleic Acids Research, 50: D785-D794) using barrnap v0.9. Based on this comparative analysis Streptomyces sp. Saigon413 was identified as a Streptomyces chrestomyceticus species. The sequence identity between the 16S rRNA sequence of Streptomyces sp. Saigon413 and the publicly available S. chrestomyceticus NRRL-3672 was 99.87%, which was determined using Muscle v3.8.31 and R package Seqinr v4.2-16.

In addition, whole genome sequencing, using the genomic DNA from Streptomyces sp. Saigon413, was completed using both Pacific Biosciences and Illumina sequencing technologies. The genome was assembled using HFAP4 and polished with Pilon using the Illumina reads. Genomic DNA was also extracted from Streptomyces rimosus CBS 492.64, Streptomyces rimosus CBS 570.66, Streptomyces rimosus CBS 569.66, Streptomyces chrestomyceticus DSM 41224, Streptomyces rimosus subsp. rimosus DSM 40673, and Streptomyces rimosus subsp. rimosus DSM 41057 using a method described in Kieser et. al., (2000) Practical Streptomyces Genetics. Whole genome sequencing for these strains was completed using Nanopore Sequencing technology and the genomes were assembled with Flye (Kolmogorov, M., et. al., (2019), Nature Biotechnology, 37, 540).

Following assembly of the genome from Streptomyces sp. Saigon 413 and publicly available genomes, the average nucleotide identity (ANI) was calculated between Streptomyces sp. Saigon413 and closely related Streptomyces strains using fastANI (Jain, C., et. al. (2018), Nature Communications, 9, 5114) (Table 1). The highest percentage identity (ANI) of the genome of Streptomyces sp. Saigon413 was 96.9% with the publicly available genome of S. chrestomyceticus NRRL B-3672.

Using the 16SRNA sequence identity and ANI score (%), it was also found that the strains CBS 596.66, CBS570.66 and DSM 41429 were Streptomyce chrestomyceticus strains and not a Streptomyces rimosis or Streptomyces paromomycinus strain as indicated by the depository institute. In Table 1, the percentage identity of the whole genome and the 16SRNA sequence of several Streptomyces species to the one of Streptomyces sp. Saigon 413 is shown.

TABLE 1
Identification of lipopeptide compound of Formula I(a) in different Streptomyces species

| | | | | | 16SRNA | Compound |
|---|---|---|---|---|---|---|
| | Species as | | ANI (%) | ANI (%) | sequence | of |
| strain ID | indicated by | Species indicated | vs. NRRL | vs. | identity | Formula |
| (Collection) | the source | by ANI | B3672 | CBS149411 | (%) | I(a) |
| CBS 149411 | Streptomyces sp. | Streptomyces | 96.90 | 100 | 100 | yes |
| (CBS) | Saigon413 | chrestomyceticus | | | | |
| CBS 492.64 | S. rimosus | Streptomyces sp. | 90.02 | 90.04 | 99.48 | yes |
| (CBS) | | | | | | |
| CBS 569.66 | S. rimosus | Streptomyces | 96.63 | 99.74 | 99.94 | yes |
| (CBS) | | chrestomyceticus | | | | |
| CBS 570.66 | S. rimosus | Streptomyces | 96.65 | 99.74 | 99.94 | yes |
| (CBS) | | chrestomyceticus | | | | |
| CBS 612.68 | S. albofaciens | Streptomyces | 89.91 | 89.92 | 98.82 | No |
| (CBS) | | albofaciens | | | | |
| CBS 938.68 | S. rimosus subsp. | Streptomyces | 89.88 | 89.81 | 98.89 | No |
| (CBS) | rimosus | rimosus | | | | |
| DSM 40673 | S. rimosus | Streptomyces | 89.86 | 89.78 | 99.02 | No |
| (DSMZ) | | rimosus | | | | |
| DSM 41057 | S. rimosus | Streptomyces | 90.00 | 89.96 | 98.89 | No |
| (DSMZ) | | rimosus | | | | |
| DSM 41224 | S. chrestomyceticus | Streptomyces | 99.97 | 96.96 | 99.87 | No |
| (DSMZ) | | chrestomyceticus | | | | |
| DSM 41429 | S. paromomycinus | Streptomyces | 95.88 | 95.95 | 99.87 | yes |
| (DSMZ) | | chrestomyceticus | | | | |
| DSM-41561 | S. rimosus subsp. | Streptomyces | 89.79 | 89.84 | 99.017 | No |
| (DSMZ) | rimosus | rimosus | | | | |
| NRRL B- | S. rimosus | Streptomyces | 89.82 | 89.75 | 98.89 | No |
| 2626 (ARS) | | rimosus | | | | |
| NRRL B- | S. chrestomyceticus | Streptomyces | 100 | 96.9 | 99.87 | yes |
| 3672 (ARS) | | chrestomyceticus | | | | |
| NRRL B- | S. rimosus | n.d | | n.d. | n.d. | No |
| 3698 (ARS) | | | | | | |
| NRRL WC- | S. rimosus | Streptomyces | | 89.87 | 98.95 | No |
| 3869 (ARS) | | rimosus | | | | |

CBS Westerdijk fungal diversity institute: Uppsalalaan 8, 3584 CT, Utrecht, The Netherlands
DSMZ German Collection of Microorganisms and Cell Cultures: Inhoffenstraße 7B, 38124 Braunschweig, Germany.
ARS ARS Culture Collection (NRRL), 1815 N. University Street, Peoria, IL 61604, USA

1.3. Identification of the Biosynthetic Gene Cluster Producing a Lipopeptide According to Formula I in Streptomyces sp. Saigon413

To identify genes involved in the production of the lipopeptide according to Formula I, the assembled genome (see Example 1.2) was run through AntiSMASH (version 5.1.1, Blin et. al., Nucleic Acids Res (2019) doi: 10.1093/nar/gkz310), a commonly used tool to assist with the identification of biosynthetic gene clusters responsible for the production of secondary metabolites.

With the identification of lipopeptide compound being a lipopeptide family (see 1 above, and the AntiSMASH output we were able to deduce that the lipopeptide compound is produced by a non-ribosomal peptide synthetase (NRPS gene cluster). The identification of a NRPS gene cluster responsible for the biosynthesis of the lipoeptide compound was based upon the structural analysis of the compound and the amino acids that are incorporated into the depsipeptide core of the lipopeptide compound. Within Streptomyces sp. Saigon413, only one NRPS biosynthetic gene cluster (FIG. 11) enabling the incorporation of amino acid precursors including asparagine, aspartic acid, glutamic acid, phenylalanine, serine and threonine, was identified and therefore was associated with the production of the lipopeptide compounds of Formula I(a) and (b).

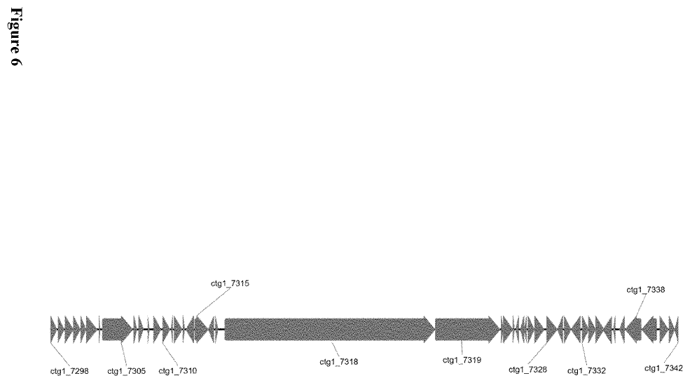

The NRPS biosynthetic gene cluster contains 45 coding sequences including two coding sequences for NRPS genes, a coding sequence for a regulator, and coding sequences responsible for the biosynthesis of a precursor incorporated into lipopeptide of Formula I (FIG. 6 and Table 4).

TABLE 4
Coding sequences present in NRPS biosynthetic gene cluster responsible for
the production of the lipopeptide according to Formula I. Annotations provided
are based upon pBLAST search using the non-redundant protein sequences
on the National Centre for Biotechnology Information database.

| | SEQ ID | | |
|---|---|---|---|
| Gene ID | DNA | Translated Protein | Annotation |
| CDS_1 (ctg1_7298) | SEQ ID NO: 2 | SEQ ID NO: 47 | O-methyltransferase |
| CDS_2 (ctg1_7299) | SEQ ID NO: 3 | SEQ ID NO: 48 | Oxygenase |
| CDS_3 (ctg1_7300) | SEQ ID NO: 4 | SEQ ID NO: 49 | Hypothetical protein |
| CDS_4 (ctg1_7301) | SEQ ID NO: 5 | SEQ ID NO: 50 | Deoxynucleoside kinase |
| CDS_5 (ctg1_7302) | SEQ ID NO: 6 | SEQ ID NO: 51 | Chloramphenicol |
| | | | phosphotransferase family |
| | | | protein |
| CDS_6 (ctg1_7303) | SEQ ID NO: 7 | SEQ ID NO: 52 | MFS transporter |
| CDS_7 (ctg1_7304) | SEQ ID NO: 8 | SEQ ID NO: 53 | Unknown protein |
| CDS_8 (ctg1_7305) | SEQ ID NO: 9 | SEQ ID NO: 54 | ALF repeat-containing protein |
| CDS_9 (ctg1_7306) | SEQ ID NO: 10 | SEQ ID NO: 55 | Hypothetical protein |
| CDS_10 (ctg1_7307) | SEQ ID NO: 11 | SEQ ID NO: 56 | Hypothetical protein |
| CDS_11 (ctg1_7308) | SEQ ID NO: 12 | SEQ ID NO: 57 | Hypothetical protein |
| CDS_12 (ctg1_7309) | SEQ ID NO: 13 | SEQ ID NO: 58 | Radical SAM enzyme |
| CDS_13 (ctg1_7310) | SEQ ID NO: 14 | SEQ ID NO: 59 | Phytanoyl-CoA dioxygenase |
| CDS_14 (ctg1_7311) | SEQ ID NO: 15 | SEQ ID NO: 60 | Transcriptional regulator |
| CDS_15 (ctg1_7312) | SEQ ID NO: 16 | SEQ ID NO: 61 | NADP-dependent |
| | | | oxidoreductase |
| CDS_16 (ctg1_7313) | SEQ ID NO: 17 | SEQ ID NO: 62 | Short-chain |
| | | | dehydrogenase/reductase |
| CDS_17 (ctg1_7314) | SEQ ID NO: 18 | SEQ ID NO: 63 | Transcriptional regulator |
| CDS_18 (ctg1_7315) | SEQ ID NO: 19 | SEQ ID NO: 64 | Drug resistance transporter |
| CDS_19 (ctg1_7316) | SEQ ID NO: 20 | SEQ ID NO: 65 | Transcriptional regulator (TetR |
| | | | family) |
| CDS_20 (ctg1_7317) | SEQ ID NO: 21 | SEQ ID NO: 66 | Hypothetical protein |
| CDS_21 (ctg1_7318) | SEQ ID NO: 22 | SEQ ID NO: 67 | Non-ribosomal peptide |
| | | | synthetase |
| CDS_22 (ctg1_7319) | SEQ ID NO: 23 | SEQ ID NO: 68 | Non-ribosomal peptide |
| | | | synthetase |
| CDS_23 (ctg1_7320) | SEQ ID NO: 24 | SEQ ID NO: 69 | MbtH-like protein |
| CDS_24 (ctg1_7321) | SEQ ID NO: 25 | SEQ ID NO: 70 | TauD/TfdA family dioxygenase |
| CDS_25 (ctg1_7322) | SEQ ID NO: 26 | SEQ ID NO: 71 | SAM-dependent |
| | | | methyltransferase |
| CDS_26 (ctg1_7323) | SEQ ID NO: 27 | SEQ ID NO: 72 | Hypothetical protein |
| CDS_27 (ctg1_7324) | SEQ ID NO: 28 | SEQ ID NO: 73 | VOC family protein |
| CDS_28 (ctg1_7325) | SEQ ID NO: 29 | SEQ ID NO: 74 | Transcriptional regulator (ArsR |
| | | | family) |
| CDS_29 (ctg1_7326) | SEQ ID NO: 30 | SEQ ID NO: 75 | Domain of unknown function |
| | | | (DUF) |
| CDS_30 (ctg1_7327) | SEQ ID NO: 31 | SEQ ID NO: 76 | Transcriptional regulator (AraC |
| | | | family) |
| CDS_31 (ctg1_7328) | SEQ ID NO: 32 | SEQ ID NO: 77 | Transporter |
| CDS_32 (ctg1_7329) | SEQ ID NO: 33 | SEQ ID NO: 78 | Domain of unknown function |
| | | | (DUF) |
| CDS_33 (ctg1_7330) | SEQ ID NO: 34 | SEQ ID NO: 79 | Aminoglycoside |
| | | | adenylyltransferase |
| CDS_34 (ctg1_7331) | SEQ ID NO: 35 | SEQ ID NO: 80 | FxbS family cyclophane- |
| | | | forming radical SAM/SPASM |
| | | | peptide maturase |
| CDS_35 (ctg1_7332) | SEQ ID NO: 36 | SEQ ID NO: 81 | 2′-5′ RNA ligase family protein |
| CDS_36 (ctg1_7333) | SEQ ID NO: 37 | SEQ ID NO: 82 | Transcriptional regulator (MerR |
| | | | family) |
| CDS_37 (ctg1_7334) | SEQ ID NO: 38 | SEQ ID NO: 83 | Hypothetical protein |
| CDS_38 (ctg1_7335) | SEQ ID NO: 39 | SEQ ID NO: 84 | Chorophyllase |
| CDS_39 (ctg1_7336) | SEQ ID NO: 40 | SEQ ID NO: 85 | Hypothetical protein |
| CDS_40 (ctg1_7337) | SEQ ID NO: 41 | SEQ ID NO: 86 | ABATE domain-containing |
| | | | protein (putative stress- |
| | | | induced transcription |
| | | | regulator) |
| CDS_41 (ctg1_7338) | SEQ ID NO: 42 | SEQ ID NO: 87 | ABC transporter (ATP-binding |
| | | | cassette protein) |
| CDS_42 (ctg1_7339) | SEQ ID NO: 43 | SEQ ID NO: 88 | ABC transporter (ATP-binding |
| | | | cassette protein) |
| CDS_43 (ctg1_7340) | SEQ ID NO: 44 | SEQ ID NO: 89 | Quinone oxidoreductase |
| CDS_44 (ctg1_7341) | SEQ ID NO: 45 | SEQ ID NO: 90 | Transcriptional regulator (TetR |
| | | | family) |
| CDS_45 (ctg1_7342) | SEQ ID NO: 46 | SEQ ID NO: 91 | Helix-turn-helix domain |
| | | | containing protein |

1.4. Deletion of Genomic Region Including CDS_21 (SEQ ID NO: 22) and CDS_22 (SEQ ID NO: 23) from Streptomyces sp. Saigon413 and Phenotypic Analysis

To confirm the identified biosynthetic gene cluster was associated with the production of [insert compound ID here], a region containing two non-ribosomal peptide synthetase genes encoded by ctg_7318 and ctg_7319 (SEQ ID NO: 22 and 23) was deleted from Streptomyces sp. Saigon413. To generate Streptomyces sp. Saigon41347318-7319, plasmid pBCon2192 was used. Plasmid pBCCon2192 was prepared from pRAR017 and contained regions of homology to either side of the region to be deleted from the strain (facilitating primary and secondary crossovers).

Plasmid pBCon2192 was used to transform E. coli ET12567/pUZ8002 using a standard electroporation method, and then introduced into Streptomyces sp. Saigon413 by mycelial conjugation (T. Kieser et. al., Practical Streptomyces Genetics, 2000, John Innes Foundation, Norwich). Thiostrepton resistant colonies were patched on ISP-4 agar media supplemented with 40 μg/ml thiostrepton and 25 μg/ml nalidixic acid. These patches were initially incubated for 6 days at 28 °C allowing plasmid replication. After 6 days at 28 °C, strains were re-patched on ISP-4 agar media supplemented with 40 μg/ml thiostrepton and incubated at 37 °C for further 6 days to force primary integration. After 6 days at 37 °C, the obtained strains were transferred onto ISP-4 solid agar media without selection and incubated for 15 days at 28 °C to allow a second crossover.

After 15 days of growth, strains were collected in 20% glycerol. 100 μl of the cell suspension was used to inoculate fresh plates, as well as to make serial dilutions up to 10⁻¹⁰. 100 μl of 10⁻⁸ to 10⁻¹⁰ were then plated onto ISP-4 agar plate. Plates were incubated at 28 °C until single colonies were observed.

Single colonies were double patched on non-selective and thiostrepton selective ISP-4 agar media. Sensitive patches (representing secondary recombination) were then screened via PCR using gDNA isolated with FastSpin kit for soil (MP Biomedicals) to identify correct colonies.

To confirm the region containing both SEQ ID NO: 22 and SEQ ID NO: 22 has been removed from the strain, a primer pair binding to the outside of the deleted region was used. Sanger sequencing of the PCR product and alignment to the Streptomyces sp. Saigon413 genome, confirmed deletion of the genomic region containing SEQ ID NO: 22 and SEQ ID NO:23. Additionally, full genome analysis using Illumina-PCR-free sequencing confirmed that no other alterations had been made to the genome.

Cultivation of Streptomyces sp. Saigon413Δ7318-7319 and analysis of extracts from the strain confirmed that the lipopeptide compound according to Formula I(a) was no longer produced by the strain, confirming SEQ ID NO: 22 and SEQ ID NO: 23 are essential for production of the lipopeptide compound according to Formula I(a) 1.5. Purification of the compound according to the present invention

The mycelia from fermentation broth from Streptomyces strains disclosed in Table 1 were separated via centrifugation and the supernatant was treated with butanol. The butanol was removed and the extract partitioned between water and ethyl acetate. The lipopeptides were purified from the ethyl acetate fraction by preparative reverse phase (C18) HPLC. The lipopeptides were relatively apolar and elute in the higher organic fraction in a gradient system with 0.1% formic acid and acetonitrile (0.1% formic acid). A gradient of 60% Aqueous to 40% Aqueous with the above solvents allowed separation of a compound according to Formula I(a) and Formula I(b).

Similarly, the fermentation broth of Streptomyces sp. Saigon413 was processed.

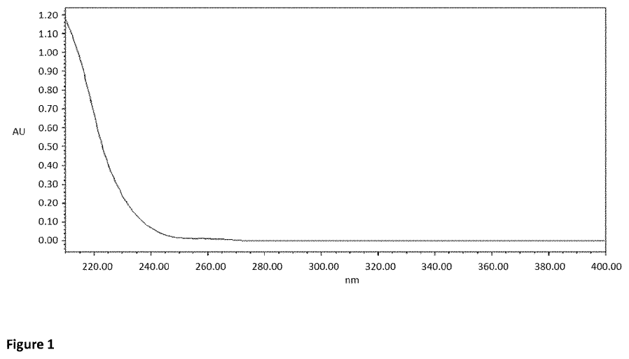

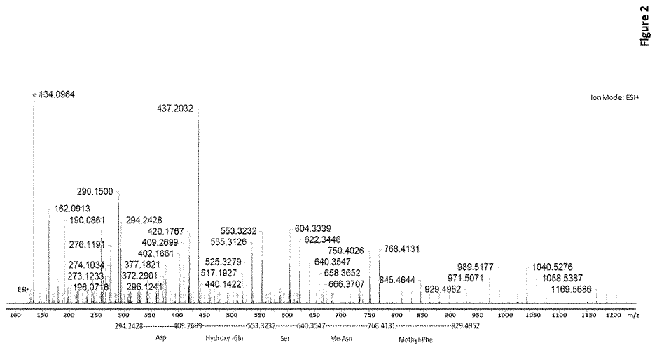

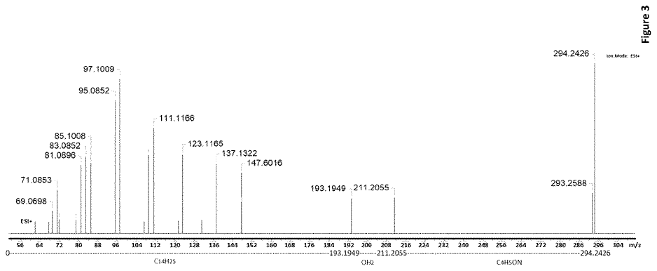

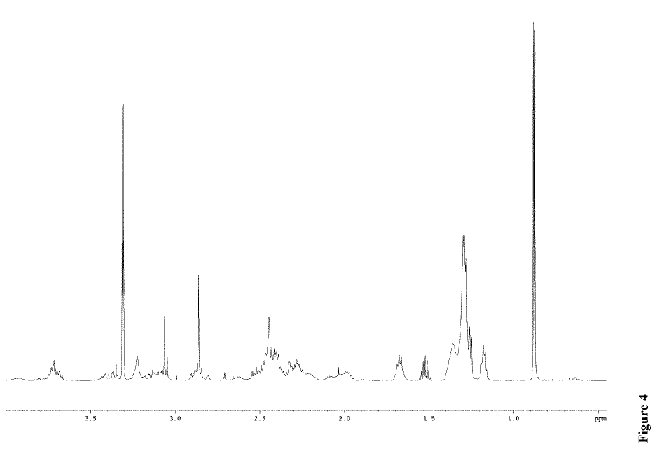

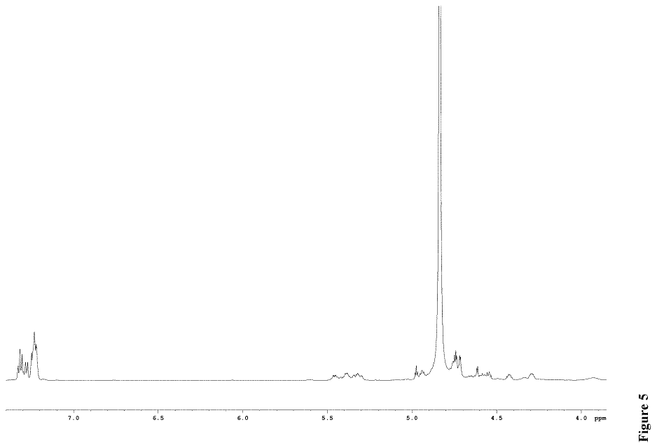

A stock solution for the compound of Formula I(a) and Formula I(b) was produced in DMSO (max. 10 mg/ml), which was further diluted with water plus 0.025% Tween20 to produce suitable working concentrations for biological efficacy assays. Compounds were detected by UV-VIS (FIG. 1), mass spectrometry (FIGS. 2 and 3) and NMR spectrometry (FIGS. 4 and 5).

### 1.6. Lipopeptin A

Lipopeptin A was purchased from Fundación MEDINA, Centro de Excelencia en Investigación de Medicamentos Innovadores en Andalucía, Avda. del Conocimiento 34, Edificio Centro de Desarrollo Farmacéutico y Alimentario, Parque Tecnológico de Ciencias de la Salud, 18016 Granada (ESPAÑA).

### Example 2. Characterisation of the Compound of Formula I

### 2.1 Liquid Chromatography and High-Resolution Mass Spectrometry

Spectra were recorded on an Orbitrap ID-X Tribrid Mass Spectrometer from Thermo Scientific equipped with an OptaMax NG Heated Electrospray Source (Spray Voltage: Static, Polarity Ion (V): 3400 (Positive ion mode) & 2400 (Negative ion mode), Sheath Gas (Arb): 40, Aux Gas (Arb): 5, Sweep Gas (Arb): 1, Ion Transfer Tube Temperature: 350 °C, Vaporizer Temperature: 350 °C). The Scan Parameters were as follows;

Experiment 1: MS OT (Orbitrap Resolution: 50,000, Scan Range (m/z): 200 to 2000, RF Lens (%): 60, AGC Target: Standard, Maximum Injection Time Mode: Auto, Microscans: 1, Data Type: Profile, Polarity: Both),

Experiment 2: MS2 OT CID (MSn Level (n): 2, Isolation Window (m/z): 1.0, Activation Type: CID, CID Collision Energy (%): 30, Detector Type: Orbitrap, Orbitrap Resolution: 30,000, RF Lens (%): 60, Polarity: Positive), Experiment 3: tMS2 OT HCD (MSn Level (n): 2, Isolation Window (m/z): 1.0, Activation Type: HCD, HCD Collision Energy (%): 30, Detector Type: Orbitrap, Orbitrap Resolution: 30,000, RF Lens (%): 60, Polarity: Positive), Experiment 4: tMS3 OT HCD (MSn Level (n): 3, Isolation Window (m/z): 1.6, Activation Type: HCD, HCD Collision Energy (%): 30, MS2 Isolation Window (m/z): 2, MS2 Activation Type: HCD, MS2 HCD Collision Energy (%): 30, Detector Type: Orbitrap, Orbitrap Resolution: 30,000, RF Lens (%): 60, Polarity: Positive). The mass spectrometer was connected to a Vanquish Flex UHPLC from Thermo Scientific using a Vanquish Split Sampler FT, Vanquish Binary Pump F, Vanquish Column Compartment H, Vanquish Diode Array Detector FG and Vanquish Charged Aerosol Detector.

Liquid Chromatography Conditions included: Waters ACQUITY UPLC C18 column 1.7 μm 3.0×50 mm, P.N. 186004660. Temp: 40 °C, DAD wavelength range: 250 to 260 nm, Solvent gradient: Solvent A: H2O with 0.1% formic acid, Solvent B: CH3CN with 0.1% formic acid, gradient: 0 min 10% B, 90% A; 4.00 min 90% B, 10% A; 4.25 min 90% B, 10% A; 4.50 min 10% B, 90% A; 5.00 min 10% B, 90% A, Flow rate: 1.0 ml/min, Injection volume: 2 μL, Total run time: 5.0 min. A purified fermentation broth as described above was injected.

FIGS. 2 and 3 show LC-ESI-MS/MS/MS spectra of a compound of Formula I(a).

A compound according to Formula I(a) and Formula I(b) was identified in the fermentation broth of Streptomyces sp. Saigon413 deposited under accession number CBS 149411.

### 2.2. NMR Spectroscopy

NMR spectra were recorded on a Bruker AVIII 600 NMR spectrometer, equipped with a 5 mm Bruker (¹H/¹⁹F)/¹³C/¹⁵N TCI cryoprobe fitted with Z gradients, using standard Bruker pulse sequences. Samples were dissolved in CD₃OD, and the spectra were recorded at 300° K and referenced to the residual solvent signal at 3.31 ppm for ¹H. FIGS. 4 and 5 each cover half of the 1H NMR spectrum of a compound according to Formula 1(a).

### 2.3. Molecular Composition and Mass

The molecular composition and mass of a compound according to Formula I(a) and Formula I(b) was determined using the results of liquid chromatography and high-resolution mass spectrometry as disclosed in 2.1. The compounds of Formula I(a) and I(b) have the following composition.

Formula I(a) Lipopeptide 1204: Molecular composition C55H85N11O19 and exact mass of 1203.602. Formula I(b) Lipopeptide 1218: Molecular composition C56H87N11O19 and exact mass of 1217.618. The reference Lipopeptin A has the following composition C54H84N10019 and exact mass 1176.591421.

### 2.4. Solubility

The solubility of a compound of Formula I(a), Formula I(b) and Lipopeptin A was determined in water and DMSO:

| Compound | solvent (pH) | solubility (ppm) |
|---|---|---|
| Formula I (a) Lipopeptide 1204 | water (2.08) | 21.4 |
| Formula I (a) Lipopeptide 1204 | water (5.55) | >10′000 |
| Formula I (a) Lipopeptide 1204 | DMSO | >10′000 |
| Formula I (b) Lipopeptide 1218 | DMSO | >10′000 |
| Lipopeptin A | DMSO | >10′000 |

### Example 3. Activity of a Compound According to the Present Invention on Plants

### 3.1 Fungicidal Activity in Liquid Culture Assays

Mycelia fragments or conidia suspensions of a fungus, prepared either freshly from liquid cultures of the fungus or from cryogenic storage, were directly mixed into nutrient broth.

A stock solution for compound of Formula I(a) and Formula I(b) was produced in DMSO (max. 10 mg/ml), which was diluted with water plus 0.025% Tween®20 to produce a 10× concentrated sample and 10 μl of this solution was pipetted into a microtiter plate (96-well format). The nutrient broth containing the fungal spores/mycelia fragments was then added to give an end concentration of the tested compound. The test plates were incubated in the dark at 24 °C and 96% rh. The inhibition of fungal growth was determined photometrically after 2-7 days, depending on the pathosystem, and percent antifungal activity relative to the untreated check was calculated.

The effect of the test compound (Formula I(a), I(b) and Lipopeptin A) was tested against the following fungi under the conditions as outlined above and specifically herein below:

Botryotinia fuckeliana (Botrytis cinerea)/Liquid Culture (Gray Mould)

Conidia of the fungus from cryogenic storage were directly mixed into nutrient broth (Vogels broth). The inhibition of growth is determined photometrically 3-4 days after application.

Glomerella lagenarium (Colletotrichum lagenarium)/Liquid Culture (Anthracnose)

Conidia of the fungus from cryogenic storage were directly mixed into nutrient broth (PDB potato dextrose broth). The inhibition of growth was measured photometrically 3-4 days after application.

Mycosphaerella arachidis (Cercospora arachidicola)/liquid culture (early leaf spot) Conidia of the fungus from cryogenic storage were directly mixed into nutrient broth (PDB potato dextrose broth). The inhibition of growth was determined photometrically 4-5 days after application.

Zymoseptoria tritici (Septoria tritici)/liquid culture (Septoria leaf blotch) Conidia of the fungus from cryogenic storage were directly mixed into nutrient broth (PDB potato dextrose broth). The inhibition of growth was determined photometrically 4-5 days after application.

The results are shown in Table 2 and Table 3.

TABLE 2
Control of fungal development by a compound according
to Formula I(a) and I(b) in liquid culture.

| | | Botryotinia | Glomerella | Mycosphaerella | Zymoseptoria |
|---|---|---|---|---|---|
| | concentration | fuckeliana | lagenarium | arachidis | tritici |
| compound | (ppm) | % reduction fungal growth | | | |
| Formula I (a) | 100 | 50 | 100 | 100 | 100 |
| | 33 | 0 | 90 | 100 | 100 |
| | 11 | 0 | 50 | 100 | 70 |
| | 3.7 | 0 | 0 | 100 | 0 |
| | 1.2 | 0 | 0 | 0 | 0 |
| | 0.41 | 0 | 0 | 0 | 0 |
| Formula I (b) | 100 | 50 | 0 | 100 | 70 |
| | 33 | 0 | 0 | 100 | 70 |
| | 11 | 0 | 0 | 90 | 50 |
| | 3.7 | 0 | 0 | 0 | 0 |
| | 1.2 | 0 | 0 | 0 | 0 |
| | 0.41 | 0 | 0 | 0 | 0 |

TABLE 3
Control of fungal development by a compound according
to Formula I(a) and I(b) in a liquid culture.

| | | Botryotinia | Glomerella | Mycosphaerella | Zymoseptoria |
|---|---|---|---|---|---|
| | concentration | fuckeliana | lagenarium | arachidis | tritici |
| compound | (ppm) | % reduction fungal growth | | | |
| Formula I (a) | 100 | 70 | 50 | 100 | 90 |
| | 33 | 70 | 0 | 100 | 90 |
| | 11 | 50 | 0 | 70 | 70 |
| | 3.7 | 50 | 0 | 0 | 0 |
| | 1.2 | 0 | 0 | 0 | 0 |
| | 0.41 | 0 | 0 | 0 | 0 |
| Formula I (b) | 100 | 70 | 0 | 100 | 90 |
| | 33 | 50 | 0 | 100 | 90 |
| | 11 | 50 | 0 | 100 | 90 |
| | 3.7 | 0 | 0 | 0 | 0 |
| | 1.2 | 0 | 0 | 0 | 0 |
| | 0.41 | 0 | 0 | 0 | 0 |
| Lipopeptin A | 100 | 0 | 0 | 0 | 70 |
| | 33 | 0 | 0 | 0 | 0 |
| | 11 | 0 | 0 | 0 | 0 |
| | 3.7 | 0 | 0 | 0 | 0 |
| | 1.2 | 0 | 0 | 0 | 0 |
| | 0.41 | 0 | 0 | 0 | 0 |

The results in Table 2 and Table 3 show that the lipopeptide compounds of Formula I(a) and Formula I(b) were active against several phytopathogenic fungi providing up to 100% control. Compounds of Formula I(a) and Formula I(b) have similar efficacy, while Lipopeptin A was less active.

### 3.2. Leaf Disc or Leaf Segment Tests in Well Plates

Leaf disks or leaf segments of various plant species were cut from plants grown in the greenhouse. The cut leaf disks, or segments were placed in multiwell plates (24-well format) onto water agar. The leaf disks were sprayed with a test solution before inoculation with the fungal spores. Compounds to be tested were prepared as DMSO solutions (max. 10 mg/ml) which were diluted to the appropriate concentration with water plus 0.025% Tween® 20 just before spraying. The inoculated leaf disks or segments were incubated under defined conditions (temperature, relative humidity, light, etc.) according to the respective test system. A single evaluation of disease level was carried out by visual evaluation 3-9 days days after inoculation, depending on the pathosystem as indicated below. Percent disease control relative to the untreated check leaf disks or segments was then calculated.

Puccinia recondita f. Sp. Tritici/Wheat/Leaf Disc (Brown Rust)

Wheat leaf segments cv. Kanzler were placed on agar in multiwell plates (24-well format) and sprayed with the formulated test compound diluted in water. The leaf disks were inoculated with a spore suspension of the fungus 1 day after application. The inoculated leaf segments were incubated at 19 °C and 75% rh under a light regime of 12 h light/12 h darkness in a climate cabinet and the activity of a compound was assessed as percent disease control compared to untreated when an appropriate level of disease damage appears in untreated check leaf segments (7-9 days after application).

Phaeosphaeria Nodorum (Septoria nodorum)/Wheat/Leaf Disc (Glume Blotch)

Wheat leaf segments cv. Kanzler were placed on agar in a multiwell plate (24-well format) and sprayed with the formulated test compound diluted in water. The leaf disks were inoculated with a spore suspension of the fungus 2 days after application. The inoculated test leaf disks were incubated at 20 °C and 75% rh under a light regime of 12 h light/12 h darkness in a climate cabinet and the activity of a compound was assessed as percent disease control compared to untreated when an appropriate level of disease damage appears in untreated check leaf disks (5-7 days after application)

Pyrenophora teres/Barley/Leaf Disc Preventative (Net Blotch)

Barley leaf segments cv. Hasso were placed on agar in a multiwell plate (24-well format) and sprayed with the formulated test compound diluted in water. The leaf segments were inoculated with a spore suspension of the fungus 2 days after application. The inoculated leaf segments were incubated at 20 °C and 65% rh under a light regime of 12 h light/12 h darkness in a climate cabinet and the activity of a compound was assessed as disease control compared to untreated when an appropriate level of disease damage appears in untreated check leaf segments (5-7 days after application).

TABLE 4
Control of fungal development by a compound according
to Formula I(a) or (b) on a leaf disc or leaf segment

| | concentration | Puccinia recondita f. sp. tritici |
|---|---|---|
| compound | (ppm) | % reduction of fungal growth |
| Formula I (a) | 200 | 100 |
| | 67 | 90 |
| | 22 | 0 |
| | 7.4 | 0 |
| | 2.5 | 0 |
| | 0.82 | 0 |
| Formula I(b) | 200 | 70 |
| | 67 | 45 |
| | 22 | 0 |
| | 7.4 | 0 |
| | 2.5 | 0 |
| | 0.82 | 0 |

TABLE 5
Control of fungal development by a compound according to Formula
I(a) or (b) or Lipopeptin A on a leaf disc or leaf segment.

| | concen- | Puccinia recondita | Phaeosphaeria | Pyrenophora |
|---|---|---|---|---|
| | tration | f. sp. tritici | nodorum | teres |
| compound | (ppm) | % reduction of fungal growth | | |
| Formula | 200 | 95 | 50 | 80 |
| I(a) | 67 | 90 | 0 | 60 |
| | 22 | 35 | 0 | 0 |
| | 7.4 | 0 | 0 | 0 |
| | 2.5 | 0 | 0 | 0 |
| | 0.82 | 0 | 0 | 0 |
| Formula | 200 | 95 | 0 | 90 |
| I (b) | 67 | 70 | 0 | 90 |
| | 22 | 35 | 0 | 0 |
| | 7.4 | 0 | 0 | 0 |
| | 2.5 | 0 | 0 | 0 |
| | 0.82 | 0 | 0 | 0 |
| Lipopeptin | 200 | 35 | 0 | 0 |
| A | 67 | 0 | 0 | 0 |
| | 22 | 0 | 0 | 0 |
| | 7.4 | 0 | 0 | 0 |
| | 2.5 | 0 | 0 | 0 |
| | 0.82 | 0 | 0 | 0 |

The results in Table 4 and 5 show that a compound according to Formula I(a) or I(b) was active in the assays providing up to 95% reduction of fungal growth on several fungal species. Compounds of Formula I(a) and Formula I(b) have similar efficacy, while Lipopeptin A was found less active.

### 3.3. Leaf Painting Assay in Wheat Seedling

Wheat seedlings of the variety Riband (for Zymoseptoria tritici trials) or variety Arina (for Puccinia recondite trials) were grown in a glasshouse until 14 d after seeding. At this time, such seedlings usually have a first leaf fully emerged (designated L1), a second leaf fully emerged (L2) and a third leaf (L3) that was partially emerged and in the process of development. Using a permanent pen, two dots were applied on the second leaf such as to generate three segments of roughly equal size: Segment A (at the base), segment B (middle part) and segment C (upper part of the leaf). A stock solution of the test compound was produced in DMSO at a concentration of 10′000 ppm. The stock solution was then further diluted in water supplemented with Tween® 20 to a final concentration of 200 ppm or higher of test compound, 0.05% Tween® 20 and 2% DMSO. The diluted compound was applied to the middle segment of L2 using a conventional cotton stick; the cotton stick was soaked in the diluted compound and rubbed several times on the adaxial leaf surface between the two marks. 1 day later, the complete plant was inoculated with a fungal spore suspension using a paint brush and application of the spore suspension until before run-off.

For infection with Zymoseptoria tritici: The test plants were inoculated by spraying a spore suspension on them 1 day after application (1.5Mio spores per ml in water supplemented with 0.01% Tween®20). After an incubation period of 4 days at 22 °C/21 °C. (day/night) and 95% rh, the inoculated test plants were kept at 22 °C/21 °C (day/night) and 70% rh in a greenhouse. Efficacy was assessed by visual evaluation directly when an appropriate level of disease appears on untreated check plants (usually >80% disease cover at 16-19 days after inoculation).

For infection with Puccinia recondita: The test plants were inoculated by spraying them with a spore suspension 1 day after application (spore suspension at 80′000 spores per ml in water supplemented with Tween® 20 at 0.1%). After an incubation period of 1 day at 20 °C and 95% rh, the inoculated test plants were kept at 20 °C and 60% rh in a greenhouse. The percentage leaf area covered by disease was assessed by visual evaluation when an appropriate level of disease appeared on untreated check plants (usually 50-80% disease cover at 9-12 days after infection).

The three segments of the leaf were evaluated individually.

TABLE 6
Disease control efficacy of a compound according to Formula
I(a) and I(b) in leaf painting assay in glasshouse

| | | | Puccinia | |
|---|---|---|---|---|
| | concen- | | recondita | Zymoseptoria |
| | tration | | f. sp. tritici | tritici |
| compound | (ppm) | segment | % reduction of fungal growth | |
| Formula I (a) | 200 | middle | 61 | 46 |
| | 20 | middle | 70 | 0 |
| Formula I (b) | 200 | middle | 67 | 31 |
| | 20 | middle | 55 | 0 |
| benzovindiflupyr | 188.00 | middle | 100 | 100 |
| Water | — | middle | 0 | 0 |

The results in Table 6 show that compounds of Formula I(a) and Formula I(b) were active in the assay providing up to 70% control against Puccinia recondite and Zymoseptoria tritici on the treated area (middle segment). Compounds of Formula I(a) and Formula I(b) have similar efficacy.

3.4. Reactive Oxygen Species (ROS) Burst Assay with Wheat Leaf Discs

200 μL of the test solution (analyte) or the respective control (water) in the appropriate concentration was pipetted into a white 96-well plate (Nunc, Langenselbold, Germany). 5 mm leaf discs from 2-week-old wheat plants were obtained using a tissue punch and subsequently floated on the test solution. The plates were stored for 24 h at Room Temperature (RT). The next day, the solution was replaced with 50 μL ddH₂O and leaf discs were left for regeneration for at least one hour at RT in the dark. Meanwhile appropriate master mixes, either with or without the elicitor flg22 (see below), were freshly prepared in black 5 mL reaction tubes. After regeneration, 50 μL of the corresponding master mix was added to the leaf disc containing wells. Subsequently, luminescence was recorded for 40 minutes with a platereader (BMG Labtech; Ortenberg, Germany). The compound INA (2.6-dichloro-isonicotinic acid, CAS: 5398-44-7) is a synthetic salicylic acid analog and was included as a reference for priming activity (Kauss et al., 1992).

Mastermix−flg22: 4.98 mL ddH2O, 10 μL HRP (10 mg/mL), 10 μL L-012 (20 mM)

Mastermix+flg22: 4.979 mL ddH2O, 10 μL HRP (10 mg/mL), 10 μL L-012 (20 mM), 1 μL flg22 (10 μM) Abbreviations: flg22 (22 amino acids flagellin peptide. Eurogentec Cat. Number AS-62633); HRP (Horseradish Peroxidase), used L-012 sodium salt (CAS #: 143556-24-5).

TABLE 7
Fold change of peak ROS production. Numbers report the ratio
of peak values observed for treated vs. control, using the
maximum value each condition measured over a time-course of
40 minutes. Values indicated are the mean of two repetitions.

| | Concentration | Fold change |
|---|---|---|
| Sample | (ppm) | (peak ROS) |
| INA | 100 | 1.8 |
| compound formula I(a) | 100 | 2.7 |
| | 25 | 0.8 |
| | 1 | 0.8 |
| compound formula I(b) | 100 | 3.0 |
| | 25 | 1.3 |
| | 1 | 1.2 |

The results in Table 7 demonstrate that wheat pretreated with a compound of Formula I(a) or Formula I(b) in a concentration of 100 ppm increased ROS production induced by the peptide flg22 by a factor of 2.7 or higher. This is a response that is similar to or stronger than the response observed from a treatment with INA (2.6-dichloro-isonicotinic acid), a well-known priming agent (Krauss et al., 1992: Dichloroisonicotinic and salicylic acid, inducers of systemic acquired resistance, enhance fungal elicitor responses in parsley cells, Plant Journal 2:655-60).

### Example 4. Fungicidal Activity of a Mixture of a Lipopeptide According to Formula I(a) and Malonomicin in Liquid Culture Assays

### Method

A stock solution for compound of Formula I(a) was produced in DMSO (max. 10 mg/ml). Malonomicin was produced according to Law et al, 2018 (Nature Catalys is |VOL 1|DECEMBER 2018|977-984). A stock solution of malonomicin was produced in water plus 0.025% Tween® 20.

Assays to test efficacy of mixtures of the lipopeptide compound of Formula I(a) in combination with malonomicin on the control of fungal pathogens in a liquid culture assay were designed for a 96well plate as outlined in table 1:

TABLE 8
Outline of 96 well testplate including concentration of compounds per well. The upper number in
the cell indicates malonomicin concentration (ppm), the lower number compound of Formula I(a)
concentration (ppm). Column (1) holds a dilution series of compound of Formula I(a), row (H)
holds a dilution series of malonomicin, column (12) hold cells representing untreated check

| ppm | (1) | (2) | (3) | (4) | (5) | (6) | (7) | (8) | (9) | (10) | (11) | (12) |
|---|---|---|---|---|---|---|---|---|---|---|---|---|
| A | 0 | 1000 | 500 | 250 | 125 | 62.5 | 31.25 | 15.63 | 7.81 | 3.00 | 1.00 | 0 |
| | 100 | 100 | 100 | 100 | 100 | 100 | 100 | 100 | 100 | 100 | 100 | 0 |
| B | 0 | 1000 | 500 | 250 | 125 | 62.5 | 31.25 | 15.63 | 7.81 | 3.00 | 1.00 | 0 |
| | 50 | 50 | 50 | 50 | 50 | 50 | 50 | 50 | 50 | 50 | 50 | 0 |
| C | 0 | 1000 | 500 | 250 | 125 | 62.5 | 31.25 | 15.63 | 7.81 | 3.00 | 1.00 | 0 |
| | 25 | 25 | 25 | 25 | 25 | 25 | 25 | 25 | 25 | 25 | 25 | 0 |
| D | 0 | 1000 | 500 | 250 | 125 | 62.5 | 31.25 | 15.63 | 7.81 | 3.00 | 1.00 | 0 |
| | 12.5 | 12.5 | 12.5 | 12.5 | 12.5 | 12.5 | 12.5 | 12.5 | 12.5 | 12.5 | 12.5 | 0 |
| E | 0 | 1000 | 500 | 250 | 125 | 62.5 | 31.25 | 15.63 | 7.81 | 3.00 | 1.00 | 0 |
| | 6.25 | 6.25 | 6.25 | 6.25 | 6.25 | 6.25 | 6.25 | 6.25 | 6.25 | 6.25 | 6.25 | 0 |
| F | 0 | 1000 | 500 | 250 | 125 | 62.5 | 31.25 | 15.63 | 7.81 | 3.00 | 1.00 | 0 |
| | 3.125 | 3.125 | 3.125 | 3.125 | 3.125 | 3.125 | 3.125 | 3.125 | 3.125 | 3.125 | 3.125 | 0 |
| G | 0 | 1000 | 500 | 250 | 125 | 62.5 | 31.25 | 15.63 | 7.81 | 3.00 | 1.00 | 0 |
| | 1.56 | 1.56 | 1.56 | 1.56 | 1.56 | 1.56 | 1.56 | 1.56 | 1.56 | 1.56 | 1.56 | 0 |
| H | 0 | 1000 | 500 | 250 | 125 | 62.5 | 31.25 | 15.63 | 7.81 | 3.00 | 1.00 | 0 |
| | 0 | 0 | 0 | 0 | 0 | 0 | 0 | 0 | 0 | 0 | 0 | 0 |

The 96 well plate design allows to compare disease control of the mixture with disease control of the respective single compound at the same rate. The comparison of the assessed efficacy from a mixture with the calculated efficacy of the same mixture according to Colby allows a statement if a mixture is additive (efficacy is similar to Colby calculation), synergistic (efficacy is better than Colby calculation) or antagonistic (efficacy is inferior to Colby calculation).

The same 96 well plate design also allows to assess if the two compounds can be mixed at different use rates and mixture ratios, the plate design outlined in table 1 will provide the following mixture ratios, as shown in table 2.

TABLE 9
Mixture ratio of two compounds in the 96well plate assay design as outlined in Table 8. The number
represents the ratio of compound 1:compound 2. Compound 1 is malonomicin, and compound 2 is the
lipopeptyide compound of Formula 1(a). The plate design spans a wide range from 641:1 to 1:100.

| | | | | | | | | | | | |
|---|---|---|---|---|---|---|---|---|---|---|---|
| (1) | (2) | (3) | (4) | (5) | (6) | (7) | (8) | (9) | (10) | (11) | (12) |

| | | | | | | | | | | |
|---|---|---|---|---|---|---|---|---|---|---|
| A | 10:1 | 5:1 | 2.5:1 | 1.25:1 | 1:1.6 | 1:3.2 | 1:6 | 1:13 | 1:33 | 1:100 |
| B | 20:1 | 10:1 | 5:1 | 2.5:1 | 1.25:1 | 1:1.6 | 1:3.2 | 1:6 | 1:17 | 1:50 |
| C | 40:1 | 20:1 | 10:1 | 5:1 | 2.5:1 | 1.25:1 | 1:1.6 | 1:3.2 | 1:8 | 1:25 |
| D | 80:1 | 40:1 | 20:1 | 10:1 | 5:1 | 2.5:1 | 1.25:1 | 1:1.6 | 1:4.2 | 1:13 |
| E | 160:1 | 80:1 | 40:1 | 20:1 | 10:1 | 5:1 | 2.5:1 | 1.25:1 | 1:2.1 | 1:6 |
| F | 320:1 | 160:1 | 80:1 | 40:1 | 20:1 | 10:1 | 5:1 | 2.5:1 | 1:1 | 1:3.1 |
| G | 641:1 | 321:1 | 160:1 | 80:1 | 40:1 | 20:1 | 10:1 | 5:1 | 1.9:1 | 1:1.6 |
| H | | | | | | | | | | |

A master plate with 10× concentrated solution of the compound stock solutions diluted in water plus 0.025% Tween® 20 was prepared. The concentration of DMSO (from the stock of compound of Formula (a)) and Tween® 20 was kept constant in all cells of the master plate. 10 μl was transferred from the master plate to a 96well test plate. The nutrient broth containing the fungal spores/mycelia fragments was then added to the test plate to give an end concentration of 1× for the tested compounds (as outlined in Table 8). The test plates were incubated in the dark at 24 °C and 96% rh. The inhibition of fungal growth was determined photometrically after ca 3 days, and percent fungal growth reduction relative to the untreated check was calculated.

Efficacy of the mixtures was tested on different fugnal species:

Zymoseptoria tritici (EPPO Code: SEPTTR)

Conidia of the fungus from cryogenic storage were directly mixed into nutrient broth (PDB: potato dextrose broth). The test plates were incubated at 24 °C and the inhibition of growth was determined photometrically after 72 hrs.

Fusarium culmorum (EPPO Code: FUSACU)

Conidia of the fungus from cryogenic storage were directly mixed into nutrient broth (PDB: potato dextrose broth). The test plates were incubated at 24 °C and the inhibition of growth was determined photometrically after 72 hrs.

Microdochium nivale (EPPO Code: MONGNI)

Conidia of the fungus from cryogenic storage were directly mixed into nutrient broth (PDB: potato dextrose broth). The test plates were incubated at 24 °C and the inhibition of growth was determined photometrically after 72 hrs.

Botrytis cinerea (EPPO Code: BOTRCI)

Conidia of the fungus from cryogenic storage were directly mixed into nutrient broth (Vogel's minimal media). The test plates were incubated at 24 °C and the inhibition of growth was determined photometrically after 72 hrs.

Pyricularia oryzae (EPPO Code: PYRIOR)

Conidia of the fungus from cryogenic storage were directly mixed into nutrient broth (PDB: potato dextrose broth). The test plates were incubated at 24 °C and the inhibition of growth was determined photometrically after 72 hrs.

Cercospora arachidicola (EPPO Code: MYCOAR)

Conidia of the fungus from cryogenic storage were directly mixed into nutrient broth (PDB: potato dextrose broth). The test plates were incubated at 24 °C and the inhibition of growth was determined photometrically after 5-6 days.

Sclerotinia sclerotiorum (EPPO Code: SCLESC)

Mycelial fragments of the fungus prepared from a fresh liquid culture were directly mixed into nutrient broth (PDB: potato dextrose broth). The test plates were incubated at 24 °C and the inhibition of growth was determined photometrically after 72 hrs.

### Results

TABLE 10
Control of Zymoseptoria tritici for solo compounds and mixtures. The plate
design including the concentration of compound of Formula I(a) and malonomicin
are indicated in Table 8. Values indicate control of fungal growth (% reduction
of growth in the test well as compared to untreated check).

| | (1) | (2) | (3) | (4) | (5) | (6) | (7) | (8) | (9) | (10) | (11) | (12) |
|---|---|---|---|---|---|---|---|---|---|---|---|---|
| A | 90 | 100 | 100 | 100 | 100 | 100 | 100 | 100 | 90 | 90 | 90 | 0 |
| B | 90 | 100 | 100 | 100 | 100 | 100 | 100 | 100 | 90 | 90 | 90 | 0 |
| C | 70 | 100 | 100 | 100 | 100 | 100 | 100 | 90 | 70 | 70 | 70 | 0 |
| D | 70 | 100 | 100 | 100 | 100 | 100 | 100 | 90 | 70 | 70 | 70 | 0 |
| E | 50 | 100 | 100 | 100 | 90 | 90 | 70 | 50 | 50 | 50 | 50 | 0 |
| F | 20 | 100 | 100 | 70 | 70 | 70 | 50 | 20 | 20 | 20 | 20 | 0 |
| G | 0 | 100 | 100 | 70 | 70 | 50 | 20 | 20 | 20 | 0 | 0 | 0 |
| H | 0 | 100 | 90 | 70 | 70 | 50 | 20 | 20 | 0 | 0 | 0 | 0 |

For each of the assay conditions in Table 10 with efficacy of 50% or more (from here onwards termed: effective mixtures) the respective mixture ratio can be assigned from Table 9. Effective mixtures for the control of Zymoseptoria tritici were found from 641:1 to 1:100.

TABLE 11
Comparison of measured values for disease control of Zymoseptoria tritici
(as reported in Table 10) with calculated values using the formula
from Colby for the same mixture. Numbers reported in the table represent
the difference from measured efficacy (in %) minus calculated efficacy
(in %). Values around 0 (zero) indicate additive activity, while
positive values suggest synergistic activity.

| | | | | | | | | | | | |
|---|---|---|---|---|---|---|---|---|---|---|---|
| (1) | (2) | (3) | (4) | (5) | (6) | (7) | (8) | (9) | (10) | (11) | (12) |

| | | | | | | | | | | |
|---|---|---|---|---|---|---|---|---|---|---|
| A | 0 | 1 | 3 | 3 | 5 | 8 | 8 | 0 | 0 | 0 |
| B | 0 | 1 | 3 | 3 | 5 | 8 | 8 | 0 | 0 | 0 |
| C | 0 | 3 | 9 | 9 | 15 | 24 | 14 | 0 | 0 | 0 |
| D | 0 | 3 | 9 | 9 | 15 | 24 | 14 | 0 | 0 | 0 |
| E | 0 | 5 | 15 | 5 | 15 | 10 | −10 | 0 | 0 | 0 |
| F | 0 | 8 | −6 | −6 | 10 | 14 | −16 | 0 | 0 | 0 |
| G | 0 | 10 | 0 | 0 | 0 | 0 | 0 | 20 | 0 | 0 |
| H | | | | | | | | | | |

TABLE 12
Control of Fusarium culmorum for solo compounds and mixtures.
The plate design including the concentration of compound
of Formula I(a) and malonomicin are indicated in Table 8.
Values indicate control of fungal growth (% reduction of
growth in the test well as compared to untreated check).

| | (1) | (2) | (3) | (4) | (5) | (6) | (7) | (8) | (9) | (10) | (11) | (12) |
|---|---|---|---|---|---|---|---|---|---|---|---|---|
| A | 0 | 20 | 20 | 20 | 20 | 0 | 0 | 0 | 0 | 0 | 0 | 0 |
| B | 0 | 50 | 20 | 20 | 20 | 0 | 0 | 0 | 0 | 0 | 0 | 0 |
| C | 0 | 70 | 20 | 20 | 20 | 20 | 20 | 20 | 20 | 0 | 0 | 0 |
| D | 0 | 90 | 50 | 20 | 20 | 20 | 20 | 20 | 20 | 0 | 0 | 0 |
| E | 0 | 90 | 70 | 20 | 20 | 20 | 20 | 20 | 20 | 0 | 0 | 0 |
| F | 0 | 70 | 50 | 20 | 20 | 20 | 20 | 20 | 0 | 0 | 0 | 0 |
| G | 0 | 50 | 20 | 20 | 20 | 0 | 0 | 0 | 0 | 0 | 0 | 0 |
| H | 0 | 20 | 20 | 20 | 20 | 0 | 0 | 0 | 0 | 0 | 0 | 0 |

For each of the assay conditions in Table 12 with efficacy of 50% or more (effective mixture) the respective mixture ratio can be assigned from Table 9. Effective mixtures for the control of Fusarium culmorum were found from 641:1 to 20:1.

TABLE 13
Comparison of measured values for disease control of
Fusarium culmorum (as reported in Table 12) with calculated
values using the formula from Colby for the same mixture.
Numbers reported in the table represent the difference
from measured efficacy (in %) minus calculated efficacy
(in %). Values around 0 (zero) indicate additive activity,
while positive values suggest synergistic activity.

| | | | | | | | | | | | |
|---|---|---|---|---|---|---|---|---|---|---|---|
| (1) | (2) | (3) | (4) | (5) | (6) | (7) | (8) | (9) | (10) | (11) | (12) |

| | | | | | | | | | | |
|---|---|---|---|---|---|---|---|---|---|---|
| A | 0 | 0 | 0 | 0 | 0 | 0 | 0 | 0 | 0 | 0 |
| B | 30 | 0 | 0 | 0 | 0 | 0 | 0 | 0 | 0 | 0 |
| C | 50 | 0 | 0 | 0 | 20 | 20 | 20 | 20 | 0 | 0 |
| D | 70 | 30 | 0 | 0 | 20 | 20 | 20 | 20 | 0 | 0 |
| E | 70 | 50 | 0 | 0 | 20 | 20 | 20 | 20 | 0 | 0 |
| F | 50 | 30 | 0 | 0 | 20 | 20 | 20 | 0 | 0 | 0 |
| G | 30 | 0 | 0 | 0 | 0 | 0 | 0 | 0 | 0 | 0 |
| H | | | | | | | | | | |

TABLE 14
Control of Microdochium nivale for solo compounds and mixtures. The plate
design including the concentration of compound of Formula I(a) and malonomicin
are indicated in Table 8. Values indicate control of fungal growth (% reduction
of growth in the test well as compared to untreated check)

| | (1) | (2) | (3) | (4) | (5) | (6) | (7) | (8) | (9) | (10) | (11) | (12) |
|---|---|---|---|---|---|---|---|---|---|---|---|---|
| A | 0 | 100 | 100 | 100 | 100 | 70 | 50 | 20 | 20 | 20 | 20 | 0 |
| B | 0 | 100 | 100 | 100 | 100 | 70 | 50 | 20 | 20 | 20 | 20 | 0 |
| C | 0 | 100 | 100 | 100 | 100 | 70 | 50 | 20 | 20 | 20 | 20 | 0 |
| D | 0 | 100 | 100 | 100 | 90 | 70 | 50 | 20 | 20 | 20 | 20 | 0 |
| E | 0 | 100 | 100 | 100 | 90 | 70 | 50 | 20 | 20 | 20 | 20 | 0 |
| F | 0 | 100 | 100 | 100 | 90 | 70 | 50 | 20 | 20 | 20 | 20 | 0 |
| G | 0 | 100 | 100 | 100 | 90 | 70 | 50 | 20 | 20 | 20 | 20 | 0 |
| H | 0 | 100 | 100 | 100 | 90 | 50 | 20 | 20 | 20 | 20 | 20 | 0 |

For each of the assay conditions in Table 14 with efficacy of 50% or more (effective mixture) the respective mixture ratio can be assigned from Table 9. Effective mixtures for the control of Microdochium nivale were found from 641:1 to 1:3.

TABLE 15
Comparison of measured values for disease control of Microdochium
nivale (as reported in Table 14) with calculated values using the
formula from Colby for the same mixture. Numbers reported in the table
represent the difference from measured efficacy (in %) minus calculated
efficacy (in %). Values around 0 (zero) indicate additive activity,
while positive values suggest synergistic activity.

| | | | | | | | | | | | |
|---|---|---|---|---|---|---|---|---|---|---|---|
| (1) | (2) | (3) | (4) | (5) | (6) | (7) | (8) | (9) | (10) | (11) | (12) |

| | | | | | | | | | | |
|---|---|---|---|---|---|---|---|---|---|---|
| A | 0 | 0 | 0 | 10 | 20 | 30 | 0 | 0 | 0 | 0 |
| B | 0 | 0 | 0 | 10 | 20 | 30 | 0 | 0 | 0 | 0 |
| C | 0 | 0 | 0 | 10 | 20 | 30 | 0 | 0 | 0 | 0 |
| D | 0 | 0 | 0 | 0 | 20 | 30 | 0 | 0 | 0 | 0 |
| E | 0 | 0 | 0 | 0 | 20 | 30 | 0 | 0 | 0 | 0 |
| F | 0 | 0 | 0 | 0 | 20 | 30 | 0 | 0 | 0 | 0 |
| G | 0 | 0 | 0 | 0 | 20 | 30 | 0 | 0 | 0 | 0 |
| H | | | | | | | | | | |

TABLE 16
Control of Botrytis cinerea for solo compounds and mixtures.
The plate design including the concentration of the lipopeptide
compound of Formula I(a) and malonomicin are indicated in Table
8. Values indicate control of fungal growth (% reduction of growth
in the test well as compared to untreated check).

| | (1) | (2) | (3) | (4) | (5) | (6) | (7) | (8) | (9) | (10) | (11) | (12) |
|---|---|---|---|---|---|---|---|---|---|---|---|---|
| A | 20 | 100 | 90 | 90 | 70 | 70 | 50 | 50 | 20 | 20 | 20 | 0 |
| B | 20 | 100 | 90 | 90 | 70 | 70 | 50 | 50 | 20 | 0 | 0 | 0 |
| C | 0 | 100 | 90 | 70 | 70 | 70 | 50 | 20 | 20 | 0 | 0 | 0 |
| D | 0 | 90 | 90 | 70 | 70 | 70 | 50 | 20 | 20 | 0 | 0 | 0 |
| E | 0 | 90 | 90 | 70 | 70 | 70 | 50 | 20 | 0 | 0 | 0 | 0 |
| F | 0 | 90 | 70 | 70 | 70 | 70 | 50 | 20 | 0 | 0 | 0 | 0 |
| G | 0 | 90 | 70 | 70 | 70 | 70 | 50 | 20 | 0 | 0 | 0 | 0 |
| H | 0 | 70 | 70 | 70 | 70 | 70 | 20 | 0 | 0 | 0 | 0 | 0 |

For each of the assay conditions in Table 16 with efficacy of 50% or more (effective mixture) the respective mixture ratio can be assigned from Table 9. Effective mixtures for the control of Botrytis cinerea were found from 641:1 to 1:6.

TABLE 17
Comparison of measured values for disease control of Botrytis cinerea
(as reported in Table 9) with calculated values using the formula
from Colby for the same mixture. Numbers reported in the table represent
the difference from measured efficacy (in %) minus calculated efficacy
(in %). Values around 0 (zero) indicate additive activity, while
positive values suggest synergistic activity.

| | | | | | | | | | | | |
|---|---|---|---|---|---|---|---|---|---|---|---|
| (1) | (2) | (3) | (4) | (5) | (6) | (7) | (8) | (9) | (10) | (11) | (12) |

| | | | | | | | | | | |
|---|---|---|---|---|---|---|---|---|---|---|
| A | 24 | 14 | 14 | −6 | −6 | 14 | 30 | 0 | 0 | 0 |
| B | 24 | 14 | 14 | −6 | −6 | 14 | 30 | 0 | −20 | −20 |
| C | 30 | 20 | 0 | 0 | 0 | 30 | 20 | 20 | 0 | 0 |
| D | 20 | 20 | 0 | 0 | 0 | 30 | 20 | 20 | 0 | 0 |
| E | 20 | 20 | 0 | 0 | 0 | 30 | 20 | 0 | 0 | 0 |
| F | 20 | 0 | 0 | 0 | 0 | 30 | 20 | 0 | 0 | 0 |
| G | 20 | 0 | 0 | 0 | 0 | 30 | 20 | 0 | 0 | 0 |
| H | | | | | | | | | | |

TABLE 18
Control of Pyricularia oryzae for solo compounds and mixtures.
The plate design including the concentration of the lipopeptide
compound of Formula I(a) and malonomicin are indicated in Table
8. Values indicate control of fungal growth (% reduction of
growth in the test well as compared to untreated check)

| | (1) | (2) | (3) | (4) | (5) | (6) | (7) | (8) | (9) | (10) | (11) | (12) |
|---|---|---|---|---|---|---|---|---|---|---|---|---|
| A | 20 | 100 | 100 | 90 | 90 | 70 | 50 | 20 | 20 | 20 | 20 | 0 |
| B | 20 | 100 | 100 | 90 | 90 | 50 | 50 | 20 | 20 | 20 | 20 | 0 |
| C | 20 | 100 | 100 | 90 | 70 | 50 | 50 | 20 | 20 | 20 | 20 | 0 |
| D | 20 | 100 | 90 | 90 | 70 | 50 | 50 | 20 | 20 | 20 | 20 | 0 |
| E | 20 | 100 | 90 | 90 | 70 | 50 | 50 | 20 | 20 | 20 | 20 | 0 |
| F | 20 | 100 | 90 | 90 | 70 | 50 | 50 | 20 | 20 | 20 | 20 | 0 |
| G | 20 | 100 | 90 | 90 | 70 | 50 | 50 | 20 | 20 | 20 | 20 | 0 |
| H | 0 | 90 | 70 | 70 | 50 | 50 | 20 | 20 | 0 | 0 | 0 | 0 |

For each of the assay conditions in Table 18 with efficacy of 50% or more (effective mixture) the respective mixture ratio can be assigned from Table 9. Effective mixtures for the control of Pyricularia oryzae were found from 641:1 to 1:3.

TABLE 19
Comparison of measured values for disease control of Pyricularia oryzae
(as reported in Table 18) with calculated values using the formula
from Colby for the same mixture. Numbers reported in the table represent
the difference from measured efficacy (in %) minus calculated efficacy
(in %). Values around 0 (zero) indicate additive activity, while
positive values suggest synergistic activity.

| | | | | | | | | | | | |
|---|---|---|---|---|---|---|---|---|---|---|---|
| (1) | (2) | (3) | (4) | (5) | (6) | (7) | (8) | (9) | (10) | (11) | (12) |

| | | | | | | | | | | |
|---|---|---|---|---|---|---|---|---|---|---|
| A | 8 | 24 | 14 | 30 | 10 | 14 | −16 | 0 | 0 | 0 |
| B | 8 | 24 | 14 | 30 | −10 | 14 | −16 | 0 | 0 | 0 |
| C | 8 | 24 | 14 | 10 | −10 | 14 | −16 | 0 | 0 | 0 |
| D | 8 | 14 | 14 | 10 | −10 | 14 | −16 | 0 | 0 | 0 |
| E | 8 | 14 | 14 | 10 | −10 | 14 | −16 | 0 | 0 | 0 |
| F | 8 | 14 | 14 | 10 | −10 | 14 | −16 | 0 | 0 | 0 |
| G | 8 | 14 | 14 | 10 | −10 | 14 | −16 | 0 | 0 | 0 |
| H | | | | | | | | | | |

TABLE 20
Control of Cercospora arachidicola for solo compounds and mixtures. The
plate design including the concentration of compound of Formula I(a) and
malonomicin are indicated in Table 8. Values indicate control of fungal growth
(% reduction of growth in the test well as compared to untreated check)

| | (1) | (2) | (3) | (4) | (5) | (6) | (7) | (8) | (9) | (10) | (11) | (12) |
|---|---|---|---|---|---|---|---|---|---|---|---|---|
| A | 90 | 100 | 100 | 100 | 100 | 100 | 100 | 100 | 100 | 100 | 100 | 0 |
| B | 90 | 100 | 100 | 100 | 100 | 100 | 90 | 90 | 90 | 90 | 90 | 0 |
| C | 90 | 100 | 100 | 100 | 100 | 100 | 90 | 90 | 90 | 90 | 90 | 0 |
| D | 90 | 100 | 100 | 100 | 100 | 90 | 90 | 90 | 90 | 90 | 90 | 0 |
| E | 70 | 100 | 100 | 90 | 90 | 90 | 70 | 70 | 70 | 70 | 70 | 0 |
| F | 20 | 100 | 90 | 90 | 70 | 70 | 50 | 20 | 20 | 20 | 20 | 0 |
| G | 0 | 100 | 90 | 70 | 70 | 70 | 50 | 20 | 20 | 0 | 0 | 0 |
| H | 0 | 100 | 90 | 70 | 70 | 50 | 50 | 20 | 20 | 0 | 0 | 0 |

For each of the assay conditions in Table 20 with efficacy of 50% or more (effective mixture) the respective mixture ratio can be assigned from Table 9. Effective mixtures for the control of Cercospora arachidicola were found from 641:1 to 1:100.

TABLE 21
Control of Sclerotinia sclerotiorum for solo compounds and
mixtures. The plate design including the concentration of
compound of Formula I(a) and malonomicin are indicated in
Table 8. Values indicate control of fungal growth (% reduction
of growth in the test well as compared to untreated check)

| | (1) | (2) | (3) | (4) | (5) | (6) | (7) | (8) | (9) | (10) | (11) | (12) |
|---|---|---|---|---|---|---|---|---|---|---|---|---|
| A | 0 | 90 | 90 | 90 | 70 | 50 | 20 | 20 | 0 | 0 | 0 | 0 |
| B | 0 | 90 | 90 | 90 | 70 | 50 | 20 | 20 | 0 | 0 | 0 | 0 |
| C | 0 | 90 | 90 | 90 | 70 | 50 | 20 | 20 | 0 | 0 | 0 | 0 |
| D | 0 | 90 | 90 | 90 | 70 | 50 | 20 | 0 | 0 | 0 | 0 | 0 |
| E | 0 | 90 | 90 | 90 | 70 | 50 | 20 | 0 | 0 | 0 | 0 | 0 |
| F | 0 | 90 | 90 | 90 | 70 | 50 | 20 | 0 | 0 | 0 | 0 | 0 |
| G | 0 | 90 | 90 | 90 | 70 | 50 | 20 | 0 | 0 | 0 | 0 | 0 |
| H | 0 | 90 | 90 | 90 | 70 | 50 | 20 | 0 | 0 | 0 | 0 | 0 |

For each of the assay conditions in Table 21 with efficacy of 50% or more (effective mixture) the respective mixture ratio can be assigned from Table 9. Effective mixtures for the control of Sclerotinia sclerotiorum were found from 641:1 to 1:2.

### Conclusion

A mixture of the lipopeptide compound of Formula I(a) and malonomicin controls various fungal pathogens. A large variety of mixture ratios of the two compounds produces 50% or more control of the fungal growth, ranging from 641:1 to 1:100, for several fungal species, including examples with a ratio in between the mentioned ratios (ratio of compounds malonomicin:compound of Formula I(a)).

Surprisingly, the efficacy of many mixtures is better than the efficacy predicted based on the calculation from Colby, indicating that the mixture of the lipopeptide compound of Formula I(a) with malonomicin has a synergistic effect on the control of fungal pathogens. This surprising synergistic effect was observed in tests to control Zymoseptoria tritici, Fusarium culmorum, Microdochium nivale, Botrytis cinerea and Pyricularia oryzae.

### Example 5. Fungicidal Activity of a Mixture of the Lipopeptide Compounds According to Formula I(a) and Malonomicin in Leaf Disc Assays

A stock solution for the lipopeptide compound of Formula I(a) was produced in DMSO (max. 10 mg/ml). Malonomicin was produced according to Law et al, 2018 (Nature Catalys is |VOL 1|DECEMBER 2018|977-984). A stock solution of malonomicin was produced in water plus 0.025% Tween®20.

Assays to test efficacy of mixtures of compound of Formula I(a) in combination with malonomicin on the control of fungal pathogens in a leaf disc assay were designed for two 24well plates.

Table 22 and Table 23. Outline of 24well testplate (1) and testplate (2) including concentration of compounds sprayed per well. The upper number in the cell indicates compound of Formula I(a) concentration (ppm), the lower number malonomicin concentration (ppm). Column (1) holds a dilution series of compound of Formula I(a), row (2-D) hold a dilution series of malonomicin. Well 2-D-(1) represents an untreated check. This design was applied for tests including Puccinia recondita (EPPO code: PUCCRE) with preventive and curative spray timing.

24 well plate (1)

| ppm | (1) | (2) | (3) | (4) | (5) | (6) |
|---|---|---|---|---|---|---|
| 1-A | 200 | 200 | 200 | 200 | 200 | 200 |
| | 0 | 200 | 20 | 6.67 | 2.22 | 0.74 |
| 1-B | 100 | 100 | 100 | 100 | 100 | 100 |
| | 0 | 200 | 20 | 6.67 | 2.22 | 0.74 |
| 1-C | 50 | 50 | 50 | 50 | 50 | 50 |
| | 0 | 200 | 20 | 6.67 | 2.22 | 0.74 |
| 1-D | 25 | 25 | 25 | 25 | 25 | 25 |
| | 0 | 200 | 20 | 6.67 | 2.22 | 0.74 |

24 well plate (2)

| ppm | (1) | (2) | (3) | (4) | (5) | (6) |
|---|---|---|---|---|---|---|
| 2-A | 12.5 | 12.5 | 12.5 | 12.5 | 12.5 | 12.5 |
| | 0 | 200 | 20 | 6.67 | 2.22 | 0.74 |
| 2-B | 6.25 | 6.25 | 6.25 | 6.25 | 6.25 | 6.25 |
| | 0 | 200 | 20 | 6.67 | 2.22 | 0.74 |
| 2-C | 3.13 | 3.13 | 3.13 | 3.13 | 3.13 | 3.13 |
| | 0 | 200 | 20 | 6.67 | 2.22 | 0.74 |
| 2-D | 0 | 0 | 0 | 0 | 0 | 0 |
| | 0 | 200 | 20 | 6.67 | 2.22 | 0.74 |

TABLE 24
Mixture ratio of compounds sprayed in the 24well plate
assay as outlined in Table 22 and Table 23. The number
represents the ratio of compound 1:compound 2. Compound
1 is malonomicin, and compound 2 is compound of Formula
I(a). The plate design across the two 24well plates
spans a wide range from 64:1 up to 1:270.

| | | | | | |
|---|---|---|---|---|---|
| (1) | (2) | (3) | (4) | (5) | (6) |

| | | | | | |
|---|---|---|---|---|---|
| 1-A | 1:1 | 1:10 | 1:30 | 1:90 | 1:270 |
| 1-B | 2:1 | 1:5 | 1:15 | 1:45 | 1:135 |
| 1-C | 4:1 | 1:2.5 | 1:7.5 | 1:23 | 1:68 |
| 1-D | 8:1 | 1:1.3 | 1:3.7 | 1:11 | 1:34 |
| 2-A | 16:1 | 1.6:1 | 1:1.9 | 1:5.6 | 1:17 |
| 2-B | 32:1 | 3.2:1 | 1.1:1 | 1:2.8 | 1:8.4 |
| 2-C | 64:1 | 6.4:1 | 2.1:1 | 1:1.4 | 1:4.2 |
| 2-D | | | | | |

Table 25 and 26. Outline of 24well testplate (3) and testplate (4) including concentration of compounds per well. The upper number in the cell indicates the lipopeptide compound of Formula I(a) concentration (ppm), the lower number malonomicin concentration (ppm). Column (1) holds a dilution series of compound of Formula I(a), row (4-D) hold a dilution series of malonomicin. Well 4-D-(1) represents an untreated check. This design was applied for tests including Blumeria graminis f.sp. tritici (EPPO code: ERYSGT) with preventive spray timing and Parastagonospora nodorum (EPPO code: LEPTNO) with preventive spray timing.

24 well plate (3)

| ppm | (1) | (2) | (3) | (4) | (5) | (6) |
|---|---|---|---|---|---|---|
| 3-A | 200 | 200 | 200 | 200 | 200 | 200 |
| | 0 | 600 | 200 | 66.7 | 22.2 | 7.4 |
| 3-B | 100 | 100 | 100 | 100 | 100 | 100 |
| | 0 | 600 | 200 | 66.7 | 22.2 | 7.4 |
| 3-C | 50 | 50 | 50 | 50 | 50 | 50 |
| | 0 | 600 | 200 | 66.7 | 22.2 | 7.4 |
| 3-D | 25 | 25 | 25 | 25 | 25 | 25 |
| | 0 | 600 | 200 | 66.7 | 22.2 | 7.4 |

24 well plate (4)

| ppm | (1) | (2) | (3) | (4) | (5) | (6) |
|---|---|---|---|---|---|---|
| 4-A | 12.5 | 12.5 | 12.5 | 12.5 | 12.5 | 12.5 |
| | 0 | 600 | 200 | 66.7 | 22.2 | 7.4 |
| 4-B | 6.25 | 6.25 | 6.25 | 6.25 | 6.25 | 6.25 |
| | 0 | 600 | 200 | 66.7 | 22.2 | 7.4 |
| 4-C | 3.13 | 3.13 | 3.13 | 3.13 | 3.13 | 3.13 |
| | 0 | 600 | 200 | 66.7 | 22.2 | 7.4 |
| 4-D | 0 | 0 | 0 | 0 | 0 | 0 |
| | 0 | 600 | 200 | 66.7 | 22.2 | 7.4 |

TABLE 27
Mixture ratio of compounds sprayed in the 24well plate
assay as outlined in Table 25 and Table 26. The number
represents the ratio of compound 1:compound 2. Compound
1 is malonomicin, and compound 2 is the lipopeptide compound
of Formula I(a). The plate design across the two 24well
plates spans a wide range from 192:1 up to 1:27.

| | | | | | |
|---|---|---|---|---|---|
| (1) | (2) | (3) | (4) | (5) | (6) |

| | | | | | |
|---|---|---|---|---|---|
| 3-A | 3:1 | 1:1 | 1:3 | 1:9 | 1:27 |
| 3-B | 6:1 | 2:1 | 1:1.5 | 1:4.5 | 1:13 |
| 3-C | 12:1 | 4:1 | 1.3:1 | 1:2.3 | 1:6.8 |
| 3-D | 24:1 | 8:1 | 2.7:1 | 1:1.1 | 1:3.4 |
| 4-A | 48:1 | 16:1 | 5.3:1 | 1.8:1 | 1:1.7 |
| 4-B | 96:1 | 32:1 | 11:1 | 3.6:1 | 1.2:1 |
| 4-C | 192:1 | 64:1 | 21:1 | 7.1:1 | 2.4:1 |
| 4-D | | | | | |

A first set of master plates with 1× concentrated spray solution of the lipopeptide compound of Formula I(a) stock diluted in water with concentrations according to table 22, table 23, table 25 or table 26, respectively, was prepared. Each well contained 2% DMSO and 0.025% Tween20. Accordingly, a second set of master plates with 1× concentrated malonomicin stock diluted in water was prepared. Each well of the second set contained 0.025% Tween20. Leaf segments placed on agar in 24well plates were sprayed with 8 μl of solution from the master plate containing the lipopeptide compound of Formula I(a) and the leaf segments were let to dry, followed 2 hours later by 8 μl of solution from the master plate containing malonomicin. After second sprays had dried, leaf segments were infected with fungal spores to obtain a preventive application timing. Alternatively, leaf segments infected one day before spray of the compounds were used, resulting in a curative spray timing. In addition, several plates were produced where leaf segments were sprayed 2× in absence of test compound (with only DMSO and Tween20), representing the untreated check samples. For each leaf segment percent leaf coverage of disease symptoms was assessed. Percent leaf coverage reduction relative to the untreated check was calculated. Efficacy of the mixtures was tested in duplicate and on different fungal species. Reported efficacy values are the average of two replicate results.

Puccinia recondita (EPPO Code: PUCCRE) with Preventive Spray Timing.

Wheat (cultivar Kanzler) leaf segments are placed on agar in multiwell plates (24-well format) and sprayed with test solutions (8 ul per well). After drying, the leaf disks are inoculated with a spore suspension of the fungus. After appropriate incubation the activity of a compound is assessed 8 dpi (days after inoculation) as preventive fungicidal activity.

Puccinia recondita (EPPO Code: PUCCRE) with Curative Spray Timing.

Wheat (cultivar Kanzler) leaf segments are placed on agar in multiwell plates (24-well format). The leaf disks are then inoculated with a spore suspension of the fungus. One day after inoculation the test solution is sprayed (8 ul per well). After appropriate incubation the activity of a compound is assessed 8 dpi (days after inoculation) as curative fungicidal activity

Blumeria graminis f.sp. tritici (EPPO code: ERYSGT) with preventive spray timing

Wheat (cultivar Kanzler) leaf segments are placed on agar in multiwell plates (24-well format) and sprayed with test solutions (8 ul per well). After drying, the leaf disks are inoculated with spores of the fungus. After appropriate incubation the activity of a compound is assessed 7 dpi (days post inoculation) as preventive fungicidal activity.

Parastagonospora nodorum (EPPO Code: LEPTNO)

Wheat (cultivar Kanzler) leaf segments are placed on agar in multiwell plates (24-well format) and sprayed with test solutions (8 ul per well). After drying, the leaf disks are inoculated with a spore suspension of the fungus. After appropriate incubation the activity of a compound is assessed 4 dpi (days after inoculation) as preventive fungicidal activity.

### Results

TABLE 28
Control of Puccinia recondita (preventive) for solo compounds
and mixtures. The plate design including the concentration of compound
of Formula I(a) and malonomicin are indicated in Table 22 and 23.
Values indicate control of fungal growth (% reduction of symtoms
on the leaf segment as compared to untreated check).

| | (1) | (2) | (3) | (4) | (5) | (6) |
|---|---|---|---|---|---|---|
| 1-A | 80 | 100 | 100 | 100 | 95 | 80 |
| 1-B | 70 | 100 | 100 | 95 | 95 | 70 |
| 1-C | 70 | 100 | 100 | 90 | 80 | 60 |
| 1-D | 80 | 100 | 95 | 90 | 50 | 35 |
| 2-A | 10 | 100 | 100 | 80 | 0 | 0 |
| 2-B | 0 | 100 | 100 | 80 | 25 | 10 |
| 2-C | 0 | 100 | 100 | 90 | 45 | 10 |
| 2-D | 10 | 100 | 100 | 80 | 10 | 0 |

For each of the assay conditions in Table 28 with efficacy of 50% or more (effective mixture) the respective mixture ratio can be assigned from Table 24. Effective mixtures for the control of Puccinia recondita (preventive) were found from 64:1 to 1:270.

TABLE 29
Control of Puccinia recondita (curative) for solo compounds and
mixtures. The plate design including the concentration of compound
of Formula I(a) and malonomicin are indicated in Table 22 and Table
23. Values indicate control of fungal growth (% reduction of symtoms
on the leaf segment as compared to untreated check).

| | (1) | (2) | (3) | (4) | (5) | (6) |
|---|---|---|---|---|---|---|
| 1-A | 10 | 100 | 100 | 100 | 80 | 50 |
| 1-B | 0 | 100 | 100 | 100 | 95 | 90 |
| 1-C | 0 | 100 | 100 | 100 | 90 | 60 |
| 1-D | 0 | 100 | 100 | 100 | 100 | 80 |
| 2-A | 0 | 100 | 100 | 100 | 95 | 50 |
| 2-B | 0 | 100 | 100 | 100 | 100 | 80 |
| 2-C | 0 | 100 | 100 | 100 | 95 | 70 |
| 2-D | 10 | 100 | 100 | 100 | 70 | 35 |

For each of the assay conditions in Table 29 with efficacy of 50% or more (effective mixture) the respective mixture ratio can be assigned from Table 24. Effective mixtures for the control of Puccinia recondita (curative) were found from 64:1 to 1:270.

TABLE 30
Comparison of measured values for disease control of Puccinia recondita
(curative) (as reported in Table 29) with calculated values using
the formula from Colby for the same mixture. Numbers reported in the
table represent the difference from measured efficacy (in %) minus
calculated efficacy (in %). Values around 0 (zero) indicate additive
activity, while positive values suggest synergistic activity.

| | | | | | |
|---|---|---|---|---|---|
| (1) | (2) | (3) | (4) | (5) | (6) |

| | | | | | |
|---|---|---|---|---|---|
| 1-A | 0 | 0 | 0 | 7 | 9 |
| 1-B | 0 | 0 | 0 | 25 | 55 |
| 1-C | 0 | 0 | 0 | 20 | 25 |
| 1-D | 0 | 0 | 0 | 30 | 45 |
| 2-A | 0 | 0 | 0 | 25 | 15 |
| 2-B | 0 | 0 | 0 | 30 | 45 |
| 2-C | 0 | 0 | 0 | 25 | 35 |
| 2-D | | | | | |

TABLE 31
Control of Blumeria graminis f. sp. tritici (preventive)
for solo compounds and mixtures. The plate design including
the concentration of compound of Formula I(a) and malonomicin
are indicated in Table 25 and Table 27. Values indicate
control of fungal growth (% reduction of symtoms on the
leaf segment as compared to untreated check).

| | | | | | |
|---|---|---|---|---|---|
| (1) | (2) | (3) | (4) | (5) | (6) |

3-A

### Conclusions

Compound of Formula I(a) and malonomicin can be mixed to obtain full or partial control of various fungal pathogens. The mixture ratio of the two compounds can vary greatly in mixtures and still produce 50% or more control of the fungal growth when sprayed on a leaf. Examples are shown for ratio that range from 192:1 to 1:270, for several fungal species, including many examples with a ratio inbetween the mentioned ratios (ratio of compounds malonomicin:lipopeptide compound of Formula I(a). Surprisingly, the efficacy of many mixtures is better than the efficacy predicted based on the calculation from Colby, indicating that the mixture of compound of Formula I(a) with malonomicin has a synergistic effect on the control of fungal pathogens when sprayed on a leaf. This surprising synergistic effect was observed in tests to control Puccinia recondita.

1. A compound according to Formula (I) 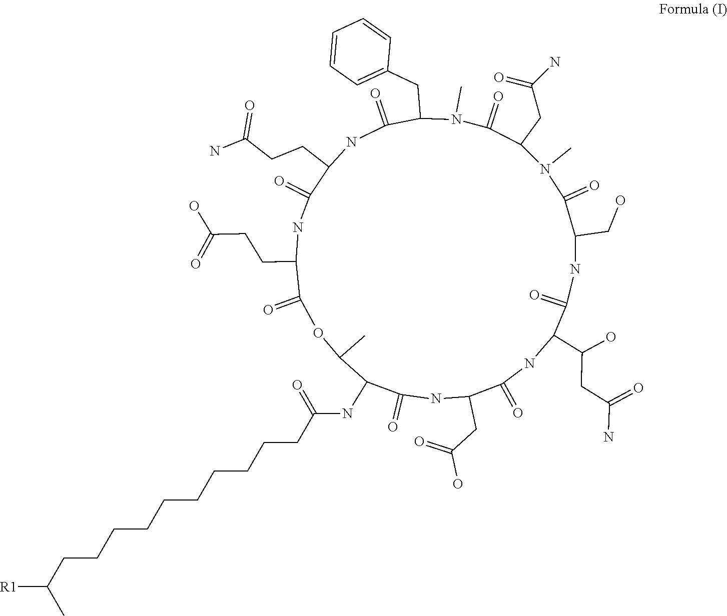
or a salt thereof, wherein R1 is CH₃ or C₂H₅.

2. A composition comprising the compound according to claim 1 and a microorganism that is able to produce the compound according to claim 1.

3. A composition comprising the compound according to claim 1 or the composition thereof, further comprising an auxiliary.

4. The compound according to claim 1 or the composition thereof, wherein the compound has fungicidal activity.

5. The composition according to claim 2, further comprising at least one additional ingredient having pesticidal activity and/or at least one plant growth regulator.

6. The composition according to claim 5, wherein the additional ingredient comprises cyclothiazomycin C, streptimidone and/or malonomicin, preferably malonomicin.

7. A process for producing the compound according to claim 1 or the composition thereof, comprising cultivating a microorganism in a suitable fermentation medium under conditions that allow production of the compound or the composition.

8. The composition according to claim 2, or the process thereof wherein the microorganism comprises at least one nucleotide sequence that encodes a protein that has at least 80% identity to at least one amino acid sequence(s) according to SEQ ID NO: 47 to 91, preferably SEQ ID NO: 67 or SEQ ID NO: 68, preferably, wherein the at least one nucleotide sequence has at least 80% identity to at least one of the nucleotide sequences according to SEQ ID NO: 2 to 46, preferably SEQ ID NO: 22 or SEQ ID NO: 23.

9. The composition according to claim 2, or the process thereof, wherein the microorganism is a Streptomyces sp., preferably a Streptomyces chrestomyceticus, preferably a Streptomyces which comprises a 16SRNA sequence which has at least 98% identity to SEQ ID NO: 1, preferably Streptomyces sp. Saigon413 deposited with the Westerdijk Institute under accession number CBS149411.

10. A microorganisms which is a Streptomyces sp. Saigon413 deposited with the Westerdijk Institute with accession number CBS149411.

11. A method for controlling or preventing infestation of a plant, plant propagation material and/or harvested food crops by a phytopathogenic microorganism, by treating the plant, plant propagation material and/or harvested food crops, wherein an effective amount of the compound according to claim 1 or the composition thereof is applied to the plant, to a part thereof or a locus thereof, the plant propagation material and/or harvested food crops.

12. The method according to claim 11, wherein the effective amount comprises 0.001 g to 5 kg of the compound of Formula (I) per hectare.

13. The method according according to claim 11, wherein the plant propagation material is seed and the effective amount comprises 0.0001 to 50 g of the compound of Formula (I) per kg of seed.

14. The method according to claim 11, wherein the phytopathogenic microorganism is a fungus, preferably a fungus belonging to Blumeria, Botrytis sp, Cercospora sp. Fusarium sp. Glomerella, Microdochium Mycosphaerella, Zymoseptoria sp., Parastagonospora sp., Puccinia sp., Phaeosphaeria sp. Pyrenophora, Pyricularia sp, Sclerotinia sp. Zymoseptoria preferably the fungi belong to Blumeria graminis f.sp. tritici, Botrytis cinerea, Cercospora arachidicola, Fusarium culmorum, Glomerella lagenarium, Microdochium nivale Mycosphaerella arachidis, Parastagonospora nodorum, Puccinia recondite, Puccinia recondita f. sp. tritici, Phaeosphaeria nodorum, Pyrenophora teres, Pyricularia oryzae, Sclerotinia sclerotiorum, or Zymoseptoria tritici.

15. The method according to claim 11, wherein the plant comprises potato, tomato, grape, canola, cucurbits, groundnut, wheat, and/or barley, corn, rice, soya, banana.

16. Use of a compound according to claim 1, or a composition thereof as a pesticide, preferably as a fungicide, and/or as a priming agent.
